(19) United States
(12) Patent Application Publication
(10) Pub. No.: US 2016/0206715 A1
(43) Pub. Date: Jul. 21, 2016

(54) SPECIFIC MULTIVALENT VIRUS-LIKE PARTICLE VACCINES AND USES THEREOF

(71) Applicants: BULLET BIOTECHNOLOGY, INC., Menlo Park, CA (US); THE BOARD OF TRUSTEES OF THE LELAND STANDFORD JUNIOR UNIVERSITY, Palo Alto, CA (US)

(72) Inventors: Thomas THERIAULT, Menlo Park, CA (US); James Robert SWARTZ, menlo Park, CA (US)

(21) Appl. No.: 14/777,383

(22) PCT Filed: Mar. 17, 2014
(86) PCT No.: PCT/US14/30788

§ 371 Date: Sep. 15, 2015

Related U.S. Application Data
(60) Provisional application No. 61/801,949, filed on Mar. 15, 2013.

(51) Int. Cl. A61K 39/00; C12N 7/00

(52) U.S. Cl. CPC A61K 39/0011; C12N 7/00; C12N 2730/10123; C12N 2730/10134

(57) ABSTRACT

The invention provides a VLP free of a viral genome comprising two or more display polypeptides, nucleic acid molecules, polymers of the nucleic acid, lipopolysaccharides, lipopeptides, peptidoglycans and/or small molecules.

Throughout this application various publications are referenced. The disclosures of these publications in their entirety are hereby incorporated by reference into this application in order to more fully describe the state of the art to which this invention pertains.

### BACKGROUND OF THE INVENTION

Autoimmune disease, cancer, and infectious disease are all major health problems without good solutions. The NIH estimates that 23.5 million Americans suffer from the more than 80 autoimmune diseases that have been described to date. A recent publication on 29 of the major autoimmune diseases estimates an even higher global prevalence of 7.6-9.4%. The American Cancer Society estimates that in 2012 more than 1,638,910 people were newly diagnosed with cancer and 577,190 people died from cancer. Many cancers, such as chronic lymphocytic leukemia (CLL) and non-Hodgkin lymphoma (NHL), are still fatal diseases with no cure. Patients can face years of treatments that are difficult to tolerate and have many adverse events. Five-year relative survival rates for NHL patients range from 85% for Follicular Lymphoma to 54% for Mantle-cell Lymphoma. Infectious disease also continues to be a problem: just to cite two examples, in 2010 approximately 899,000 Americans were living with HIV and on average there are 36,000 influenza-associated deaths every year.

Vaccines have utility in infectious diseases like measles and even flu, so a more effective vaccine for infectious diseases would clearly be valuable. Therapeutic vaccines also have very good potential in cancer: sipuleucel-T is now an approved dendritic-cell vaccine for prostate cancer and historical Idiotype (Id) vaccine programs demonstrated that a specific anti-Id immune response correlates strongly with progression-free and overall survival. (Ai 2009, Bendandi 2009, Bendandi 1999, Hsu 1997, Inoges 2011, Inoges 2011, Inoges 2009, Inoges 2006, Kwak 1992, Kwak 1996, Levy 2008, McCormick 2008, Schuster 2009) Unfortunately, previous vaccines did not consistently produce a strong immune response, and treatment with sipuleucel-T is a cumbersome process that is not effective in many patients. Antigen-specific approaches have been tried in autoimmune conditions as well, but with limited success.

The invention solves the problem of the art by providing novel specific combinations of display polypeptides, including immunostimulants, pathogen-associated molecular pattern receptor agonists, tumor-specific antigens, tumor-associated antigens and chemically synthesized compounds on multivalent VLPs of the invention that will induce an immune response sufficient to act as a therapeutic agent against cancer, infectious disease and autoimmune disease. (Basith 2011, Cooper 2009, Fontoura 2005. Hainsworth 2005, Hennessy 2010, Krieg 2006, Krieg 2008, Levy 2008, Lim 2010, Lim 2011, Miller 1982, Mizel 2010, Murata 2008, Siano 2008, Spina 2005, Witzig 2005, Zimmerman 2012, Zimmermann 2008).

### SUMMARY OF THE INVENTION

The multivalent virus-like particle (VLP) of the invention mimics the polyvalent nature of known pathogens, so that the invention may generate a stronger immune response than previously available known conjugates. In an embodiment, the compositions of the invention may stimulate an immune response towards a Th1, Th2, or Th1/Th2 type response to maximize the anti-tumor effect. The invention provides a personalized therapeutic vaccine that overcomes existing immune tolerance of the cancer while maintaining good tolerability, for improved survival and quality of life for patients.

In one embodiment, the multivalent VLPs of the invention are fundamentally different from other approaches in that they incorporate multiple particular, immune stimulants and copies of Id onto each VLP. The multivalent VLPs are designed to have stronger, more consistent immune stimulation and can be manufactured in a short period of time, e.g., one month, enabling its use, e.g., prior to, with, or following chemotherapy.

### BRIEF DESCRIPTION OF THE FIGURES

FIG. 1. Amino acid and nucleotide sequences of a Hepatitis B core antigen (HBC). M indicates the site of incorporation of the nnAA in the Hep B core.

FIG. 2. Amino acid and nucleotide sequences of a flagellin molecule.

FIG. 3. Amino acid and nucleotide sequences of a human GM-CSF.

FIG. 4. Amino acid and nucleotide sequences of a human IL-15.

FIG. 5. Nucleotide sequences of particular embodiments of CpG-X.

Sixteen different nucleotide sequences of embodiments of CpG that can be attached to VLPs are shown.

FIG. 6. Amino acid and nucleotide sequences of particular embodiments of Id antigens.

Panels a to t represent heavy and light chain variable region sequences from CLL patients.

FIG. 7. Amino acid and nucleotide sequences of eight embodiments of display polypeptides.

FIG. 8. Purification of the Hepatitis B Core.

Left Panel: Differential precipitation is observed between the HBC and other proteins in the CFPS reaction. Lane (1) marker; (2) soluble fraction of CFPS reaction; lanes (3) through (6) re-suspended precipitant from different concentrations of ammonium sulfate. Right Panel: Size exclusion chromatography following precipitant from lane 4 resuspended in buffer (lanes 7 and 8). Shown are the marker lane (1′) and representative fractions that were pooled. Yield was 4 mg of protein from a 10 ml CFPS reaction.

FIG. 9. Test of expression with a non-natural amino acid (nnAA) for muGM-CSF and Flagellin.

Varying buffer conditions tested in 3 hour (left) and overnight (right) reactions. Reaction 2 conditions run overnight yielded 200 ug/ml FLAG epitope-tagged, nnAA-containing muGM-CSF and over 650 ug/ml FLAG epitope-tagged, nnAA-containing flagellin proteins.

FIG. 10. Anti-FLAG antibody Western Blot analysis of “Click” chemistry.

Lane 1/1′: Molecular weight marker. Lane 2: Conjugated huGM-CSF (major band at 32 kDa). Lane 3 Native huGM-CSF 15.5 kDa. Lane 4: Conjugated muGM-CSF (major band at 32 kDa). Lane 5: Native muGM-CSF 15 kDa. Lane 6: Conjugated ScFV Id (minor band at 54 kDa). Lane 7: Native ScFV Id 37 kDa. Lane 2′: Conjugated flagellin (major band at 69 kDa). Lane 3′ Native Flagellin 52.5 kDa.

FIG. 11. Kinetic analysis of murine IL-15 receptor/ligand interaction.

Three concentrations of muIL-15 were analyzed with muIL-15 receptor-coated ForteBio sensors. Top trace: 200 nM, middle: 100 nM and bottom: 50 nM. The sensor data and the best fit (smooth curve) to a 1:1 (receptor:ligand) theoretical model are shown.

FIG. 12. HEK Blue hTLR-5 Assay.

HEK 293 cells expressing human TLR5 (InvivoGen hkb-htlr5) were assayed with varying concentrations of reference flagellin (AdipGen AG-40B-0025) for 6 (left bar) or 24 hours (right bar). This assay was used to verify free and VLP-attached flagellin activity.

FIG. 13. Analysis of azide activity.

Fluorescence and Coomassie-stained reducing SDS-PAGE gel images of azide-modified HBC and control proteins. 1. Size marker. 2. Purified HBC. 3. BSA (negative control). 4. BSA-Azide (positive control). 5. Precipitated HBC. Phosphine reaction with azide containing-proteins is confirmed by the fluorescence associated with the proteins in lanes 2, 4 and 5.

FIG. 14. Average body weights of mice during vaccination, initial tumor challenge (day 34, 0 post implantation (pi)) and tumor re-challenge (day 131, 97 pi).

FIG. 15. Average tumor volumes of 38C13 subcutaneous tumors.

FIG. 16. Survival (Time to endpoint, TTE) of mice challenged with 38C13 tumor cells. Kaplan-Meier curves are shown for 8 groups of animals with 38C13IgM-KLH and Blank VLP represented in both Panels. In Panel A, the curve for 38C13IgM-KLH has been nudged by −1% vertically to prevent overlap. In Panel B, the following nudges were used to prevent overlap: BB-005 (−1%), BB-004 (+1%), 38C13 IgM−KLH (−2%).

FIG. 17. Immune response results by event status for all groups.

Values obtained from the anti-Id immune response assay are plotted for all tumor challenge groups. Triangles indicate animals that reached the endpoint tumor burden. Circles indicate animals that remained tumor-free throughout the study.

### DETAILED DESCRIPTION OF THE INVENTION

### Definitions

“Vaccine” as used herein, is a preparation comprising a virus-like particle (VLP) or compositions of the invention that when administered stimulates an immune response and protective immunity in a mammal suffering from a disease, disorder or infection. A therapeutic vaccine may be administered during or after onset of a cancer, viral infection, or autoimmune disease. A prophylactic treatment vaccine may be administered prior to onset of a cancer, viral infection, or autoimmune disease and is intended to prevent onset of the cancer, viral infection or autoimmune disease.

The term “Id antigen” as used herein includes an idiotype protein (Id). The Id antigen may be an immunoglobulin (Ig), an Ig domain, or a fragment thereof. In another embodiment, the Id antigen may be a primary amino acid sequence for an Ig, an Ig fold, an Ig domain, or a fragment thereof. In another embodiment, the Id antigen may be a quaternary, tertiary, secondary, or primary structure for an Ig, Ig fold, Ig domain or a fragment thereof or a combination of a quaternary, tertiary, secondary, or primary structure for an Ig, Ig fold, Ig domain or a fragment thereof. The Id antigen may be expressed naturally as antibodies or immunoglobulins by B lymphocytes, as T-cell receptor (TCR) chains by T lymphocytes, as class I major histocompatibility complex (MHC) protein and beta-2 microglobulin (P2M) for antigen presentation, or class 11 MHC for antigen presentation.

For example, the Id antigen may be an antibody or immunoglobulin expressed by a B-cell malignancy or a T-cell receptor (TcR) expressed by a T-cell malignancy. The immunoglobulin may be a whole immunoglobulin or an immunoglobulin fragment. The fragment may include, but is not limited to, a Fab fragment, F(ab′) fragment, F(ab′) fragment or single chain variable fragment (scFv). In a preferred embodiment, the Id antigen is a scFv.

The T-cell receptor may comprise alpha- (α-) and beta- (β-) chains with Ig folds or domains in antigen-binding/MHC-binding Variable (V) region and disulfide bond-forming/interchain crosslinking Constant (C) region. The T-cell receptor may also comprise gamma- (γ-) and delta- (δ-) chains with Ig folds or domains in the V and C regions. The T-cell receptor may be a whole T-cell receptor or a T-cell receptor fragment. The fragment may be a single chain T-cell receptor. Immunoglobulin molecules consist of heavy (H) and light (L) chains, which comprise highly specific variable regions at their amino termini. The variable (V) regions of the H (V) and L (V_{L}) chains combine to form the unique antigen recognition or antigen combining site of the immunoglobulin (Ig) protein. The variable regions of an Ig molecule contain determinants (i.e., molecular shapes) that can be recognized as antigens or idiotypes.

The term “idiotype” refers to the unique set of antigenic or epitopic determinants (i.e., idiotopes) of an immunoglobulin, a B cell receptor or a T cell receptor.

The term “idiotope” refers to a single idiotypic epitope located along a portion of the V region of an immunoglobulin molecule.

The term “anti-idiotypic antibody” or grammatical equivalents refers to an antibody directed against an idiotype or one or more of the idiotopes on the V region of an Ig protein.

As used herein, the term “antibody” refers to intact antibody, or a portion or fragment or derivative thereof that competes with the intact antibody for specific binding and includes chimeric, humanized, fully human, and multispecific (e.g., bispecific) antibodies. The antibody may be a polyclonal antibody or monoclonal antibody, single chain Fv antibody fragments (scFv). Fab fragments, and F(ab)2 fragment.

As used herein “recombinant variable regions of immunoglobulin molecules” refers to variable regions of Ig molecules which are produced by molecular biological means. As shown herein, the variable domain of the heavy and light chains may be molecularly cloned from lymphoma cells and expressed in a host cell (e.g., by insertion into an expression vector followed by transfer of the expression vector into a host cell) or in a cell-free system; variable domains expressed in this manner are recombinant variable regions of immunoglobulin molecules. The recombinant variable regions of immunoglobulin molecules may be expressed as an immunoglobulin molecule comprising the recombinant variable regions operably linked to the appropriate constant region (i.e., C_{H} or C_{L}) (the constant region may comprise the constant region naturally associated with the recombinant variable region, as a Fab, F(ab′)₂ or Fab′ fragment comprising the variable domain of the heavy and light chains, the constant region of the light chain and a portion of the constant region of the heavy chain (the Fab, F(ab′), or Fab′ fragments may be created by digestion of a recombinant immunoglobulin molecule or alternatively, they may be produced by molecular biological means), or alternatively, as a single chain variable fragment fusion protein (scFv).

“Single-chain variable fragment” or “scFv” may be composed of an antibody light chain variable domain or region (“V_{L}”) and heavy chain variable region (“V_{H}”) connected by a short peptide linker. The peptide linker allows the structure to assume a conformation which is capable of binding to antigen (Bird 1988, Huston 1988).

A “recombinant variable region derived from a lymphoma cell” refers to a variable region which is molecularly cloned from RNA isolated from a lymphoma cell. The recombinant variable domain may be expressed as an entire immunoglobulin molecule or may be expressed as a fragment of an immunoglobulin molecule, including, for example, scFv molecules.

An “immune-enhancing cytokine” is a cytokine that is capable of enhancing the immune response when the cytokine is generated in situ or is administered to a subject. Immune-enhancing cytokine include, but are not limited to, granulocyte-macrophage colony stimulating factor (GM-CSF), interleukin-2 (IL-2), interleukin-3 (IL-3), interleukin-4 (IL-4), interleukin-12 (IL-12) and interleukin-15 (IL-15).

As used herein, a “subject” means a human or animal. Usually the animal is a vertebrate such as a primate, rodent, domestic animal or game animal. In certain embodiments of the aspects described herein, the subject is a mammal, e.g., a primate, e.g. a human. The terms, “patient” and “subject” are used interchangeably. A subject can be male or female.

Preferably, the subject is a mammal. The mammal can be a human, non-human primate, mouse, rat, dog, cat, horse, or cow, but are not limited to these examples. Mammals, other than humans, can be advantageously used as subjects that represent animal models of disorders associated with, e.g., cancer, autoimmune disease or inflammation. In addition, the methods and compositions described herein can be used to treat domesticated animals and/or pets.

An “adjuvant” is a compound which enhances or stimulates the immune response when administered with an antigen(s) or a vaccine of the invention.

The term “construct” as used herein refers to a recombinant nucleic acid molecule containing a desired coding sequence and appropriate nucleic acid sequences necessary for the expression of the operably linked coding sequence in a particular host organism. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals.

“Malignant cells isolated from a patient having a B-cell lymphoma” refers to the malignant or pathogenic B-cells found within the solid tumors characteristic of lymphoma (e.g., lymph nodes and spleen containing the tumor cells) or found within a blood sample in the case of leukemic B-cell lymphoma (e.g. CLL).

Administration to the subject can be by any appropriate route known in the art including, but not limited to, intramuscular injection, intravenous injection, subcutaneous injection, nasal spray and other mucosal delivery (e.g., transmucosal delivery), intradermal injection (e.g., with electroporation), electroincorporation, ultrasound, jet injector, and transdermal administration (e.g., topical patches). Exemplary modes of administration include, but are not limited to, injection, inhalation, or ingestion. Injection includes, without limitation, intravenous, intramuscular, intra-arterial, intrathecal, intraocular, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection or infusion. In some embodiments of the aspects described herein, administration is by intravenous infusion or injection.

According to the present invention, where administration includes a pharmaceutical formulation, preferably the formulation is a unit dosage containing a set dose or unit, set sub-dose or an appropriate fraction thereof, of the active ingredient (i.e., the VLP or compositions of the invention) administered over a set duration to elicit a sufficiently therapeutic immune response toward the antigen.

The multivalent VLP vaccines of the invention can be administered by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

When a VLP vaccine of the invention described herein is being given to a subject, a skilled artisan would understand that the dosage depends on several factor, including, but not limited to, the subject's weight, disease and progression thereof or tumor size or tumor progression. With respect to duration and frequency of treatment, it is typical for skilled clinicians to monitor subjects in order to determine whether the treatment is providing therapeutic benefit, and to determine whether to increase or decrease dosage, increase or decrease administration frequency, discontinue treatment, resume or make other alterations to the treatment regimen.

In human therapy, the multivalent VLP vaccines of the invention can be administered alone but may generally be administered in admixture with a suitable pharmaceutical excipient, diluent or carrier selected with regard to the intended route of administration and standard pharmaceutical practice.

In some embodiments of the present invention, the VLP or compositions of invention are administered parenterally, such administration can be, for example, intravenously, intra-arterially, intraperitoneally, intrathecally, intraventricularly, intrasternally, intracranially, intramuscularly, intraocularly or subcutaneously, or they may be administered by infusion techniques.

Additionally, the VLP or compositions of invention may be used in the form of a sterile aqueous solution which may contain other substances, for example, enough salts or glucose to make the solution isotonic with blood. The aqueous solutions may be suitably buffered, if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use.

Extemporaneous injection solutions and suspensions may be prepared from sterile powders, granules and tablets of the kind previously described.

In an embodiment, a non-limiting example of an administration protocol useful for the invention comprises multiple administrations of the multivalent VLP vaccine of the invention during an initial period (such as, for example, a six week period, with, for example, administration every two weeks).

By “effective amount” as used herein with respect to a multivalent VLP vaccine of the invention, is meant an amount of the multivalent VLP, administered to a subject that results in an immune response by the mammal so as to inhibit a cancer, viral infection or autoimmune disease. Further, an effective amount may include any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “inhibiting a tumor” may be measured in any way as is known and accepted in the art, including complete regression of the tumor(s) (complete response); reduction in size or volume of the tumor(s) or even a slowing in a previously observed growth of a tumor(s), e.g., at least a 30% decrease in the sum of the longest diameter (LD) of a tumor, taking as reference the baseline sum LD (partial response); mixed response (regression or stabilization of some tumors but not others)); or no apparent growth or progression of tumor(s) or neither sufficient shrinkage to qualify for partial response nor sufficient increase to qualify for progressive disease, taking as reference the smallest sum LD since the treatment started (stable disease).

Tumor or cancer status may also be assessed by sampling for the number, concentration or density of tumor or cancer cells, alone or with respect to a reference. Tumor or cancer status may also be assessed through the use of surrogate marker(s), such as ZAP-70 in chronic lymphocytic leukemia (Rassenti L Z, Huynh L, Toy T L, et al: ZAP-70 compared with immunoglobulin heavy-chain gene mutation status as a predictor of disease progression in chronic lymphocytic leukemia. N Engl J Med 2004 Aug. 26; 351(9):893-901; Crespo M, Bosch F, Villamor N, et al: ZAP-70 expression as a surrogate for immunoglobulin-variable-region mutations in chronic lymphocytic leukemia. N Engl J Med 2003 May 1; 348(18):1764-1765), followed over time to assess changes in tumor or cancer status. In the case of leukemias, bone marrow samples may be used to assess tumor or cancer status as well as complete blood count (CBC) for red blood cells, white blood cells, and platelets.

As used herein, “treating” means using a therapy to ameliorate a disease or disorder or one or more of the biological manifestations of the disease or disorder; to directly or indirectly interfere with (a) one or more points in the biological cascade that leads to, or is responsible for, the disease or disorder or (b) one or more of the biological manifestations of the disease or disorder; to alleviate one or more of the symptoms, effects or side effects associated with the disease or disorder or one or more of the symptoms or disorder or treatment thereof; or to slow the progression of the disease or disorder or one or more of the biological manifestations of the disease or disorder. Treatment includes eliciting a clinically significant response without excessive levels of side effects. Treatment may also include improving quality of life for a subject suffering from the disease or disorder (e.g., a subject suffering from a cancer may receive a lower dose of an anti-cancer drug that cause side-effects when the subject is immunized with a composition of the invention described herein). Throughout the specification, compositions of the invention and methods for the use thereof are provided and are chosen to provide suitable treatment for subjects in need thereof.

In some embodiments, treatment with a composition of the invention described herein induces and/or sustains an immune response in a subject. Immune responses include innate immune response, adaptive immune response, or both. Innate immune response may be mediated by neutrophils, macrophages, natural killer cells (NK cells), and/or dendritic cells. Adaptive immune response includes humoral responses (i.e., the production of antibodies), cellular responses (i.e., proliferation and stimulation of T-lymphocytes), or both. Measurement of activation and duration of cellular response are by any known methods including, for example, cytotoxic T-lymphocyte (CTL) assays. Humoral responses are also measured by known methods including isolation and quantitation of antibody titers specific to the compositions of the invention (e.g., vaccines) such as IgG or IgM antibody fractions.

In some embodiments, the methods of treatment (e.g., immunotherapy) described herein is used as a stand-alone therapy without combining with any other therapy.

In some embodiments, the methods of treatment (e.g., immunotherapy) described herein provide adjunct therapy to any other therapy, e.g., cancer therapy, prescribed for a subject. In additional embodiments, the methods of treatment (e.g., immunotherapy) described herein are administered in combination with radiotherapy, chemotherapy, gene therapy or surgery. The combination is such that the method of treatment (e.g., immunotherapy) described herein is administered prior to, with or following radiotherapy, chemotherapy, gene therapy or surgery.

Alternatively, the effect of anti-disease or disorder treatment (e.g., a cancer treatment) may be assessed by following the patient, e.g., by measuring and comparing survival time or time to disease progression (disease-free survival). Any assessment of response may be compared to individuals who did not receive the treatment or were treated with a placebo, or to individuals who received an alternative treatment.

As used herein, “preventing” is understood to refer to the prophylactic administration of a drug to substantially diminish the likelihood or severity of a condition or biological manifestation thereof, or to delay the onset of such condition or biological manifestation. One skilled in the art will appreciate that prevention is not an absolute term. Prophylactic therapy is appropriate, for example, when a subject is considered at high risk for developing a particular disease or disorder (e.g., cancer), such as when a subject has a strong family history of a disease or disorder or when a subject has been exposed to e.g., a disease causing agent, e.g., a carcinogen.

Other than in the operating examples, or where otherwise indicated, all numbers expressing quantities of ingredients or reaction conditions used herein should be understood as modified in all instances by the term “about”. The term “about” when used in connection with percentages can mean±1%.

The terms “a,” “an” and “the” include plural referents unless context clearly indicates otherwise. Similarly, the term “or” is intended to include “and” unless the context clearly indicates otherwise.

### COMPOSITIONS OF THE INVENTION

The invention provides for a VLP free of a viral genome comprising two or more display agents (e.g. polypeptides, nucleic acid molecules, polymers of a nucleic acid molecule, lipopolysaccharides, lipopeptides, peptidoglycans and/or small molecules). The VLP may be an isolated VLP or purified VLP. The display agents may be joined to the surface of the VLP. Additionally or alternatively, the agents may be contained within the VLP. In one embodiment, the VLP of the invention may be a stable icosahedral VLP. In accordance with the practice of the invention, the two or more display agents may be a whole agent (e.g. whole polypeptides, nucleic acid molecules, polymers of a nucleic acid molecule, lipopolysaccharides and/or small molecules) or a fragment or portion thereof.

The VLP free of a viral genome of the invention may comprise virus coat polypeptides derived from any of an Adenoviridae, Picornaviridae, Herpesviridae, Hepadnaviridae, Flaviviridae, Retroviridae, Orthomyxoviridae, Paramyxoviridae, Papillomaviridae, Rhabdoviridae, Togaviridae or Paroviridae families.

Specifically, examples of viruses from which the virus coat proteins may be derived include but are not limited to any of a bacteriophage, adenovirus, coxsackievirus, Hepatitis A virus, poliovirus, Rhinovirus, Herpes simplex virus, Varicella-zoster virus, Epstein-Barr virus, Human cytomegalovirus, Human herpes virus, Hepatitis B virus, Hepatitis C virus, yellow fever virus, dengue virus, West Nile virus. HIV, Influenza virus, Measles virus, Mumps virus, Parainfluenza virus, Respiratory syncytial virus, Human metapneumovirus, Human papillomavirus, Rabies virus, Rubella virus, Human bocavirus or Parvovirus, and Norovirus. In one embodiment, the bacteriophage may be a MS2 bacteriophage, P1 like viruses, P2 like viruses, T4 like viruses, P22 like viruses, and lambda-like viruses.

In accordance with the practice of the invention, a display polypeptide may be an antigen that includes any of a tumor associated antigen, a viral antigen and an Id antigen. Further, the tumor associated antigen, viral antigen and Id antigen may be a whole protein or a fragment thereof.

Examples of tumor-associated antigens include but are not limited to an Id antigen, 17-1 A, 707-AP, AFP, Annexin II, ART-4, BAGE, BAGE-1, b-catenin, BCG, bcr/abl, Bcr/abl el4a2 fusion junction, bcr-abl (polypeptide from translation of b3a2 transcript), bcr-abl (polypeptide from translation of b2a2 transcript), bcr-abl p210 (polypeptide from translation of b2a2 transcript), ber-ab1 p210 (polypeptide from translation of b3a2 transcript), bullous pemphigoid antigen-1, CA 19-9, CA125, CA215, CAG-3 cancer peptide, CAMEL tumor antigen, Cancer-testis antigen, Caspase-8, CCL3, CCL4, CDI6, CD20, CD3, CD30, CD55, CD63. CDC27, CDK-4, CDR3, CEA, cluster 5, cluster-5A, cyclin-dependent kinase-4. Cyp-B, DAM-1 0, DAM-6, Dek-cain, E7, EGFR, EGFRvII 1, EGP40, ELF2 M, EpCAM, FucGM 1, G250, GA733, GAGE, GAGE-1-8, gastrin cancer associated antigen, GD2, GD3, globoH, glycophorin, GM1, GM2, GM3, GnTV, Gn-T-V, gp100, Her-2/neu, HERV-K-ME, high molecular weight-associated antigen, high molecular weight proteoglycan (IMPG), HPV-16 E6, HPV-16 E7, HPVE6, HSP70-2M, HST-2, hTERT, human chorionic gonadotropin (HCG), Human milk fat globule (HMFG), iCE, KIAA0205, KK-LC-1, KM-HN-1, L6, LAGE-1, LcOse4Cer, LDLR/FUT, Lewis A, Lewis v/b, M protein, MAGE-1, MVC, MAGE-A1-12, MAGE-C2, MAGE-3, MART-1/Melan-A, MC1R, ME491, MUC1, MUC2, mucin, MUM-1, MUM-2, MUM-3, mutated p53, Myosin, MZ2-E. N9 neuraminidase, NA88, NA88-A, nasopharyngeal carcinoma antigen, NGA, NK1/c-3, Novel bcr/abl fusion BCR exons 1, 13, 14 with ABL exons 4, NY-ESO-1/LAGE-2, NY-ESO-1b, OC125, osteosarcoma associated antigen-1, P15, p190 mimor bcr-abl (ela2), p53, Pm1/RARa, Polysialic acid, PRAME tumor antigen, PSA, PSM, RU1, RU2, SAGE, SART-1, SART-2, SART-3, Sialyl LeA, Sp17, SSX-2, SSX-4, surface immunoglobulin, TAG-1, TAG-2, TEL/AML1, TP1, TRAG-3, TRP-1 (gp75), TRP-2, TRP2-INT2, hTRT, tumor associated glycoprotein-72 (TAG-72), tyrosinase, u-PA, WT1, and XAGE-1b, or an immunostimulatory fragment of any of the above.

The tumor associated antigen may be found on breast cancer cells. Merely by way of example, the tumor associated antigen may be a tumor associated antigen of a malignant lymphoma, glycosphingolipid GD2, or cell surface receptors such as ErbB2.

In a preferred embodiment of the invention, the tumor associated antigen is any of a Her2/neu antigen, a Muc1 antigen, a CEA antigen, a MAGE-3 antigen, a NY-ESO-1 antigen (also referred to herein as NY-ESO-1/LAGE-2), or a CA125 antigen or a portion thereof.

Examples of B-cell malignancies include but are not limited to non-Hodgkin lymphoma (NHL), Hodgkin lymphoma, Burkitt's lymphoma, acute lymphocytic leukemia, lymphoblastic lymphomas, chronic lymphocytic leukemia (CLL), mantle cell lymphoma (MCL), multiple myeloma (MM), small lymphocytic lymphoma (SLL), B-cell prolymphocytic leukemia, lymphoplasmocytic leukemia, splenic marginal zone lymphoma, marginal zone lymphoma (extra-nodal or nodal), plasma cell neoplasms (e.g., plasma cell myeloma, plasmacytoma, monoclonal immunoglobulin deposition diseases, heavy chain diseases), mixed cell type diffuse aggressive lymphomas of adults, large cell type diffuse aggressive lymphomas of adults, large cell immunoblastic diffuse aggressive lymphomas of adults, small non-cleaved cell diffuse aggressive lymphomas of adults, and follicular lymphoma (e.g., Grades I, II, III or IV). In a preferred embodiment, the Id antigen is expressed by a CLL tumor. In another preferred embodiment, the Id antigen is expressed by a NHL tumor.

Examples of T-cell malignancies include but are not limited to chronic lymphocytic leukemia (CLL)(now called T cell prolymphocytic leukemia), large granular lymphocyte leukemia (T gamma lymphoproliferative disease), mycosis fungoides/Sezary syndrome, diffuse aggressive lymphomas of adults, peripheral T-cell lymphomas (mixed cell type and large cell, immunoblastic), adult T-cell leukemia/lymphoma, angiocentric lymphomas (lymphomatoid granulomatosis polymorphic reticulosis), acute lymphocytic leukemia, peripheral T-cell lymphoma not otherwise specified (PTCL-NOS), anaplastic large cell lymphoma, angioimmunoblastic lymphoma, cutaneous T-cell lymphoma and lymphoblastic lymphoma.

The invention also provides embodiments wherein one of the two or more display agents of the VLP is a viral antigen. The viral antigen may be from any virus such as a Poliovirus; HIV; Hepatitis B; Hepatitis C; Hepatitis E; Rabies; Herpes simplex virus (HSV); Varicella-zoster virus (VZV); Epstein-Barr virus (EBV); Influenza; Smallpox; Myxoma; Rhinovirus; Coronavirus; Rubella virus; Adenovirus; Papillomavirus; or Human T-cell leukemia virus (HTLV).

The invention also provides embodiments wherein one of the two or more display agents of the VLP is a cytokine. Examples of cytokines include but are not limited to GM-CSF, interleukin-2, -7, -12, -15, and a growth factor. In one embodiment, the cytokine induces an immune response predominantly of the Th1 type and may be an IFN-γ, TNFα, IL-2 and/or IL-12. In another embodiment, the cytokine induces an immune response predominantly of the Th2 type and may be an IL-4, IL-5, IL-6 and/or IL-10. In a further embodiment, the cytokine induces an immune response of both the Th1/Th2 type.

The invention further provides embodiments wherein one of the two or more display agents of the VLP is a TLR agonist. Examples of a TLR agonist include but are not limited to TLR 2, 3, 4, 5, 7, 8, or 9 agonist.

Examples of a TLR-4 agonist include but are not limited to bacterial lipopolysaccharide (LPS). VSV-G, and HMGB-1.

Examples of a TLR-5 agonist may include but are not limited to a flagellin, or portions or derivatives thereof.

Examples of a TLR7 agonist include but are not limited to imiquimod (3-(2-methylpropyl)-3,5,8-triazatricyclo[7.4.0.0^{2,6}]trideca-1(9),2(6),4,7,10,12-hexaen-7-amine or 1-(2-methylpropyl)-1H-imidazo[4,5-c]quinolin-4-amine), isatoribine, 852A, and thymidine homopolymer (ODN 17mer).

The invention further provides embodiments wherein one of the two or more display agents of the VLP is an immunostimulant. The immunostimulant may be a bacterial protein, an interferon or a cytokine or fragment thereof.

The invention further provides embodiments wherein one of the two or more display agents of the VLP is an immunostimulatory oligonucleotide. In one embodiment of the invention, the immunostimulatory oligonucleotide comprising an unmethylated cytosine is DNA, modified DNA, RNA, modified RNA, messenger RNA (mRNA) or peptide nucleic acid (PNA) or mixtures thereof. The DNA, modified DNA, RNA, modified RNA, messenger RNA (mRNA) or peptide nucleic acid (PNA) or mixtures thereof may comprise deoxyribose, ribose, morpholine, N-(2-aminoethyl)-glycine, phosphodiester bond, phosphorothioate bond, phosphorodiamidate bond, peptide bond or 5-octadiynyl deoxyuridine or mixtures thereof. In an embodiment, the DNA or modified DNA is an oligodeoxynucleotide or modified oligodeoxynucleotide. In another embodiment, the oligonucleotide or modified oligonucleotide is an oligonucleotide with phosphodiester bonds, phosphorothioate bonds or mixture thereof.

In an embodiment of the invention, the CpG comprises a sequence, 5′-TGACTGTGAACGTTCGAGATGA-3′. The nucleic acid molecule, oligonucleotide or CpG may be a modified oligonucleotide with a mixture of phosphodiester and phosphorothioate bonds in the sequence. T*G*A*C*T*G*T*G*A*ACGT*T*C*G*A*G*A*T*G*A or T*G*A*C*T*G*T*G*A*A*CG*T*T*C*G*A*G*A*T*G*A, or T*G*A*C*T*G*T*G*A*A*C*G*T*T*C*G*A*G*A*T*G*A, where * represents replacement of a phosphodiester bond with a phosphorothioate bond. Still other embodiments of the CpG incorporate an alkyne functional group into the molecule, for example, by coupling 5-octadiynyl dU {5-Oct-dU} to either the 5′ or 3′ end of the sequence, for example, {5-Oct-dU}-T*G*A*C*T*G*T*G*A*A*CG*T*T*C*G*A*G*A*T*G*A or T*G*A*C*T*G*T*G*A*A*CG*T*T*C*G*A*G*A*T*G*A-{5-Oct-dU}, respectively. The alkyne functional group may participate in a (3+2) cycloaddition click reaction with an azide functional group incorporated into a capsid protein of a VLP, resulting in VLP crosslinked to a CpG. A preferred CpG-X embodiment comprises T*G*A*C*T*G*T*G*A*A*CG*T*T*C*G*A*G*A*T*G*A-{5-Oct-dU}.

In an embodiment of the invention, the average amount of CpG attached to VLP may be an equivalent to 10 to 50 copies of CpG per VLP, 40 to 80 copies of CpG per VLP, 70 to 170 copies of CpG per VLP. In another embodiment, the CpG attached to VLP protein monomers may be in an amount such that the CpG to VLP weight ratio is equivalent to 1:1000 to 1:100, 1:100 to 1:10, 1:10 to 1:4, 1:4 to 1:2 or 1:2 to 1:1. In yet another embodiment, the CpG attached to VLP protein monomers is in an amount such that the CpG to VLP monomer ratios is equivalent to 1:24 to 1:12, 1:12 to 1:6, 1:6 to 1:3, 1:3 to 2:3 or 1:2 to 1:1.

For attachment of the display agents to the VLP, the virus coat polypeptides of the VLP may be modified to comprise at least one first unnatural amino acid (also referred to herein as non-natural amino acid or non-canonical amino acid (nnAA)) at a site of interest and the two or more display polypeptides may be modified to comprise at least one second unnatural amino acid, wherein the first unnatural amino acid is different from, and reactive with the second unnatural amino acid. An example of one first unnatural amino acid is azidohomoalanine. An example of a second unnatural amino acid is propargyloxyphenylalanine. The azide functional group of azidohomoalanine incorporated into a capsid protein of a VLP may participate in a (3+2) cycloaddition click reaction with an alkyne functional group of propargyloxyphenylalanine incorporated into a display agent, resulting in VLP crosslinked to a display agent. Other unnatural amino acid-containing capsid proteins within the same VLP may similarly participate in the (3+2) cycloaddition click reaction to produce a VLP with two or more display agents. In another embodiment, the VLP may display a polypeptide and a CpG. In another embodiment, the VLP may display a polypeptide and a nucleic acid or a modified nucleic acid. In another embodiment, the VLP may display two or more polypeptides and a CpG. In a separate embodiment, the VLP may display two or more polypeptides and a nucleic acid or a modified nucleic acid.

For example, the scFv may be fused to a bacterial immunity protein IM9. In another embodiment, the scFv fused to a bacterial immunity protein IM9 is displayed as a polypeptide on a VLP. In yet another embodiment, the fragment or reduced disulfide bonds of the F(ab′)2 fragment is attached or joined to a VLP through a bifunctional crosslinking agent.

In an embodiment of the invention, the VLP contains at least one or at least two unnatural amino acid per capsid monomer subunit. For example, at least one-twentieth of the total number of unnatural amino acids in a VLP may be used to attach a display polypeptide or nucleic acid. In another embodiment, about one fourth of the total number of unnatural amino acids in a VLP may be used to attach a display polypeptide or nucleic acid. In a further embodiment, about one-third of the total number of unnatural amino acids in a VLP may be used to attach a display polypeptide or nucleic acid. In yet another embodiment, about one half of the total number of unnatural amino acids in a VLP may be used to attach a display polypeptide or nucleic acid.

Also, in an embodiment of the invention, in the VLP, at least one-tenth of the viral coat proteins may display a polypeptide, nucleic acid molecule, polymer of a nucleic acid molecule, liposaccharide and/or a small molecule. In another embodiment, at least one-fifth of the viral coat proteins may display a polypeptide, nucleic acid molecule, polymer of a nucleic acid molecule, liposaccharide and/or a small molecule. In yet another embodiment, about half of the viral coat proteins may display a polypeptide, nucleic acid molecule, polymer of a nucleic acid molecule, liposaccharide and/or a small molecule. In a further embodiment, about two-thirds of the viral coat proteins may display a polypeptide, nucleic acid molecule, polymer of a nucleic acid molecule, liposaccharide and/or a small molecule. In yet another embodiment, nearly all of the viral coat proteins may display a polypeptide, nucleic acid molecule, polymer of a nucleic acid molecule, liposaccharide and/or a small molecule.

In yet another embodiment of the invention, the display polypeptides may include a tumor associated antigen, viral antigen or an Id antigen and one or more agents from the group of: GM-CSF, IL-15, Pam3SK4, poly (I:C), LPS, flagellin, imiquimod, and CpG-X to yield about 255 possible VLPs distinguishable on the basis of the presence or absence of a particular display polypeptides in a combination of display polypeptides along with either a tumor associated antigen, viral antigen or an Id antigen.

In another embodiment, the VLP free of a viral genome of the invention further comprises a 5-octadiynyl deoxyuridine or a modified deoxyuridine or a linker at the 3′ or 5′ end. In an embodiment, the linker at the 3′ or 5′ end comprises a chemical functionality selected from a set including but not limited to an alkyne, azide, carbonyl, amine or sulfhydryl group.

The two or more display agents may include but are not limited to any of a tumor associated antigen and an immunostimulatory oligonucleotide comprising an unmethylated cytosine; a tumor associated antigen and flagellin; a tumor associated antigen, flagellin and an immunostimulatory oligonucleotide comprising an unmethylated cytosine; a tumor associated antigen and interleukin 15 (IL-15); a tumor associated antigen, IL-15 and an immunostimulatory oligonucleotide comprising an unmethylated cytosine; a tumor associated antigen and granulocyte-macrophage colony-stimulating factor (GM-CSF); a tumor associated antigen, GM-CSF and an immunostimulatory oligonucleotide comprising an unmethylated cytosine; a tumor associated antigen, GM-CSF, flagellin, and an immunostimulatory oligonucleotide comprising an unmethylated cytosine; a tumor associated antigen and poly (I:C); a tumor associated antigen, poly (I:C) and an immunostimulatory oligonucleotide comprising an unmethylated cytosine; a tumor associated antigen and one or more Toll-like receptor (TLR) agonists; a tumor associated antigen and one or more immunostimulants; a tumor associated antigen, GM-CSF and IL-15; a tumor associated antigen and (S)-[2,3-Bis(palmitoyloxy)-(2-RS)-propyl]-N-palmitoyl-(R)-Cys-(S)-Ser-(S)-Lys4-OH lipohexapeptide (Pam3CSK4); a tumor associated antigen and a lipopolysaccharide (LPS); a tumor associated antigen and 3-(2-methylpropyl)-3,5,8-triazatricyclo[7.4.0.0^{2,6}]trideca-1(9),2(6),4,7,10,12-hexaen-7-amine (1-(2-methylpropyl)-1H-imidazo[4,5-c]quinolin-4-amine or imiquimod); a tumor associated antigen, poly (I:C) and imiquimod; a tumor associated antigen, CpG-X, Pam3CSK4, flagellin and an immunostimulatory oligonucleotide comprising an unmethylated cytosine; and a tumor associated antigen, CpG-X, Pam3CSK4, flagellin, GM-CSF and an immunostimulatory oligonucleotide comprising an unmethylated cytosine.

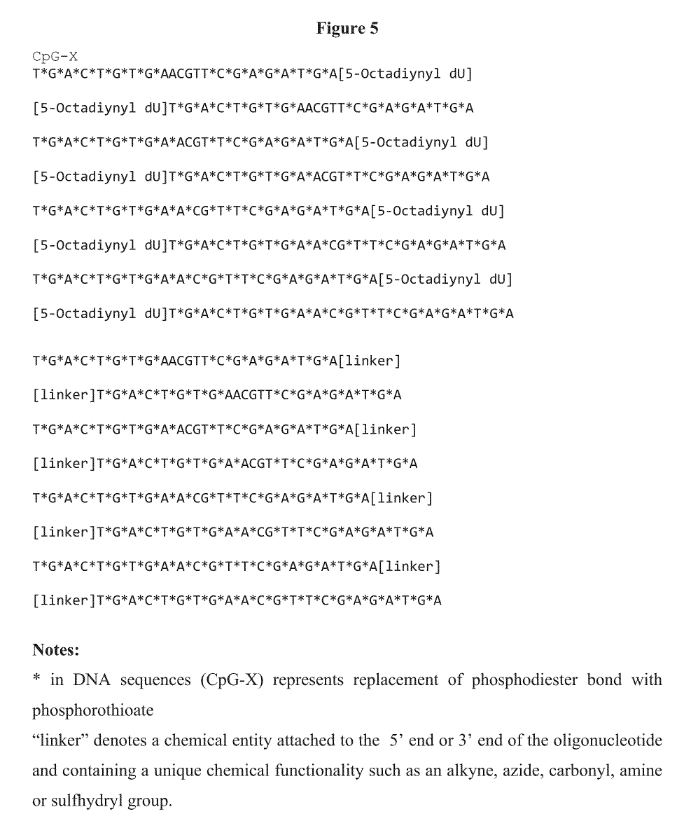

In an embodiment of the invention, the two or more display agents may include but are not limited to any of: a tumor associated antigen and an immunostimulatory oligonucleotide comprising an unmethylated CpG dinucleotide (CpG-X); a tumor associated antigen and flagellin; a tumor associated antigen, flagellin and CpG-X; a tumor associated antigen and interleukin 15 (IL-15); a tumor associated antigen, IL-15 and CpG-X; a tumor associated antigen and granulocyte-macrophage colony-stimulating factor (GM-CSF); a tumor associated antigen, GM-CSF and CpG-X; a tumor associated antigen, GM-CSF, CpG-X and flagellin; a tumor associated antigen and poly (I:C); a tumor associated antigen, poly (I:C) and CpG-X; a tumor associated antigen and a Toll-like receptor (TLR) agonist; and a tumor associated antigen and an immunostimulant. In one embodiment, a CpG-X has the nucleic acid sequence as shown in FIG. 5 or a portion thereof.

Examples of two or more display agents including a Her2/neu antigen include but are not limited to any of: a Her2/neu antigen or portion thereof and CpG-X; a Her2/neu antigen or portion thereof and flagellin; a Her2/neu antigen or portion thereof, flagellin and CpG-X; a Her2/neu antigen or portion thereof and IL-15; a Her2/neu antigen or portion thereof, IL-15 and CpG-X; a Her2/neu antigen or portion thereof and GM-CSF; a Her2/neu antigen or portion thereof, GM-CSF and CpG-X; a Her2/neu antigen, GM-CSF. CpG-X and flagellin; a Her2/neu antigen or portion thereof and poly (I:C); a Her2/neu antigen or portion thereof, poly (I:C) and CpG-X; a Her2/neu antigen or portion thereof and a TLR agonist; and a Her2/neu antigen or portion thereof and an immunostimulant.

Examples of two or more display agents including a Muc1 antigen include but are not limited to any of a Muc1 antigen and CpG-X; a Muc1 antigen and flagellin; a Muc1 antigen, flagellin and CpG-X; a Muc1 antigen and IL-15; a Muc1 antigen, IL-15 and CpG-X; a Muc1 antigen and GM-CSF; a Muc1 antigen, GM-CSF and CpG-X; a Muc1 antigen, GM-CSF, CpG-X and flagellin; a Muc1 antigen and poly (I:C); a Muc1 antigen, poly (I:C) and CpG-X; a Muc1 antigen and a TLR agonist; and a Muc1 antigen and an immunostimulant.

Examples of two or more display agents including a CEA antigen include but are not limited to a CEA antigen and CpG-X; a CEA antigen and flagellin; a CEA antigen, flagellin and CpG-X; a CEA antigen and IL-15; a CEA antigen, IL-15 and CpG-X; a CEA antigen and GM-CSF; a CEA antigen, GM-CSF and CpG-X; a CEA antigen, GM-CSF, CpG-X and flagellin; a CEA antigen and poly (I:C); a CEA antigen, poly (I:C) and CpG-X; a CEA antigen and a TLR agonist; and a CEA antigen and an immunostimulant.

Examples of two or more display agents including a MAGE-3 antigen include but are not limited to a MAGE-3 antigen and CpG-X; a MAGE-3 antigen and flagellin; a MAGE-3 antigen, flagellin and CpG-X; a MAGE-3 antigen and IL-15; a MAGE-3 antigen. IL-15 and CpG-X; a MAGE-3 antigen and GM-CSF; a MAGE-3 antigen, GM-CSF and CpG-X; a MAGE-3 antigen, GM-CSF, CpG-X and flagellin; a MAGE-3 antigen and poly (I:C); a MAGE-3 antigen, poly (I:C) and CpG-X; a MAGE-3 antigen and a TLR agonist; and a MAGE-3 antigen and an immunostimulant.

Examples of two or more display agents including a NY-ESO-1 antigen include but are not limited to a NY-ESO-1 antigen and CpG-X; a NY-ESO-1 antigen and flagellin; a NY-ESO-1 antigen, flagellin and CpG-X; a NY-ESO-1 antigen and IL-15; a NY-ESO-1 antigen, IL-15 and CpG-X; a NY-ESO-1 antigen and GM-CSF; a NY-ESO-1 antigen, GM-CSF and CpG-X; a NY-ESO-1 antigen, GM-CSF, CpG-X and flagellin; a NY-ESO-1 antigen and poly (I:C); a NY-ESO-1 antigen, poly (I:C) and CpG-X; a NY-ESO-1 antigen and a TLR agonist; and a NY-ESO-1 antigen and an immunostimulant.

Examples of two or more display agents including a CA125 antigen include but are not limited to any of a CA125 antigen and CpG-X; a CA125 antigen and flagellin; a CA125 antigen, flagellin and CpG-X; a CA125 antigen and IL-15; a CA125 antigen, IL-15 and CpG-X; a CA125 antigen and GM-CSF; a CA125 antigen, GM-CSF and CpG-X; a CA125 antigen, GM-CSF, CpG-X and flagellin; a CA125 antigen and poly (I:C); a CA125 antigen, poly (I:C) and CpG-X; a CA125 antigen and a TLR agonist; and a CA 125 antigen and an immunostimulant.

In another embodiment, the two or more display agents may include but are not limited to any of the combinations of Tumor associated antigen, flagellin and IL-15; Tumor associated antigen, flagellin, IL-15, and GM-CSF; Tumor associated antigen, flagellin, IL-15, GM-CSF, and poly (I:C); Tumor associated antigen, flagellin, IL-15, GM-CSF, poly (I:C), and TLR-agonist; Tumor associated antigen, flagellin, IL-15, GM-CSF, poly (I:C), TLR-agonist, and CpG-X; Tumor associated antigen, IL-15 and GM-CSF; Tumor associated antigen, IL-15, GM-CSF and poly (I:C); Tumor associated antigen, IL-15, GM-CSF, poly (I:C) and TLR-agonist; Tumor associated antigen, IL-15, GM-CSF, poly (I:C), TLR-agonist, and CpG-X; Tumor associated antigen, GM-CSF and poly (I:C); Tumor associated antigen. GM-CSF, poly (I:C) and TLR-agonist; Tumor associated antigen, GM-CSF, poly (I:C), TLR-agonist and CpG-X; Tumor associated antigen, poly (I:C) and TLR-agonist; Tumor associated antigen, poly (I:C), TLR-agonist and CpG-X; Tumor associated antigen, flagellin and GM-CSF; Tumor associated antigen, flagellin, GM-CSF and poly (I:C); Tumor associated antigen, flagellin, GM-CSF, poly (I:C) and TLR-agonist; Tumor associated antigen, flagellin, GM-CSF, poly (I:C), TLR-agonist and CpG-X; Tumor associated antigen, flagellin and poly (I:C); Tumor associated antigen, flagellin, poly (I:C) and TLR-agonist; Tumor associated antigen, flagellin, poly (I:C), TLR-agonist and CpG-X; Tumor associated antigen, flagellin and TLR-agonist; Tumor associated antigen, flagellin, TLR-agonist and CpG-X; Tumor associated antigen, flagellin, IL-15 and poly (I:C); Tumor associated antigen, flagellin, IL-15, poly (I:C) and TLR-agonist; Tumor associated antigen, flagellin, IL-15, poly (I:C), TLR-agonist and CpG-X; Tumor associated antigen, flagellin, IL-15 and GM-CSF; Tumor associated antigen, flagellin, IL-15, GM-CSF and TLR-agonist; Tumor associated antigen, flagellin, IL-15, GM-CSF, TLR-agonist and CpG-X; Tumor associated antigen, flagellin, IL-15. GM-CSF, poly (I:C) and CpG-X; Tumor associated antigen, GM-CSF and poly (I:C); Tumor associated antigen, IL-15, GM-CSF, poly (I:C), TLR-agonist; and Tumor associated antigen, IL-15, GM-CSF, poly (I:C), TLR-agonist, and CpG-X.

The invention also provides embodiments wherein one of the two or more display agents includes a first Id antigen. In one embodiment, the VLP further comprises a second Id antigen that is different from the first Id antigen. In another embodiment, the VLP further comprises a third Id antigen that is different from the first and second Id antigens.

Examples of the two or more display agents having an Id antigen include but are not limited to any of an Id antigen and a CpG-X; an Id antigen and flagellin; an Id antigen, flagellin and a CpG-X; an Id antigen and interleukin 15 (IL-15); an Id antigen. IL-15 and a CpG-X; an Id antigen and granulocyte-macrophage colony-stimulating factor (GM-CSF); an Id antigen, GM-CSF and a CpG-X; an Id antigen, GM-CSF, flagellin, and a CpG-X; an Id antigen and poly (I:C); an Id antigen, poly (I:C) and a CpG-X; an Id antigen and a Toll-like receptor (TLR) agonist; an Id antigen and an immunostimulant; an Id antigen, GM-CSF and IL-15; an Id antigen and (S)-[2,3-Bis(palmitoyloxy)-(2-RS)-propyl]-N-palmitoyl-(R)-Cys-(S)-Ser-(S)-Lys4-OH lipohexapeptide (Pam3CSK4); an Id antigen and a lipopolysaccharide (LPS); an Id antigen and 3-(2-methylpropyl)-3,5,8-triazatricyclo[7.4.0.0^{2,6}]trideca-1(9),2(6),4,7,10,12-hexaen-7-amine (1-(2-methylpropyl)-1H-imidazo[4,5-c]quinolin-4-amine or imiquimod); an Id antigen, poly (I:C) and imiquimod; an Id antigen. Pam3CSK4, flagellin and a CpG-X; and an Id antigen, Pam3CSK4, flagellin. GM-CSF and a CpG-X.

A preferred embodiment of the invention is a VLP free of a viral genome consisting of an Id antigen and a CpG-X. Another preferred embodiment is a VLP free of a viral genome consisting of an Id antigen and granulocyte-macrophage colony-stimulating factor (GM-CSF).

In an embodiment of the invention, the Id antigen is associated with an autoimmune disorder. Examples of autoimmune disorder include but are not limited to myasthenia gravis, primary biliary cirrhosis, dilated cardiomyopathy, myocarditis, autoimmune polyendocrine syndrome type 1 (APS-1), cystic fibrosis vasculitides, acquired hypoparathyroidism, Goodpasture syndrome, autoimmune hepatitis, Crohn disease, coronary artery disease, pemphigus foliaceus, pemphigus vulgaris, Guillain-Barre syndrome, type I diabetes, stiff man syndrome. Rasmussen encephalitis, autoimmune gastritis, Addison disease, type 1 diabetes, insulin hypoglycemic syndrome (Hirata disease), tacanthosis, systemic lupus erythematosus (SLE)), pernicious anemia, treatment-resistant Lyme arthritis, polyneuropathy, multiple sclerosis, demyelinating disease, rheumatic fever, atopic dermatitis, autoimmune hypothyroidism, vitilago, autoimmune thyroiditis, autoimmune Hashimoto thyroiditis, and celiac disease.

The autoimmune disorder may be a systemic autoimmune disorder. Examples of systemic autoimmune disorder include but are not limited to ACTH deficiency, myositis, dermatomyositis, polymyositis. SLE, Sjogren syndrome, systemic sclerosis, rheumatoid arthritis (RA), progressive systemic sclerosis), centromere-associated protein (systemic sclerosis, deimatomyositis, scleroderma, morphea, primary antiphospholipid syndrome, chronic idiopathic urticaria, connective tissue syndromes, necrotizing and cescentic glomerulonephritis (NCGN), system vasculitis, Wegener granulomatosis, Churg-Strauss syndrome, scleroderma. Raynaud syndrome, chronic liver disease, and systemic autoimmune disease.

The autoimmune disorder may be a plasma protein autoimmune disorder or cytokine autoimmune disorder. Examples of plasma protein autoimmune disorder or cytokine autoimmune disorder include but are not limited to an autoimmune CI deficiency, SLE membrane proliferative glomerulonephritis (MPGN), RA, systemic sclerosis, prolonged coagulation time, autoimmune thrombocytopenia purpura and atherosclerosis.

The Id antigen may be associated with a cancer or paraneoplastic autoimmune disorder. Examples of autoantigen associated with a cancer or paraneoplastic autoimmune disorder include but are not limited to neuropathy, small lung cell cancer, hepatocellular carcinoma, liver cancer, paraneoplastic pemphigus, paraneoplastic stiff man syndrome, paraneoplastic encephalomyelitis, sub-acute autonomic neuropathy, SLE, cancer-associated retinopathy, paraneoplastic opsoclonus myoclonus ataxia, lower motor neuron syndrome, and Lambert-Eaton myasthenic syndrome.

The invention also provides embodiments wherein one of the two or more display agents includes a viral antigen. In such embodiment, the two or more display agents may include any of: a viral antigen and CpG-X; a viral antigen and flagellin; a viral antigen, flagellin and CpG-X; a viral antigen and IL-15; a viral antigen, IL-15 and CpG-X; a viral antigen and GM-CSF; a viral antigen, GM-CSF and CpG-X; a viral antigen, GM-CSF, CpG-X and flagellin; a viral antigen and poly (I:C); a viral antigen, poly (I:C) and CpG-X; a viral antigen and a TLR agonist; and a viral antigen and an immunostimulant.

In a specific embodiment of the invention, the viral antigen is a HepB antigen. Examples of two or more display agents including a HepB antigen include but are not limited to any of a HepB antigen and CpG-X; a HepB antigen and flagellin; a HepB antigen, flagellin and CpG-X; a HepB antigen and IL-15; a HepB antigen, IL-15 and CpG-X; a HepB antigen and GM-CSF; a HepB antigen, GM-CSF and CpG-X; a HepB antigen, GM-CSF, CpG-X and flagellin; a HepB antigen and poly (I:C); a HepB antigen, poly (I:C) and CpG-X; a HepB antigen and a TLR agonist; and a HepB antigen and an immunostimulant. In accordance with the practice of the invention, these embodiments encompass portions of the agents above.

The invention also provides embodiments wherein one of the two or more display agents of the VLP is a Nod-like receptor agonist. In such an embodiment the two or more display agents include any of the following a Nod-like receptor agonist and CpG-X; a Nod-like receptor agonist and flagellin; a Nod-like receptor agonist, flagellin and CpG-X; a Nod-like receptor agonist and IL-15; a Nod-like receptor agonist, IL-15 and CpG-X; a Nod-like receptor agonist and GM-CSF; a Nod-like receptor agonist, GM-CSF and CpG-X; a Nod-like receptor agonist, GM-CSF, CpG-X and flagellin; a Nod-like receptor agonist and poly (I:C); a Nod-like receptor agonist, poly (I:C) and CpG-X; a Nod-like receptor agonist and a TLR agonist; and a Nod-like receptor agonist and an immunostimulant.

In one example of the invention, the VLP of the invention, in addition to the two or more display agents further comprises an adjuvant. In one embodiment, the adjuvant may be an adjuvant for eliciting a predominantly Th1-type response. Examples of adjuvant include but are not limited to one or a combination of monophosphoryl lipid A, preferably 3de-O-acylated monophosphoryl lipid A, together with an aluminum salt; CpG-X; saponin, such as Quil A, or derivatives thereof, including QS21 and QS7; Escin; Digitonin; or Gypsophila or Chenopodium quinoa saponins.

In additional examples, the adjuvant may be a GM-CSF, a mineral salt, alum, alum combined with monophosphoryl lipid A of Enterobacteria (MPL), saponins. QS-21, Quil-A, ISCOMATRIX™, MF59™, Montanide™ ISA 51, Montanide™ ISA 720, AS02, liposomes and liposomal formulations, AS01, synthesized or specifically prepared microparticles and microcarriers, chitosan particles, depot-forming agents, Pluronic block co-polymers, specifically modified or prepared peptides, muramyl dipeptide, aminoalkyl glucosaminide 4-phosphates, RC529, bacterial toxoids, toxin fragments, agonists of Toll-Like Receptors 2, 3, 4, 5, 7, 8, or 9; adenine derivatives; immunostimulatory DNA; immunostimulatory RNA; imidazoquinoline amines, imidazopyridine amines, 6,7-fused cycloalkylimidazopyridine amines, 1,2-bridged imidazoquinoline amines; imiquimod; resiquimod; agonist for DC surface molecule CD40; type I interferons; poly I:C; bacterial lipopolysaccharide (LPS); VSV-G; HMGB-1; flagellin or portions or derivatives thereof; CpG-X; proinflammatory stimuli released from necrotic cells; urate crystals; activated components of the complement cascade; activated components of immune complexes; complement receptor agonists; cytokines; cytokine receptor agonists; or oxoadenine or a combination thereof. Examples of imidazoquinoline include resiquimod and imiquimod.

Additional non-limiting examples of adjuvants useful in the present invention include aluminum hydroxide, aluminum phosphate, and Freund's complete adjuvant (FCA). Freund's incomplete adjuvant (FIA), calcium phosphate, liposomes. Virosomes™, ISCOMS®, microspheres (PLA, PLG), MF-59 emulsion, monophosphoryl Lipid A (MPL), muramyl-1-analyl-d-isoglutamine (PAMPs; E. coli heat labile enterotoxin (LT), flagellin, saponins, and small-molecule immune potentiators (SMIPs) In accordance with the invention, the VLP may contain, within it, a therapeutic agent of interest (supra.).

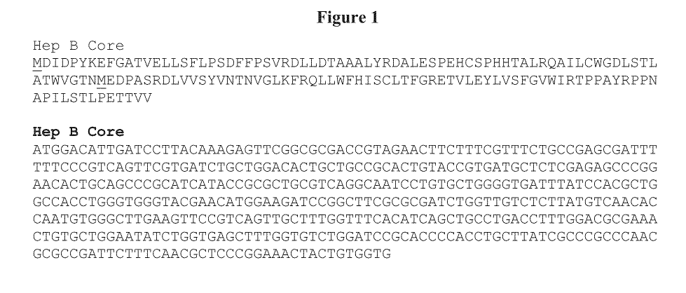

In a specific embodiment, the VLP may comprise a sequence of amino acid as set forth in FIG. 1.

In one embodiment of the invention, the HepB core sequence has the amino acid or nucleotide sequence as shown in FIG. 1 or a portion thereof.

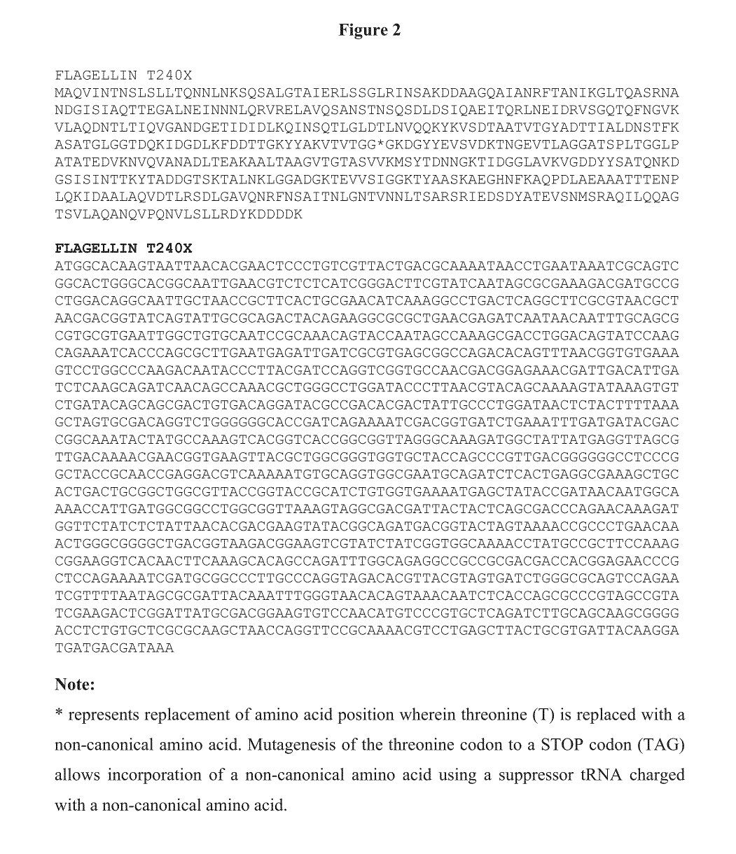

In an embodiment of the invention, the flagellin sequence has the amino acid or nucleotide sequence as shown in FIG. 2 or a portion thereof.

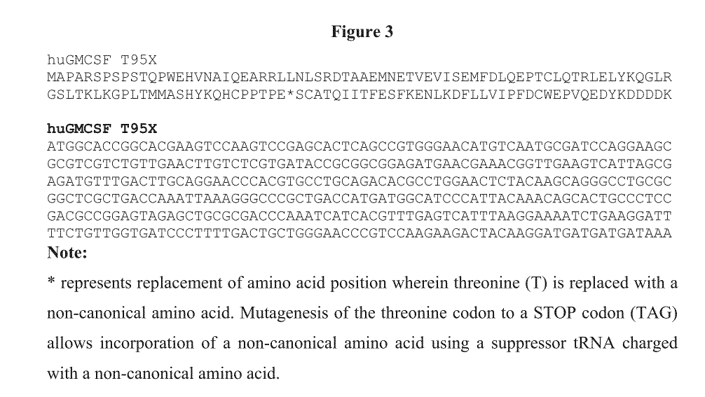

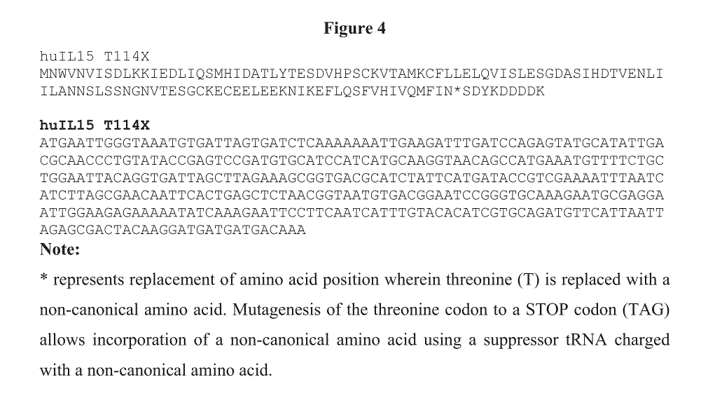

In one example, GM-CSF is human GM-CSF. In an embodiment of the invention, the human GM-CSF sequence may have an amino acid or nucleotide sequence as shown in FIG. 3 or a portion thereof.

In another example, interleukin (IL) is human interleukin. In an embodiment of the invention, the human IL is human IL-15 having an amino acid or nucleotide sequence as shown in FIG. 4 or a portion thereof.

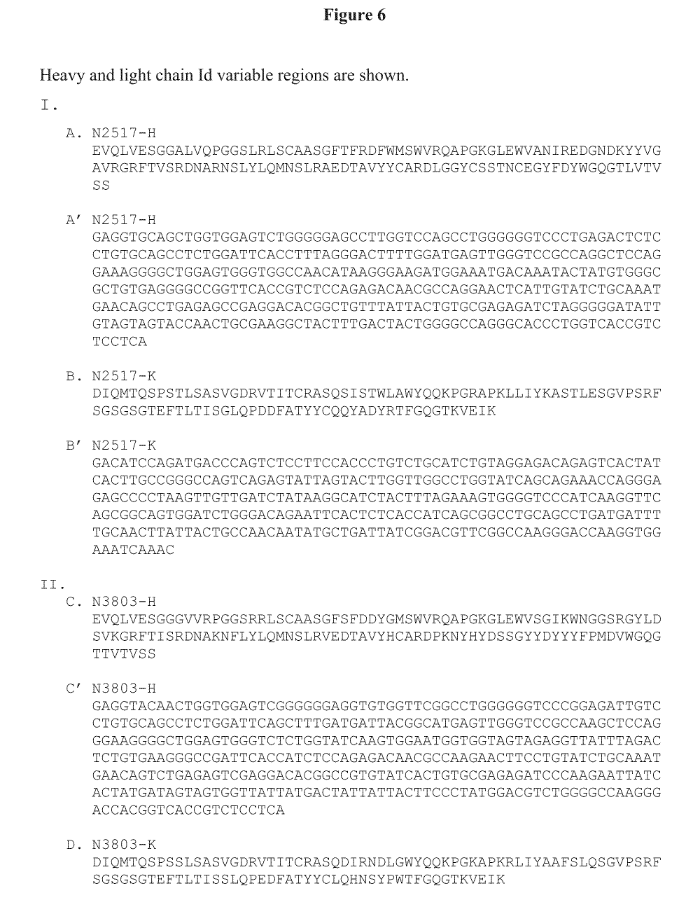

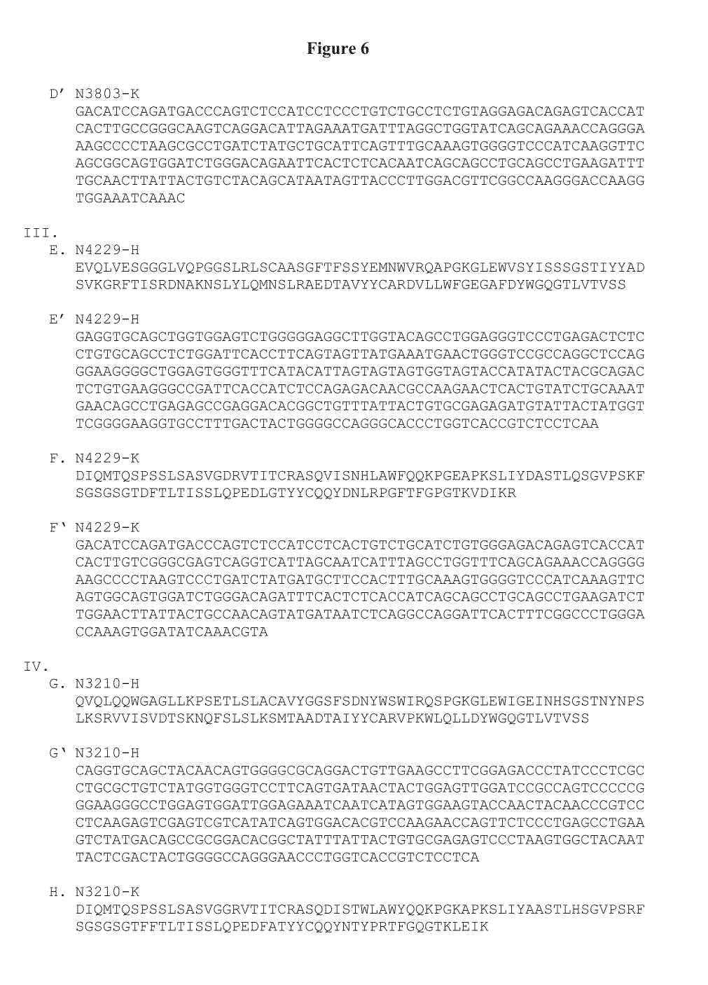

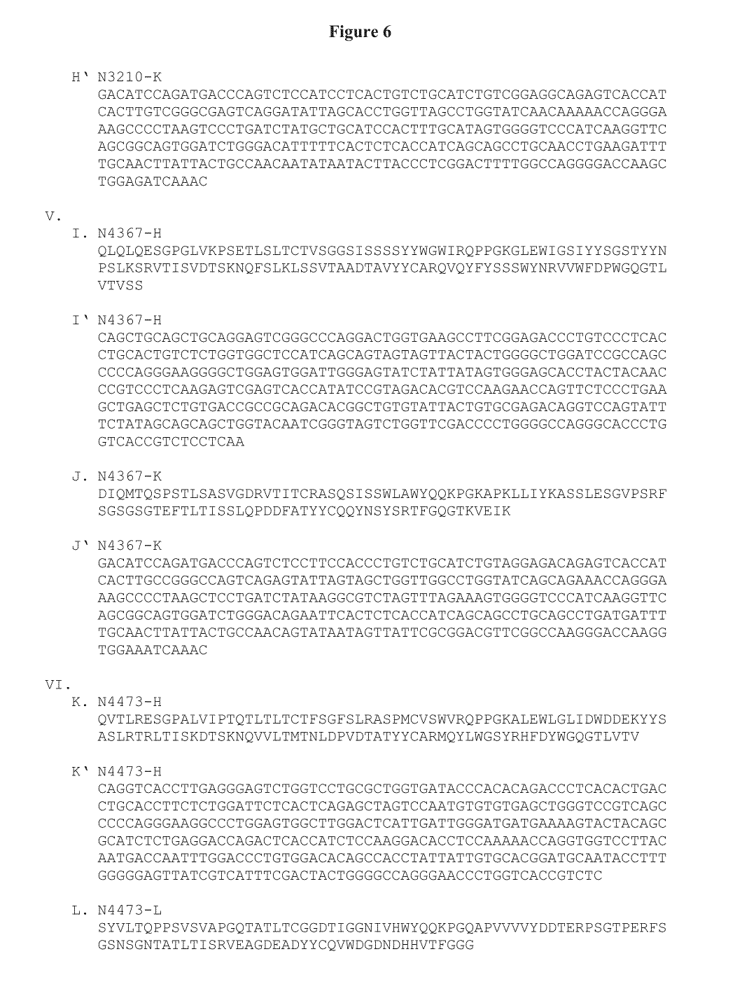

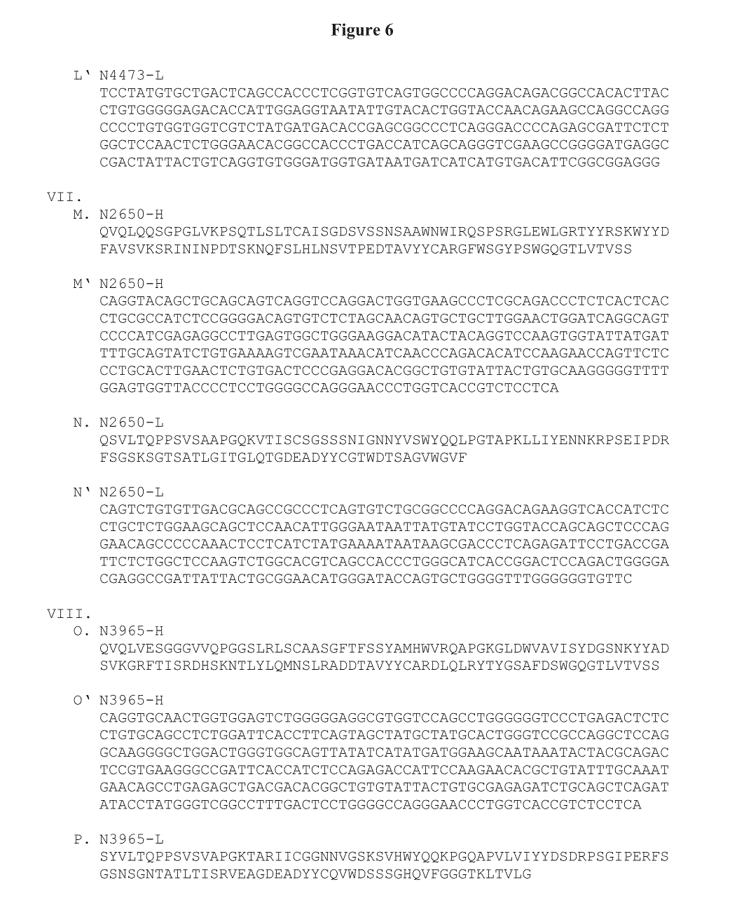

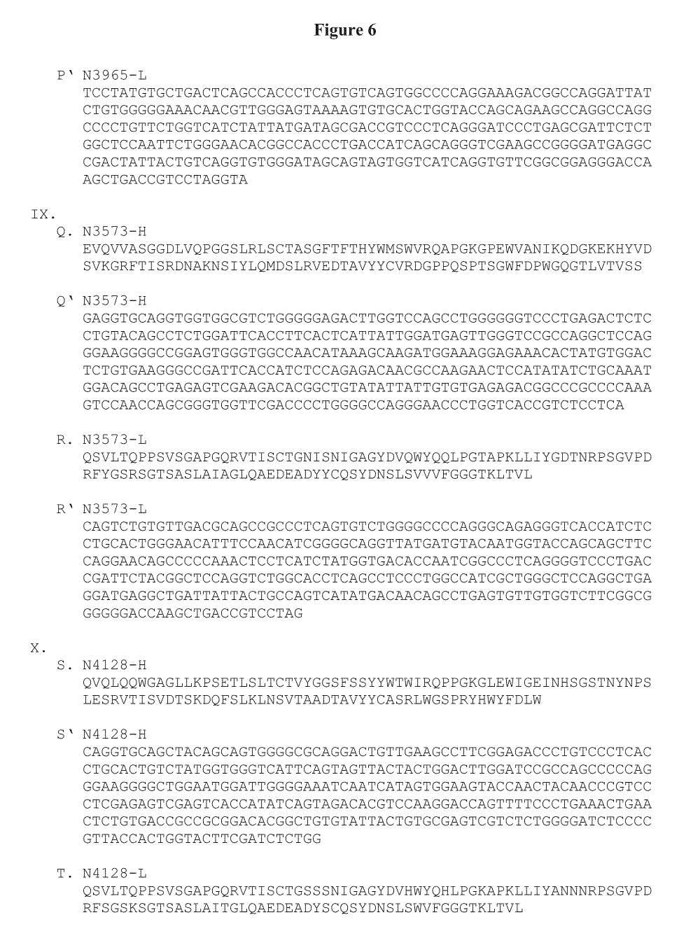

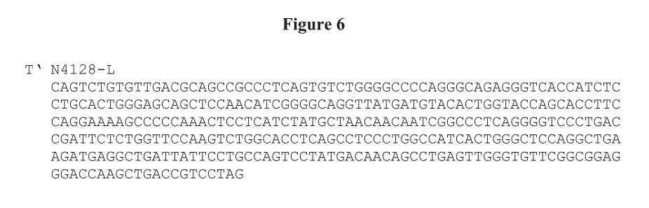

The Id antigen may be derived from a B cell receptor (BCR) or a T cell receptor (TCR). In an embodiment of the invention, the Id antigen may have an amino acid sequence or be encoded by a nucleotide sequence as shown in FIG. 6(I)(A) or (A′), respectively. In another embodiment, the Id antigen may have an amino acid sequence or be encoded by a nucleotide sequence as shown in FIG. 6 (I)(B) or (B′), respectively. In another embodiment, the Id antigen may have an amino acid sequence or be encoded by a nucleotide sequence as shown in FIG. 6 (II)(C) or (C′), respectively. In yet another embodiment, the Id antigen may have an amino acid sequence or be encoded by a nucleotide sequence as shown in FIG. 6 (II)(D) or (D′), respectively. In another embodiment, the Id antigen may have an amino acid sequence or be encoded by a nucleotide sequence as shown in FIG. 6 (III)(E) or (E′), respectively. In another embodiment, the Id antigen may have an amino acid sequence or be encoded by a nucleotide sequence as shown in FIG. 6 (III)(F) or (F′), respectively. In yet another embodiment, the Id antigen may have an amino acid sequence or be encoded by a nucleotide sequence as shown in FIG. 6 (IV)(G) or (G′), respectively. In another embodiment, the Id antigen may have an amino acid sequence or be encoded by a nucleotide sequence as shown in FIG. 6 (IV)(H) or (H′), respectively. In another embodiment, the Id antigen may have an amino acid sequence or be encoded by a nucleotide sequence as shown in FIG. 6 (V)(I) or (I′), respectively. In another embodiment, the Id antigen may have an amino acid sequence or be encoded by a nucleotide sequence as shown in FIG. 6 (V)(J) or (J′), respectively. In another embodiment, the Id antigen may have an amino acid sequence or be encoded by a nucleotide sequence as shown in FIG. 6 (VI)(K) or (K′), respectively. In another embodiment, the Id antigen may have an amino acid sequence or be encoded by a nucleotide sequence as shown in FIG. 6 (VI)(L) or (L′), respectively. In another embodiment, the Id antigen may have an amino acid sequence or be encoded by a nucleotide sequence as shown in FIG. 6 (VII)(M) or (M′), respectively. In yet another embodiment, the Id antigen may have an amino acid sequence or be encoded by a nucleotide sequence as shown in FIG. 6 (VII)(N) or (N′), respectively. In another embodiment, the Id antigen may have an amino acid sequence or be encoded by a nucleotide sequence as shown in FIG. 6 (VIII)(O) or (O′), respectively. In another embodiment, the Id antigen may have an amino acid sequence or be encoded by a nucleotide sequence as shown in FIG. 6 (VIII)(P) or (P′), respectively. In another embodiment, the Id antigen may have an amino acid sequence or be encoded by a nucleotide sequence as shown in FIG. 6 (IX)(Q) or (Q′), respectively. In another embodiment, the Id antigen may have an amino acid sequence or be encoded by a nucleotide sequence as shown in FIG. 6 (IX)(R) or (R′), respectively. In another embodiment, the Id antigen may have an amino acid sequence or be encoded by a nucleotide sequence as shown in FIG. 6 (X)(S) or (S′), respectively. In yet another embodiment, the Id antigen may have an amino acid sequence or be encoded by a nucleotide sequence as shown in FIG. 6 (X)(T) or (T′), respectively. In accordance with the practice of the invention, any of these embodiments may include a portion of any of the sequences above instead of the entirety.

In another embodiment, the Id antigen may have an amino acid sequence or be encoded by a nucleotide sequence as shown in FIG. 6 (I)(A) or (A′), respectively and FIG. 6 (I)(B) or (B′), respectively. In another embodiment, the Id antigen may have an amino acid sequence or be encoded by a nucleotide sequence as shown in FIG. 6 (II)(C) or (C′), respectively and FIG. 6 (II)(D) or (D′), respectively. In another embodiment, the Id antigen may have an amino acid sequence or be encoded by a nucleotide sequence as shown in FIG. 6 (III)(E) or (E′), respectively and FIG. 6 (III)(F) or (F′), respectively. In another embodiment, the Id antigen may have an amino acid sequence or be encoded by a nucleotide sequence as shown in FIG. 6 (IV)(G) or (G′), respectively and FIG. 6 (IV)(H) or (H′), respectively. In another embodiment, the Id antigen may have an amino acid sequence or be encoded by a nucleotide sequence as shown in FIG. 6 (V)(I) or (I′), respectively and FIG. 6 (V)(J) or (J′), respectively. In another embodiment, the Id antigen may have an amino acid sequence or be encoded by a nucleotide sequence as shown in FIG. 6 (VI)(K) or (K′), respectively or FIG. 6 (VI)(L) or (L′), respectively.

In another embodiment, the Id antigen may have an amino acid sequence or be encoded by a nucleotide sequence as shown in FIG. 6 (VII)(M) or (M′), respectively and FIG. 6 (VII)(N) or (N′), respectively. In another embodiment, the Id antigen may have an amino acid sequence or be encoded by a nucleotide sequence as shown in FIG. 6 (VIII)(O) or (O′), respectively and FIG. 6 (VIII)(P) or (P′), respectively. In another embodiment, the Id antigen may have an amino acid sequence or be encoded by a nucleotide sequence as shown in FIG. 6 (IX)(Q) or (Q′), respectively and FIG. 6 (IX)(R) or (R′), respectively. In yet another embodiment, the Id antigen may have an amino acid sequence or be encoded by a nucleotide sequence as shown in FIG. 6 (X)(S) or (S′), respectively and FIG. 6 (X)(T) or (T′), respectively. In accordance with the practice of the invention, any of these embodiments may include a portion of any of the sequences above instead of the entirety.

In another embodiment, the Id antigen may be a scFv derived from any of the amino acid sequence provided in FIG. 6 (A) to (T) or any of the pair of amino acid sequences provided in FIG. 6 Roman numeral (I) to (X).

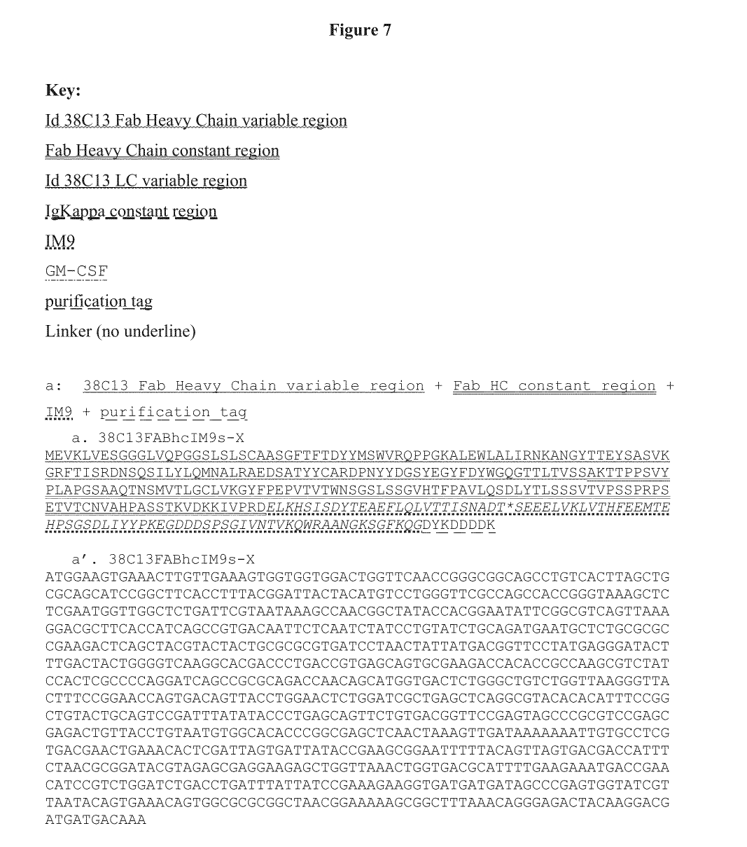

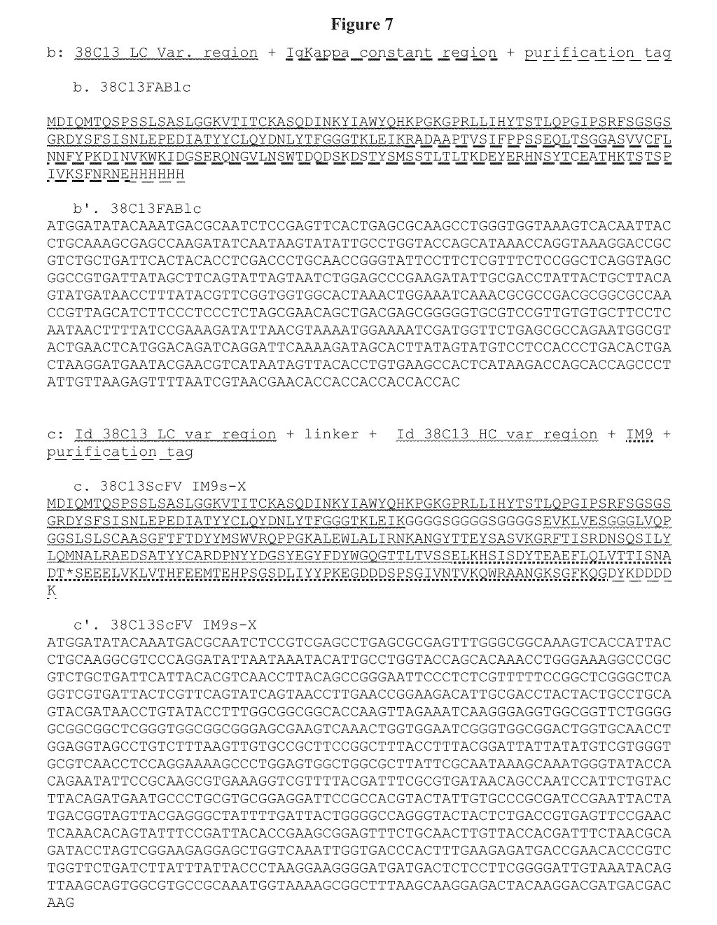

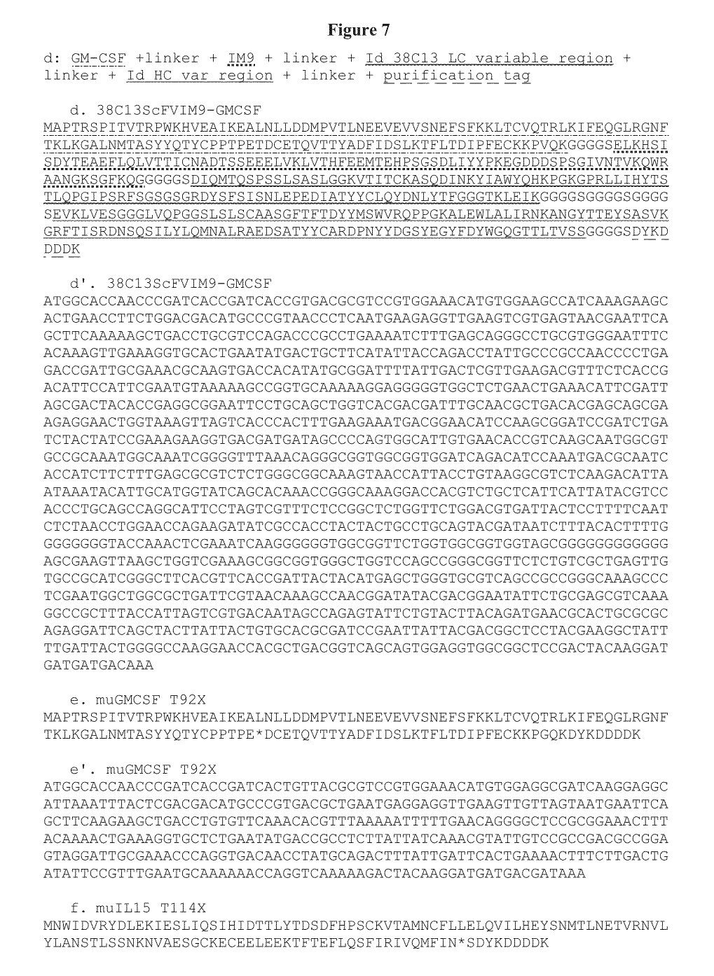

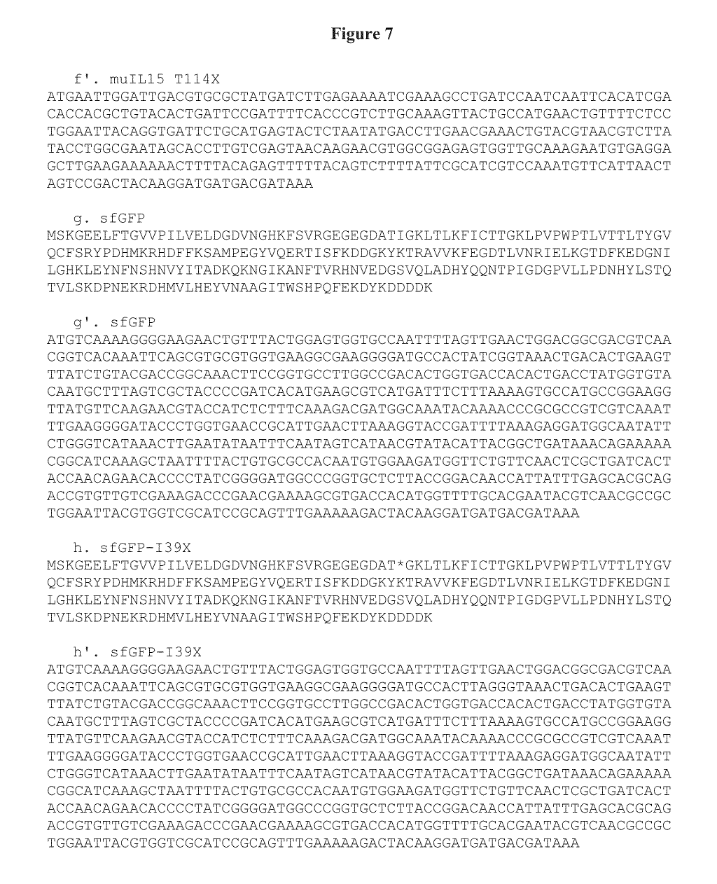

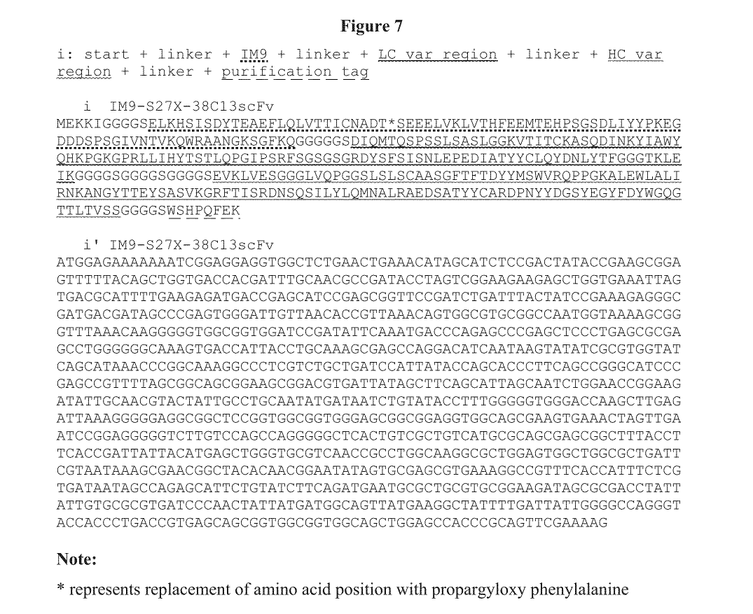

In another embodiment, the Id antigen may contain an amino acid sequence as shown in FIG. 7. Additionally, in an embodiment of the invention, the Id antigen comprises an immunoglobulin variable heavy (V_{H}) chain domain or sequence having an amino acid motif Q-(A or P)-(P or L)-G-(Q or K)-G-L-E-W-(M or V or I) immediately preceding a tripeptide motif, (G or A or S)-(X)-1, wherein X is any amino acid. The combined motifs are derived from 13 amino acids of framework 2 (FR2) for a subset of human immunoglobulin V_{H} chains, associated with certain human cancers, such as chronic lymphocytic leukemia (CLL). For example, the Id antigen may comprise any of the following sequences: QAPGQGLEWMG(X)I; QAPGQGLEWVG(X)I; QAPGQGLEWIG(X)I; QAPGKGLEWMG(X)I; QAPGKGLEWVG(X)I; QAPGKGLEWIG(X)I; QALGQGLEWMG(X)I; QALGQGLEWVG(X)I; QALGQGLEWIG(X)I; QALGKGLEWMG(X)I; QALGKGLEWVG(X)I; QALGKGLEWIG(X)I; QPPGQGLEWMG(X)I; QPPGQGLEWVG(X)I; QPPGQGLEWIG(X)I; QPPGKGLEWMG(X)I; QPPGKGLEWVG(X)I; QPPGKGLEWIG(X)I; QPLGQGLEWMG(X)I; QPLGQGLEWVG(X)I; QPLGQGLEWIG(X)I; QPLGKGLEWMG(X)I; QPLGKGLEWVG(X)I; QPLGKGLEWIG(X)I; QAPGQGLEWMA(X)I; QAPGQGLEWVA(X)I; QAPGQGLEWIA(X)I; QAPGKGLEWMA(X)I; QAPGKGLEWVA(X)I; QAPGKGLEWIA(X)I; QALGQGLEWMA(X)I; QALGQGLEWVA(X)I; QALGQGLEWIA(X)I; QALGKGLEWMA(X)I; QALGKGLEWVA(X)I; QALGKGLEWIA(X)I; QPPGQGLEWMA(X)I; QPPGQGLEWVA(X)I; QPPGQGLEWIA(X)I; QPPGKGLEWMA(X)I; QPPGKGLEWVA(X)I; QPPGKGLEWIA(X)I; QPLGQGLEWMA(X)I; QPLGQGLEWVA(X)I; QPLGQGLEWIA(X)I; QPLGKGLEWMA(X)I; QPLGKGLEWVA(X)I; QPLGKGLEWIA(X)I; QAPGQGLEWMS(X)I; QAPGQGLEWVS(X)I; QAPGQGLEWIS(X)I; QAPGKGLEWMS(X)I; QAPGKGLEWVS(X)I; QAPGKGLEWIS(X)I; QALGQGLEWMS(X)I; QALGQGLEWVS(X)I; QALGQGLEWIS(X)I; QALGKGLEWMS(X)I; QALGKGLEWVS(X)I; QALGKGLEWIS(X)I; QPPGQGLEWMS(X)I; QPPGQGLEWVS(X)I; QPPGQGLEWIS(X)I; QPPGKGLEWMS(X)I; QPPGKGLEWVS(X)I; QPPGKGLEWIS(X)I; QPLGQGLEWMS(X)I; QPLGQGLEWVS(X)I; QPLGQGLEWIS(X)I; QPLGKGLEWMS(X)I; QPLGKGLEWVS(X)I; or QPLGKGLEWIS(X)I; wherein X is any amino acid (e.g., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) or tyrosine (Y)).

In another embodiment of the invention the Id antigen comprises a variable heavy domain having an amino acid sequence of one of the following: YYMHWVRQAPGQGLEWMGRIN, YYMHWVRQAPGQGLEWMGWIN, YAISWVRQAPGQGLEWMGGII, YTISWVRQAPGQGLEWMGRII, YAISWVRQAPGQGLEWMGRII, YWMSWVRQAPGKGLEWVANIK, YAMSWVRQAPGKGLEWVSAIS, YAMSWVRQAPGKGLEWVSAIY, YAMSWVRQAPGKGLEWVSVIY, YAMHWVRQAPGKGLEWVAVIS, YYWSWIRQPPGKGLEWIGEIN, YYWCWIRQPLGKGLEWIGEIN, YYWSWIRQPPGKGLEWIGYIY, or YYWSWIRQPPGKGLEWIGEII. These sequences are derived from framework and complementary determining regions, CDRs, of human variable region genes.

In an embodiment of the invention, the average amount of Id antigen attached to VLP may be an equivalent to 10 to 50 copies of Id antigen per VLP, 40 to 80 copies of Id antigen per VLP, 70 to 170 copies of Id antigen per VLP, or 160 to 240 copies of Id antigen per VLP. In another embodiment, the Id antigen attached to VLP protein monomers may be in an amount such that the Id antigen to VLP weight ratio is equivalent to 1:1000 to 1:100, 1:100 to 1:10, 1:10 to 1:4, 1:4 to 1:2 or 1:2 to 1:1. In yet another embodiment, the Id antigen attached to VLP protein monomers is in an amount such that the Id antigen to VLP monomer ratios is equivalent to 1:24 to 1:12, 1:12 to 1:6, 1:6 to 1:3, 1:3 to 2:3 or 1:2 to 1:1.

In an embodiment of the invention, the average amount of GM-CSF attached to VLP may be an equivalent to 10 to 50 copies of GM-CSF per VLP, 40 to 80 copies of GM-CSF per VLP, 70 to 170 copies of GM-CSF per VLP, or 160 to 240 copies of GM-CSF per VLP. In another embodiment, the GM-CSF attached to VLP protein monomers may be in an amount such that the GM-CSF to VLP weight ratio is equivalent to 1:1000 to 1:100, 1:100 to 1:10, 1:10 to 1:4, 1:4 to 1:2 or 1:2 to 1:1. In yet another embodiment, the GM-CSF attached to VLP protein monomers is in an amount such that the GM-CSF to VLP monomer ratios is equivalent to 1:24 to 1:12, 1:12 to 1:6, 1:6 to 1:3, 1:3 to 2:3 or 1:2 to 1:1.

In one embodiment, the CpG and Id antigen may be attached to the VLP protein monomers in an amount such that the CpG to Id ratio is equivalent to 1:24 to 1:12, 1:12 to 1:6, 1:6 to 1:3, 1:3 to 2:3 or 1:2 to 1:1. In another embodiment, the GM-CSF and Id antigen are attached to the VLP protein monomers in an amount such that the GM-CSF to Id ratio is equivalent to 1:24 to 1:12, 1:12 to 1:6, 1:6 to 1:3, 1:3 to 2:3 or 1:2 to 1:1.

For example, a display polypeptide may comprise an amino acid sequence or be encoded by a nucleotide sequence as shown in any of FIG. 7a or a′, respectively, FIG. 7b or b′, respectively, FIG. 7c or c′, respectively, FIG. 7d or d′, respectively, FIG. 7e or e′, respectively, FIG. 7f, or f′, respectively FIG. 7g or g′, respectively, or FIG. 7h or h′, respectively, or FIG. 7i or i′, respectively or a portion thereof.

In an embodiment, the invention provides a nucleic acid molecule encoding the VLP of the invention, e.g., as shown in FIG. 1.

The nucleic acids of the invention may comprise nucleotide sequences and encode polypeptides (amino acid sequences) which are at least about 70% identical, preferably at least about 80% identical, more preferably at least about 90% identical and most preferably at least about 95% identical (e.g., 95%, 96%, 97%, 98%, 99%, 100%) to the reference nucleotide and amino acid sequences of the present invention (i.e., see examples herein) when the comparison is performed by a BLAST algorithm wherein the parameters of the algorithm are selected to give the largest match between the respective sequences over the entire length of the respective reference sequences. Polypeptides comprising amino acid sequences which are at least about 70% similar, preferably at least about 80% similar, more preferably at least about 90% similar and most preferably at least about 95% similar (e.g., 95%, 96%, 97%, 98%, 99%, 100%) to the reference amino acid sequences of the present invention when the comparison is performed with a BLAST algorithm wherein the parameters of the algorithm are selected to give the largest match between the respective sequences over the entire length of the respective reference sequences, are also included in the present invention.

The nucleic acid molecule may be a DNA molecule (e.g., an isolated cDNA) encoding the VLP of the invention. Additionally, the nucleic acid molecule may be a RNA (e.g., an isolated RNA such as isolated mRNA). Alternatively, the nucleic acid molecule may be a hybrid of cDNA and mRNA. For example, the invention provides for a DNA construct comprising a vector that expresses the VLP free of a viral genome of the invention.

The nucleic acid molecules of the invention also include derivative nucleic acid molecules which differ from DNA or RNA molecules. Derivative molecules include peptide nucleic acids (PNAs), and non-nucleic acid molecules including phosphorothioate, phosphotriester, phosphoramidate, and methylphosphonate molecules, that bind to single-stranded DNA or RNA in a base pair-dependent manner (Zamecnik, P. C., et al., 1978 Proc. Natl. Acad. Sci. 75:280284; Goodchild, P. C., et al., 1986 Proc. Natl. Acad. Sci. 83:4143-4146). Reviews of methods for synthesis of DNA, RNA, and their analogues can be found, e.g., in: Oligonucleotides and Analogues, eds. F. Eckstein, 1991, IRL Press, New York; Oligonucleotide Synthesis, ed. M. J. Gait, 1984, IRL Press, Oxford, England.

Additionally, the invention provides a vector which comprises the nucleic acid molecule of the invention. The term vector includes, but is not limited to, plasmids, cosmids, and phagemids. The host vector system comprises the vector of the invention in a suitable host cell. Examples of suitable host cells include but are not limited to bacterial cell and eukaryotic cells.

In one embodiment, the invention provides for a composition (e.g., pharmaceutical composition) comprising the VLP free of a viral genome of the invention in an effective immunizing amount and a suitable carrier, binders, diluents, adjuvants, excipients, and/or vehicles.

In one embodiment, the compositions of the invention further comprises a therapeutic agent admixed with the VLP. The therapeutic agent may be an anti-cancer agent which may be lenalidomide, ipilimumab, rituximab, alemtuzumab, ofatumumab, flavopiridol, Adriamycin, Dactinomycin, Bleomycin, Vinblastine, Cisplatin, ABT-199; acivicin; aclarubicin; acodazole hydrochloride; acronine; adozelesin; aldesleukin; altretamine; ambomycin; ametantrone acetate; amino glutethimide; amsacrine; anastrozole; anthramycin; asparaginase; asperlin; azacitidine; azetepa; azotomycin; batimastat; benzodepa; bicalutamide; bisantrene hydrochloride; bizelesin; bleomycin sulfate; brequinar sodium; bropirimine; busulfan; cactinomycin; calusterone; caracemide; carbetimer; carboplatin; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; ibrutinib; idelalisib; idarubicin hydrochloride; ifosfamide; ilmofosine; INCB-40093, IPI-145, IPI-443, iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; obinutuzumab; ormaplatin; oxisuran; pegaspargase; peliomycin; pentamustine; peplomycin sulfate; perfosfamide; pipobroman; piposulfan; piroxantrone hydrochloride; plicamycin; plomestane; porfmer sodium; porfiromycin; prednimustine; procarbazine hydrochloride; puromycin; puromycin hydrochloride; pyrazofurin; riboprine; rituximab; rogletimide; safingol; safingol hydrochloride; semustine; simtrazene; sparfosate sodium; sparsomycin; spirogerranium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfm; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

In another embodiment, the compositions of the invention further comprising a therapeutic agent admixed with the VLP and the therapeutic agent may be an alkylating agent which includes but are not limited to nitrogen mustards (e.g., bendamustine, mechloroethamine, cyclophosphamide, chlorambucil, melphalan), ethylenimine and methylmelamines (e.g., hexamethlymelamine, thiotepa), alkyl sulfonates (e.g., busulfan), nitrosoureas (e.g., carmustine, lomustine, semustine, streptozocin), or triazenes (decarbazine).

The invention further provides for a vaccine comprising the composition of the invention for inducing an immune response to the display polypeptides in a subject.

The invention also provides for an immunostimulatory composition for inducing an immune response in a subject comprising the VLP free of a viral genome of the invention. In an embodiment, the vaccine comprises the VLP free of a viral genome of the invention and an adjuvant. In another embodiment, the vaccine comprises a DNA vector that expresses the VLP free of a viral genome of the invention. In yet another embodiment, the vaccine comprises a viral gene delivery system to deliver a nucleic acid sequence that encodes the VLP free of a viral genome of the invention.

In further embodiments of the aspects of the invention, the Id antigen may be a recombinant antigen or a humanized antigen. In other embodiments, the Id antigen may be expressed and/or presented as single domain antibody, a diabody, an scFv, an scFv dimer, a dsFv, a (dsFv)₂, a dsFv-dsFv′, a Fv, a Fab, a Fab′, or a F(ab′)₂ fragment. In other embodiments, the fragment may be operably attached to a constant region, wherein the constant region is a kappa light chain, gamma-1 heavy chain, gamma-2 heavy chain, gamma-3 heavy chain or gamma-4 heavy chain.

In another embodiment, the invention provides a process comprising recovering a VLP of the invention from a culture medium.

In an embodiment, the invention further comprises administering a vaccine of the invention (a multivalent VLP of the invention). Administration includes, but is not limited to prior administration of the multivalent VLP of the invention followed by (at a pre-determined interval) administration of the vaccine of the invention so as, for example, to provide continuous long-term exposure of a cancer to therapeutic agents and, thereby, inhibit cancer growth.

According to embodiments of the invention, the degeneracy of the genetic code provides a predictable number of nucleic acid sequences encoding the multivalent VLP of the invention, the codons of which may be selected to optimally express the isolated nucleic acid in a host organism (including without limitation, bacteria, yeast, mammalian cells cultured in vitro, and cells of a mammal (including a human). Such expression is useful for production of the nucleic acid or the polypeptide in a host organism for subsequent isolation and use according to the invention or in cell free in vitro transcription and/or translation system.

In embodiments of the articles of manufacture of the invention, the article of manufacture comprises a multivalent VLP or composition of the invention.

In another embodiment, the invention provides an article of manufacture comprising a container and a composition of the invention contained therein, further comprising a package insert indicating that the composition can be used to treat or inhibit cancer, infection or an autoimmune disease.

Pharmaceutically acceptable carriers include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN™ 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Examples of binders include microcrystalline cellulose, gum tragacanth, glucose solution, acacia mucilage, gelatin solution, molasses, polvinylpyrrolidine, povidone, crospovidones, sucrose and starch paste. Diluents include, for example, lactose, sucrose, starch, kaolin, salt, mannitol and dicalcium phosphate.

### KITS OF THE INVENTION

According to another aspect of the invention, kits are provided. Kits according to the invention include package(s) comprising composition of the invention.

The phrase “package” means any vessel containing compositions presented herein. In preferred embodiments, the package can be a box or wrapping. Packaging materials for use in packaging pharmaceutical products are well known to those of skill in the art. Examples of pharmaceutical packaging materials include, but are not limited to, blister packs, bottles, tubes, inhalers, pumps, bags, vials, containers, syringes (including pre-filled syringes), bottles, and any packaging material suitable for a selected formulation and intended mode of administration and treatment.

The kit can also contain items that are not contained within the package but are attached to the outside of the package, for example, pipettes.

Kits may optionally contain instructions for administering compositions of the present invention to a subject having a condition in need of treatment. Kits may also comprise instructions for approved uses of components of the composition herein by regulatory agencies, such as the United States Food and Drug Administration. Kits may optionally contain labeling or product inserts for the present compositions. The package(s) and/or any product insert(s) may themselves be approved by regulatory agencies. The kits can include compositions in the solid phase or in a liquid phase (such as buffers provided) in a package. The kits also can include buffers for preparing solutions for conducting the methods, and pipettes for transferring liquids from one container to another.

The kit may optionally also contain one or more other compositions for use in combination therapies as described herein. In certain embodiments, the package(s) is a container for intravenous administration. In other embodiments, compositions are provided in an inhaler. In still other embodiments compositions are provided in a polymeric matrix or in the form of a liposome.

### METHODS OF THE INVENTION

The invention provides for a method for inhibiting tumor cells associated with a disease (supra.) or disorder in a subject. The method comprises obtaining a sample from the subject and identifying an Id antigen associated with a disease or disorder from the sample. A sample from the subject can be a cell, tissue (such as a tumor) or body fluid sample (such as blood). The method also comprises producing a recombinant Id antigen or fragment thereof and generating the VLP free of a viral genome of the invention which comprises the recombinant Id antigen or fragment thereof. Further, the method comprises administering an effective amount of the VLP free of a viral genome of the invention from step (d) to the subject so as to permit an immune response against the tumor cells.

The invention further provides for a method for inhibiting a disease or disorder in a subject. The method comprises obtaining a sample from the subject and identifying an Id antigen associated with the disease or disorder from the sample. The method also comprises producing a recombinant Id antigen or fragment thereof and generating the VLP free of a viral genome of the invention which comprises the recombinant Id antigen or fragment thereof. Further, the method comprises administering an effective amount of the VLP free of a viral genome of the invention from step (d) to the subject so as to permit an immune response against the tumor cells.

In another embodiment, the invention provides for a method of inhibiting tumor cells which comprises contacting the tumor cells with an effective amount of the composition of the invention.

The invention also provides for a method of treating, inhibiting or preventing the progression of a tumor in a subject, which comprises administering to said subject an effective amount of a multivalent VLP or composition of the invention. The multivalent VLP or composition may be administered intravenously, intramuscularly, subcutaneously, intraperitoneally, intranasally, intraocularly, intradermally, transmucosally or as an aerosol.

The invention further provides for a method of treating, inhibiting or preventing the progression of a disease or disorder comprising administering to said subject an effective amount of a multivalent VLP or composition of the invention.

In one embodiment, the disorder is an autoimmune disorder and may be a myasthenia gravis, chronic active hepatitis, primary biliary cirrhosis, dilated cardiomyopathy, myocarditis, dilated cardiomyopathy, autoimmune polyendocrine syndrome type I (APS-1), autoimmune hepatitis, cystic fibrosis vasculitidis, acquired hypoparathyroidism, Goodpasture syndrome, Crohn's disease, coronary artery disease, pemphigus foliaceus, pemphigus vulgaris, Guillain-Barr syndrome, type 1 diabetes, stiff man syndrome, Rasmussen encephalitis, autoimmune gastritis, Addison disease, insulin hypoglycemic syndrome (Hirata disease), type B insulin resistance, acanthosis, systemic lupus erythematosus (SLE), pernicious anemia, treatment-resistant Lyme arthritis, polyneuropathy, multiple sclerosis, demyelinating disease, rheumatic fever, atopic dermatitis, primary biliary cirrhosis. Graves' disease, neuromyelitis optica, autoimmune hypothyroidism, vitilago, autoimmune thyroiditis, autoimmune Hashimoto thyroiditis, celiac disease, and metastatic melanoma. In a preferred embodiment, the autoimmune disorder is Grave's disease. In another preferred embodiment, the autoimmune disorder is myasthenia gravis. In yet a further preferred embodiment, the autoimmune disorder is neuromyelitis optica.

In another embodiment, the disorder may be a systemic autoimmune disorder and may include ACTH deficiency, myositis, dermatomyositis, polymyositis, dermatomyositis, SLE, Sjogren syndrome, systemic sclerosis, rheumatoid arthritis (RA), progressive systemic sclerosis, systemic sclerosis, deimatomyositis, scleroderma, morphea, primary antiphospholipid syndrome, bullous pemphigoid, herpes gestationis, cicatricial pemphigoid, chronic idiopathic urticaria, necrotizing and cescentic glomerulonephritis (NCGN), system vasculitis, Wegener granulomatosis, Churg-Strauss syndrome, polymyositis, scleroderma, Raynaud syndrome, chronic liver disease, visceral leishmaniasis, and systemic autoimmune disease.

In yet another embodiment, the disorder may be a cancer or a paraneoplastic autoimmune disorder which may include neuropathy, small lung cell cancer, hepatocellular carcinoma, liver cancer, paraneoplastic pemphigus, paraneoplastic stiff man syndrome, paraneoplastic encephalomyelitis, sub-acute autonomic neuropathy, cancer, SLE, hepatocellular carcinoma, cancer-associated retinopathy, paraneoplastic opsoclonus myoclonus ataxia, lower motor neuron syndrome. Lambert-Eaton myasthenic syndrome, and paraneoplastic cerebellar degeneration.

In yet another embodiment, the disorder may be a solid tumor cancer which may be a adrenal cancer, anal cancer, aplastic anemia, bile duct cancer, bladder cancer, bone cancer, brain/CNS cancer, breast cancer, cancer of unknown primary origin, Castleman Disease, cervical cancer, colon/rectum cancer, endometrial cancer, esophagus cancer, Ewing family of tumors, eye cancer, gallbladder cancer, gastrointestinal carcinoid tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Liver Cancer, Lung Cancer, Lymphoma, Malignant Mesothelioma, Multiple Myeloma, Myelodysplastic Syndrome, Nasal Cavity and Paranasal Sinus Cancer, Nasopharyngeal Cancer. Neuroblastoma, Non-Hodgkin Lymphoma, Oral Cavity and Oropharyngeal Cancer, Osteosarcoma, Ovarian Cancer, pancreatic cancer, Penile Cancer, Pituitary Tumors, prostate cancer, Retinoblastoma, Rhabdomyosarcoma, Salivary Gland Cancer, Sarcoma, Skin Cancer. Stomach Cancer, Testicular Cancer, Thymus Cancer, Thyroid Cancer, Uterine Sarcoma, Vaginal Cancer. Vulvar Cancer, Waldenstrom Macroglobulinemia, Wilms Tumor, non-Hodgkin lymphoma, Hodgkin lymphoma, Burkitt's lymphoma, lymphoblastic lymphomas, mantle cell lymphoma (MCL), multiple myeloma (MM), small lymphocytic lymphoma (SLL), splenic marginal zone lymphoma, marginal zone lymphoma (extra-nodal or nodal), mixed cell type diffuse aggressive lymphomas of adults, large cell type diffuse aggressive lymphomas of adults, large cell immunoblastic diffuse aggressive lymphomas of adults, small non-cleaved cell diffuse aggressive lymphomas of adults, or follicular lymphoma.

In a further embodiment, the cancer may be any of head and neck cancer, breast, salivary gland, thyroid, pancreas, stomach, bladder, endometrial or uterine carcinoma, cervical cancer, ovarian, vulvar cancer, prostate, colon, rectal, colorectal, lung, non-small cell lung cancer, osteosarcoma, glioblastoma, kidney, liver, metastatic cancer. In a preferred embodiment, the cancer is a B-cell lymphoma (such as CLL). In another preferred embodiment, the cancer is a T-cell lymphoma. In yet a further preferred embodiment, the cancer is prostate cancer. In a further embodiment, the subject is a human, a farm animal, a horse, a dog, or a cat.

In another embodiment, the disorder may be a plasma protein autoimmune disorder or cytokine autoimmune disorder. Examples of plasma protein autoimmune disorder or cytokine autoimmune disorder include but not limited to autoimmune CI deficiency, SLE membrane proliferative glomerulonephritis, RA, systemic sclerosis, autoimmune thrombocytopenia purpura, immunodeficiency disorder, and atherosclerosis.

In another embodiment, the disorder may be a B-cell malignancy. Examples of B-cell malignancy include but not limited to non-Hodgkin lymphoma, Hodgkin lymphoma, chronic lymphocytic leukemia (CLL), mantle cell lymphoma and multiple myeloma, B-cell prolymphocytic leukemia, lymphoplasmocytic leukemia, splenic marginal zone lymphoma, marginal zone lymphoma (extra-nodal or nodal), plasma cell neoplasms (e.g., plasma cell myeloma, plasmacytoma, monoclonal immunoglobulin deposition diseases, heavy chain diseases), and follicular lymphoma (e.g., Grades 1, 11, 111 or IV).

In yet another embodiment, the disorder may be a T-cell malignancy. Examples of T-cell malignancy include but not limited to chronic lymphocytic leukemia (CLL), large granular lymphocyte leukemia (T gamma lymphoproliferative disease, mycosis fungoides/Sezary syndrome, diffuse aggressive lymphomas of adults, peripheral T-cell lymphomas (mixed cell type and large cell, immunoblastic), adult T-cell leukemia/lymphoma, angiocentric lymphomas (lymphomatoid granulomatosis polymorphic reticulosis, acute lymphocytic leukemia, or lymphoblastic lymphoma.

In one embodiment, the VLP is produced by a method for producing a population of icosahedral virus like particles free of a viral genome in a cell-free in vitro reaction. The method for producing a population of icosahedral virus like particles free of a viral genome in a cell-free in vitro reaction comprise synthesizing virus coat proteins in a prokaryotic cell-free in vitro translation reaction substantially free of polyethylene glycol and comprising a bacterial cell extract, components of polypeptide and/or mRNA synthesis machinery; a template for transcription for the translation of the polypeptide; monomers for synthesis of the polypeptide; and co-factors, enzymes and other reagents necessary for translation to produce at least about 250 ug/ml of the virus coat proteins-under conditions permissive for the virus coat proteins to self-assemble into a stable icosahedral virus like particle free of a viral genome, and comprising at least 60 separate proteins.

In an embodiment, the invention provides a method of treating a cancer in a subject further comprising administering to the subject a therapeutically effective amount of one or more chemotherapeutic agents, wherein the chemotherapeutic agents are one or more of the following: alkylating agents; thiotepa; cyclosphosphamide; alkyl sulfonates; busulfan; improsulfan; piposulfan; aziridines; benzodopa; carboquone; meturedopa; uredopa; ethylenimines; methylamelamines; altretamine; triethylenemelamine; trietylenephosphoramide; triethylenethiophosphaoramide; trimethylolomelamine; nitrogen mustards; chlorambucil; chlornaphazine; cholophosphamide; estramustine; ifosfamide; mechlorethamine; mechlorethamine oxide hydrochloride; melphalan; novembichin; phenesterine; prednimustine; trofosfamide; uracil mustard; nitrosureas; carmustine; chlorozotocin; fotemustine; lomustine; nimustine; ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; methotrexate; 5-fluorouracil; denopterin, methotrexate, pteropterin, trimetrexate; fludarabine, 6-mercaptopurine, thiamiprine, thioguanine; ancitabine, azacitidine, 6-azauridine, carmofur, cytarabine, dideoxyuridine, doxifluridine, enocitabine, floxuridine, 5-FU; calusterone, dromostanolone propionate, epitiostanol, mepitiostane, testolactone; aminoglutethimide, mitotane, trilostane; frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide K (PSK); razoxane; sizofiran; spirogermanium; tenuazonic acid; triaziquone; 2, 2′,2″-trichlorotriethylamine urethan; vindesine; dacarbazine; mannomustine; mitobronitol; mitolactol; pipobroman; gacytosine; arabinoside; cyclophosphamide; thiotepa; paclitaxel; docetaxel; chlorambucil; gemcitabine; 6-thioguanine; mercaptopurine; methotrexate; cisplatin; carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor 9-nitrocamptothecin; difluoromethylornithine; retinoic acid; esperamicins; capecitabine; tamoxifen; raloxifene; aromatase inhibiting 4(5)-imidazoles; 4-hydroxytamoxifen; trioxifene, keoxifene; LY117018; onapristone; toremifene; flutamide; nilutamide; bicalutamide; leuprolide; and goserelin.

In yet another embodiment, the disorder is an infectious disease and may be polio, respiratory syncytial virus (RSV) infection AIDS, hepatitis B, hepatitis C, hepatitis E, rabies, herpes, HSV, EBV, influenza, smallpox, myxoma infection, rhinovirus infection, coronavirus infection, whooping cough (rubella virus infection), adenovirus infection, papilloma virus infection or human T-cell leukemia virus (HTLV) infection. In a preferred embodiment, the infectious disease is HIV. In another preferred embodiment, the infectious disease is influenza. In yet a further preferred embodiment, the infectious disease is RSV infection.

The invention also provides for a method for producing a VLP free of a viral genome protein comprising culturing the host vector system the invention under suitable culture conditions so as to produce the VLP free of a viral genome in the host and recovering the VLP free of a viral genome so produced. Alternatively, the VLP of the invention may be produced in a cell free in vitro transcription and/or translation system (Bundy 2008b, Bundy 2011).

In one embodiment, the VLP free of a viral genome is produced by the method of the invention and may contain at least one unnatural amino acid (also referred to herein as non-natural amino acid or nnAA) used to conjugate it to a display polypeptide (supra.).

For attachment (also referred to herein as conjugation) of the display agents to the VLP, the virus coat polypeptides of the VLP may be modified to comprise at least one first unnatural amino acid (also referred to herein as non-natural amino acid or non-canonical amino acid (nnAA)) at a site of interest and the two or more display polypeptides may be modified to comprise at least one second unnatural amino acid, wherein the first unnatural amino acid is different from, and reactive with the second unnatural amino acid (supra.). An example of one first unnatural amino acid is azidohomoalanine. An example of a second unnatural amino acid is propargyloxyphenylalanine. The azide functional group of azidohomoalanine incorporated into a capsid protein of a VLP may participate in a (3+2) cycloaddition click reaction with an alkyne functional group of propargyloxyphenylalanine incorporated into a display agent, resulting in VLP crosslinked to a display agent. Other unnatural amino acid-containing capsid proteins within the same VLP may similarly participate in the (3+2) cycloaddition click reaction to produce a VLP with two or more display agents. In another embodiment, the VLP may display a polypeptide and a CpG. In another embodiment, the VLP may display a polypeptide and a nucleic acid or a modified nucleic acid. In another embodiment, the VLP may display two or more polypeptides and a CpG. In a separate embodiment, the VLP may display two or more polypeptides and a nucleic acid or a modified nucleic acid.

The following examples are provided to further illustrate aspects of the invention. These examples are non-limiting and should not be construed as limiting any aspect of the invention.

### EXAMPLES

### Example 1

### In Vivo Studies

38C13 was selected as a model for the study of the therapeutic efficacy of the VLP vaccines in a cancer model. (Bergman 1977, Betting 2008, Haimovich 1999. Kim 1979) A total of 109 Female C3H/HeN mice, 6 weeks old, were purchased from Charles River Laboratories and housed in a temperature-controlled room with a 12-hour light/dark cycle, with ad libitum access to food and water throughout the study. All animal study protocols were approved by IACUC to their guidelines. The number of animals and treatment groups are shown in Table 1.

TABLE 1
38C13 Vaccine Study Groups

| | | | | Humoral | T Cell |
|---|---|---|---|---|---|
| | | | | Immune | Immune |
| | | | Mice | Response | Response |
| | | | Vacci- | Analysis: | Analysis: |
| | | | nated | Post vacci- | Post vacci- |
| | | BB | (Chal- | nation (Post | nation (Post |
| Group | Vaccine | Number | lenged) | challenge) | challenge) |
| 1 | 38CIgM- | | 12 (10) | + (+) | + (+) |
| | KLH | | | | |
| 2 | 38Cs-Fusion | | 12 (10) | + (+) | + (+) |
| 3 | VLP | | 22 (19) | + (+) | + (+) |
| 4 | 38Cf60-F10- | BB-005 | 12 (10) | + (+) | + (+) |
| | C20-mG20- | | | | |
| | VLP | | | | |
| 5 | 38Cs70-F10- | BB-004 | 10 (10) | + (+) | N/A (+) |
| | C20-mG20- | | | | |
| | VLP | | | | |
| 6 | 38Cs90-F10- | BB-003 | 10 (10) | + (+) | N/A (+) |
| | C20-VLP | | | | |
| 7 | 38Cs100- | BB-002 | 12 (10) | + (+) | + (+) |
| | mG20-VLP | | | | |
| 8 | 38Cs100- | BB-001 | 12 (10) | + (+) | + (+) |
| | C20-VLP | | | | |
| 9 | 38CIgM50- | | 2 (0) | + (N/A) | + (N/A) |
| | C20-VLP | | | | |

Immunization

Vaccines were constructed as described in Example 2 and stored in aliquots at −80° C. Immediately prior to administration vaccines were thawed and diluted to a final concentration of 81 pico moles of Id heavy chain variable region per 200 microliters buffer (PBS containing 0.05% Tween-20). Mice were immunized a total of 3 times (D 1, 10, and 20) at 10 days intervals by subcutaneous injection of 100 microliters in each flank.

Serum Collection

Immune sera were collected from 3 mice per group the day before (pre-bleed; D-1), 1 week after the 2^{nd} and 3^{rd} immunizations (D 17 and 27) and at the study endpoint and stored at −20° C. Sera were also collected from the terminal blood for each animal at the study endpoint.

Anti-Id Immune Response Monitoring

Anti-Id humoral immune response was measured in mouse sera using a solid phase ELISA-based assay. (Milner 2007) Briefly, test wells of microtiter plates were coated with the 38C13 Id used to immunize the animal group or HBC. Serum dilutions were prepared and allowed to interact with the plates. Anti-mouse Ig reagents were used for detection. An estimate of anti-Id antibody titer was made by referencing to signals generated from a spike-in mouse anti-38C13Id antibody in naïve mouse serum.

Tumor Challenge

Fourteen days after the final vaccination (D 34), mice were challenged subcutaneously with 38C13 murine B cell lymphoma. 38C13 cells were resurrected 5 days before tumor challenge, and the cell culture were passaged on the day 3 and 4 culture before use. Four hundred cells in 100 microliters of incomplete RPMI media were subcutaneously implanted to the right lower flank of each animal. This number of cells had previously been determined to able to produce tumors of approximately 4000 cubic millimeters in naïve animals within an approximately 20 day period. Once tumors were established, they were measured every day, and the tumor volume were approximated using the ellipsoidal formula: length×width×height×0.52 (in cubic millimeters). Animals were euthanized and tumors with or without spleen were harvested when subcutaneous tumors measured more than 4000 mm³ or until any mouse appeared to be moribund.

Veterinary Observations and Body Weight Analysis.

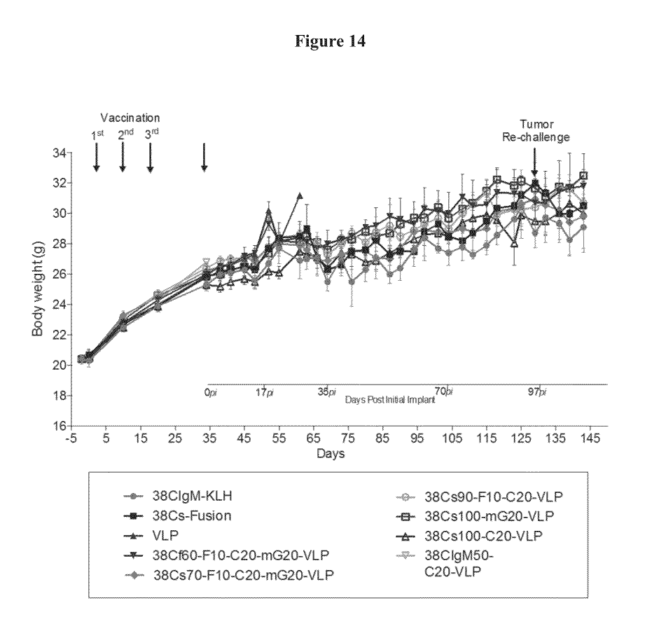

There were no adverse effects upon either vaccine administrations or tumor challenge in all animals during the study. Animals were active with minimal toxicity until right before the implanted tumors were close to the end point in size. Mean body weights for each treatment groups are shown in FIG. 14. Body weights of all animals were constantly gaining during the vaccination or after challenged with tumor. Acute body weight gains in some tumor bearing animals were also associated with the size of the tumor they developed.

At the end point, some animals that were unprotected became acutely moribund with various degrees of tumor metastasis. Some had purulent and hemorrhagic ascites or pleural effusion, metastasis in peripheral lymph nodes, or at various locations in the abdominal cavity and thoracic cavity. A few animals were also found dead toward the end point.

Tumor Protection Associated with Immunization.

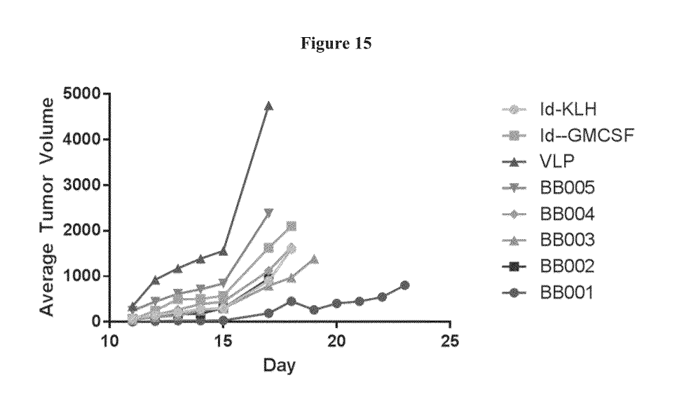

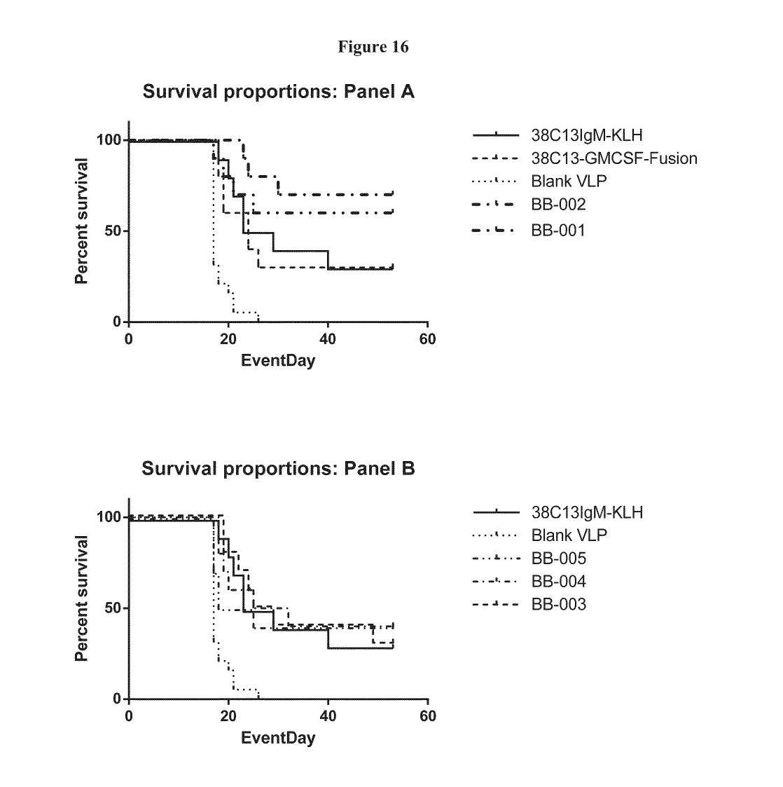

The various vaccine constructs were compared for their efficacy in inducing protection against tumor challenge. Mean (±SEM) values calculated from the tumor volumes of 10 mice per treatment group except for group 3 (control), which consists of 19 mice. Drug efficacy was expressed as the percentage tumor growth inhibition (TGI %), calculated using the equation 100-((T−C)/C*100), where T is the mean tumor of the treated tumor and C is the mean tumor of the control group (VLP) at the time of mean tumor volume in the control group reached to the end point. The control mice (injection of VLP) in group 3 did not show any protection; all mice developed subcutaneous tumors within day 13 pi, and tumor volume reached to the end point for all animals by day 27 pi (FIG. 15). In contrast, mice immunized with vaccine constructs suppressed tumor growth at various degrees, and resulted in 50-96% tumor growth inhibition on 30 day 17 pi, when average tumor volume of the control (VLP) group had reached to the end point tumor volume (FIG. 15). Compared with the VLP control group (group 3), 38Cs100-C20-VLP (group 8) immunization resulted in 96% tumor growth inhibition on day 17 pi. Likewise, 38Cs100-mG20-VLP (group 7) resulted in 80% tumor growth inhibition (day 17 pi). Tumors from the mice immunized with vaccine constructs also achieved longer time to endpoint (TTE) compare to the TTE of control groups including tumor-free-survivors as can be seen in FIG. 16 and Table 2. By day 35 pi, all vaccine constructs protected with 30-70% long term tumor-free-survivors, where “38Cs100-C20-VLP” (group 8) achieved the maximum success (70%, 7 of 10 mice) and “38Cs-Fusion” (group 2) and “38Cs70-F1-C20-mG20-VLP” (group 5) achieved the minimal complete protection (30%, 3 of 10 mice) (Table 2). The percentage of tumor-free-survivors in each group was recorded for 97 days pi until mice were re-challenged with 38C13 cells.

TABLE 2
Number of tumor-free survivors.

| | | Survival | Median Days |
|---|---|---|---|
| Group | Vaccine | Proportion % | to Event |
| 1 | 38CIgM-KLH | 30 | 26 |
| 2 | 38Cs-Fusion | 30 | 24.5 |
| 3 | VLP | 0 | 17 |
| 4 | 38Cf60-F10-C20-mG20-VLP | 40 | 21.5 |
| 5 | 38Cs70-F10-C20-mG20-VLP | 40 | 28.5 |
| 6 | 38Cs90-F10-C20-VLP | 30 | 27 |
| 7 | 38Cs100-mG20-VLP | 60 | Undefined |
| 8 | 38Cs100-C20-VLP | 70 | Undefined |

Immune Response Results

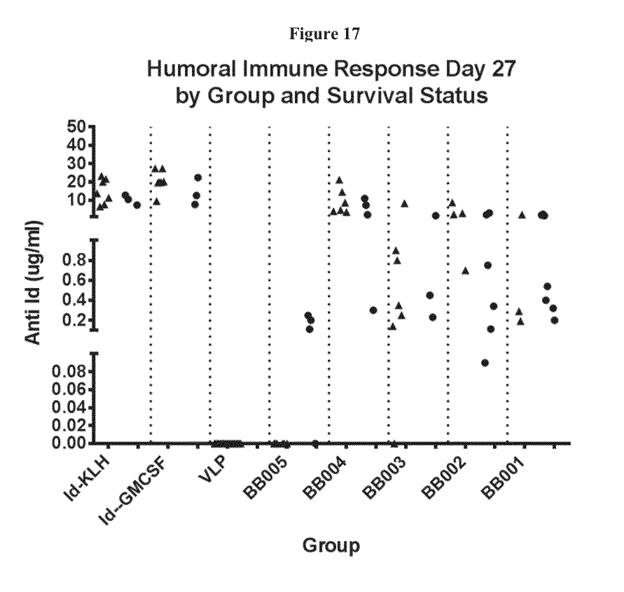

Anti-Id immune response results are shown in FIG. 17. All animals in the positive control groups achieve anti-Id antibody titers measured at over 1 microgram per milliliter. No anti-Id response was seen in the negative control group. For groups 4 to 8, animals given VLPs with Id and other components attached, antibody titers varied, but were generally lower than that observed for the positive controls.

### SUMMARY

VLP groups generally outperformed the positive control vaccines despite generally lower immune response in terms of anti-Id antibody titer and slower onset of immune response. This result was unexpected and points to the complex interplay of immune response and therapeutic efficacy for complex diseases such as cancer.

### Example 2

### Production of VLP Vaccines

Engineering Components

The Hepatitis B virus (HepB) is an enveloped DNA virus. A mutant truncated form of its capsid-forming Hepatitis B core antigen (HBC) has been found to self-assemble in the right conditions to form a 240mer icosahedral VLP (Zlotnick 1996). The VLPs contain no DNA, are noninfectious, and stable over wide ranges of pH and temperature. The HBC VLP's surface is decorated with an ordered array of projecting alpha helices which can be exploited for successful foreign antigen and immunostimulant display in vaccine development (Pumpens 2001). The physical and chemical properties of HBC VLPs synthesized in CFPS have been well characterized, including sizing by transmission electron microscopy and are suitable for pharmaceutical development (Bundy 2008, Bundy 2010, Bundy 2011, Kanter 2007, Voloshin 2005, Yang 2004).

All template sequences were designed and optimized for reduced secondary structure using Mfold software (Zuker 2003) and optimized bacterial codon usage for expression. For development, all proteins except the HBC have been tagged for purification with hexahistidine, Strep-tag or FLAG-tag sequences. These purification tags may be removed prior to human trials as needed. Templates were synthesized de-novo and cloned into plasmid vectors (pY71 or PET). Each construct was sequence verified.

Hep B Core Protein Production and Purification of VLP

We produced VLPs with nnAAs incorporated at specific sites such that proteins and other molecules can be attached to the VLP with e.g. click chemistry. The Hepatitis B virus (HVB) is an enveloped DNA virus; we use a truncated form of its capsid-forming core antigen that self-assembles when expressed in CFPS to form a 240mer (T=4), icosahedral VLP (Zlotnick 1996). The VLPs are noninfectious and very stable over wide ranges of pH and temperature. (Bundy 2008). HVB core antigen produced in 20 to 40 microliter reactions yielded over 400 micrograms per milliliter, and the majority of the total synthesized polypeptide was soluble. The HVB VLP's surface is characterized by an ordered array of projecting alpha helices which can be exploited for successful attachment of antigens and immunostimulants in vaccine development (Pumpens 2001).

Cell-free protein synthesis (CFPS) reactions for HepB Core (HBC) with azidohomoalanine incorporation have been described previously. (Bundy 2008b, Bundy 2009, Bundy 2011, Patel 2011) Reactions were performed in 10 ml reaction volume in two T75 plates (5 ml per each plate) and incubated for 16 hours at 30° C. The reaction contained 8 mM magnesium glutamate, 10 mM ammonium glutamate, 130 mM sodium glutamate, 35 mM sodium pyruvate, 1.2 mM AMP, 0.86 mM each of GMP, UMP, and CMP, 2 mM amino acids minus methionine, 2 mM azidohomoalanine (MedChem Co), 4 mM sodium oxalate, 1 mM putrescine, 1.5 mM spermidine, 15 mM potassium phosphate, 100 nM T7 RNA polymerase, and 500 μg plasmid DNA template, and 3 ml cell-free extract.

After incubation, the reaction products were centrifuged for 15 minutes at 15.000 g to remove the aggregates. The supernatant was combined with saturated ammonium sulfate to the final 30% saturation. The sample was mixed for an additional hour, then the sample are centrifuged to pellet the precipitate.

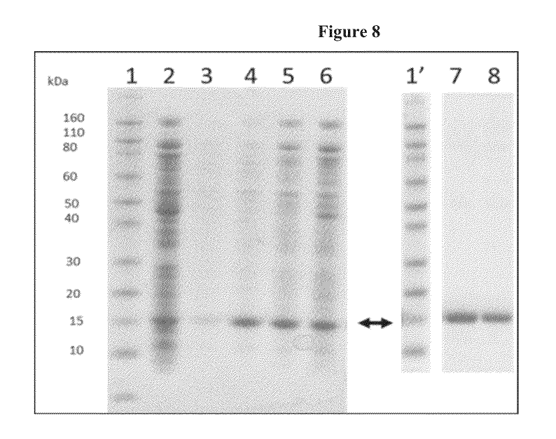

HBC VLP was purified by size exclusion using Sepharose 6 Fast Flow (GE Life Technologies). The ammonium sulfate precipitate was resuspended in 1 ml 50 mM Tris pH7.5/500 mM NaCl, and loaded onto a Sepharose 6 Fast Flow column (2.5 cm id×25 cm length) pre-equilibrated with the same buffer. The column was run at a flow rate of 0.5 mL/min. The fractions were collected and analyzed by SDS-PAGE. HepB VLP was well separated from the aggregates and smaller sized proteins. The yield in this example was 4 mg from the 10 ml reaction volume. Representative results are shown in FIG. 8.

Component Production & Purification

Proteins were synthesized using CFPS in cell-free extract containing the translation machinery and enriched with a cocktail of ribonucleotide-triphosphates, T7 RNA polymerase, amino acids and NAD. Addition of the proper DNA sequence results in high-yield protein synthesis. To enable bio-conjugation of proteins through click chemistry (a Cu(I)-catalyzed [3+2]cycloaddition) to the azide-containing underivatized VLP, a nnAA with either an alkyne residue is incorporated at specific sites (Bundy 2010, Patel 2011). Each component protein was purified through affinity purification, size separation, ion exchange or other methods for purification as appropriate. (Bundy 2010. Goerke 2009, Kanter 2007, Patel 2010, Patel 2011).

Production of Flagellin-T240X

CFPS reactions for flagellin-T240X were performed in a 10 mL reaction volume split into 5 ml in each of two 500 ml conical centrifuge tube and incubated for 16 hours at 30° C. on a nutator. The reaction contains 8 mM magnesium glutamate, 10 mM ammonium glutamate, 130 mM potassium glutamate, 35 mM sodium pyruvate, 1.2 mM AMP, 0.86 mM each of GMP. UMP, and CMP, 2 mM standard proteinogenic amino acids, 2 mM propargyloxyphenylalanine, 4 mM potassium oxalate, 1 mM putrescine, 1.5 mM spermidine, 15 mM potassium phosphate, 100 ug/ml T7 RNA polymerase, 150 μg flagellin-T240X plasmid DNA template, 4.8 mg MJTyRS^{pPa}, 60 ug otRNA and 3 ml bacterial cell-free extract.

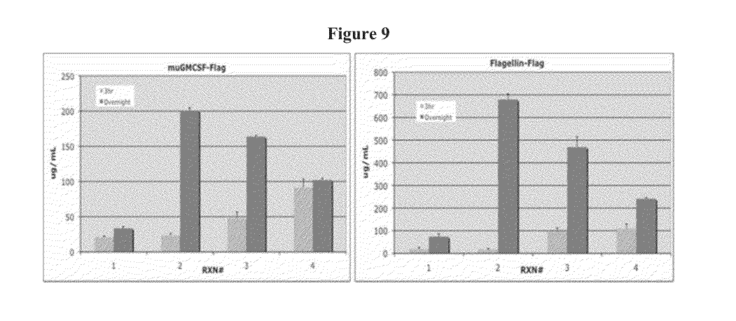

After incubation, the reaction products were centrifuged for 15 minutes at 15,000 g to remove the aggregates. The supernatant was loaded onto anti-FLAG resin and washed with TBS buffer 6 times. The product was eluted with 100 ug/ml Flag-peptide. The product was analyzed by SDS-PAGE gel electrophoresis and the protein was stored at −80° C. Representative results are shown in FIG. 9.

Production of huGM-CSF-T95Y, muGM-CSF-T92x and IM9-S27X-38C13scFvId Fusion Proteins

CFPS reactions for huGM-CSF-T95X, muGM-CSF-T92x and IM9-S27X-38C13scFvId fusion proteins were performed in a 10 mL reaction volume split into 5 ml in each of two 500 ml conical centrifuge tube and incubated for 16 hours at 30° C. on a nutator. The reaction contains 8 mM magnesium glutamate, 10 mM ammonium glutamate, 130 mM potassium glutamate, 35 mM sodium pyruvate, 1.2 mM AMP, 0.86 mM each of GMP, UMP, and CMP, 2 mM standard proteinogenic amino acids, 2 mM propargyloxyphenylalanine, 4 mM potassium oxalate, 1 mM putrescine, 1.5 mM spermidine, 15 mM potassium phosphate, 100 ug/ml T7 RNA polymerase, 1 mM reduced glutathione, 4 mM oxidized glutathione, 2 mM E. coli disulfide isomerase DsbC, 150 μg appropriate plasmid DNA template, 4.8 mg Mj-tyrosyl-tRNA (MjtRNA) synthease (MJTyRS^{pPa}), 60 ug otRNA template, and 3 ml bacterial cell-free extract. Cell-free extracts were treated with 50 μM iodoacetamide (IAA) for 20 minutes at room temperature before adding to the mixture.

After incubation, the reaction products was centrifuged for 15 minutes at 15,000 g to remove the aggregates. The supernatant was loaded onto anti-FLAG or anti-STREP resin and washed with TBS buffer 6 times. The product was eluted with 100 ug/ml Flag-or Strep-peptides. Proteins were analyzed by SDS-PAGE gel electrophoresis and stored at −80° C. Representative results are shown in FIG. 9.

Production of 38C13 IgM and 38C13 F(ab′)2

38C13 IgM producing cell line was obtained from Dr. Ron Levy of Stanford University. (Bergman 1977. Bergman 1977, Eshhar 1979, Maloney 1985) Cells were expanded using standard cell culture conditions and antibody was purified using antibody constant region affinity chromatography. Products were analyzed by reducing SDS-PAGE was used to analyze for purity.

F(ab′)2 was prepared from the IgM by partial digestion using partial reduction of the IgM and partial digestion of the constant regions. SDS-PAGE was used to analyze for purity.

Production of CpG-X

A CpG sequence shown in FIG. 5 was manufactured and assayed by mass spectroscopy and HPLC by Sigma Aldrich.

Alkyne-Azide “Click” Conjugation for VLP Component Assembly

To enable bio-conjugation of proteins through ‘click chemistry’ (a Cu(I)-catalyzed [3+2]cycloaddition), an unnatural amino acid with either an alkyne or an azide residue is incorporated at specific sites (Bundy 2010, Patel 2011). Each component protein is purified through affinity purification, size separation and ion exchange as appropriate. (Bundy 2010, Goerke 2009, Kanter 2007, Patel 2010, Patel 2011). We have demonstrated click chemistry for the attachment of a alkyne derivatized CpG sequence, muGM-CSF, huGM-CSF, scFV Id (38C13 model) and flagellin.

The azide-alkyne click reactions were performed in a humidified argon-sparged reaction vessel that maintained the reduced state of the 1 mM tetrakis(acetonitrile)-copper(I)hexafluorophosphate catalyst ([(CH3CN)4Cu]PF6)(Sigma Aldrich). The reaction contained the VLP-azide and one or more of the alkyne derivatized components at desired concentrations. The reaction also contained 0.5 mM tris(triazolylmethyl) amine Cu ligand (TTMA) enhancer (Zhou 2004), phosphate buffered saline and optionally sodium ascorbate at 200 uM. The reactions were carried out at 37 degrees for approximately 16 hours. The assembled VLPs were purified by size exclusion chromatography and optionally further by re-precipitation of the assembled VLPs in ammonium sulfate 30% and subsequent resuspension. Endotoxin was removed by phase separation using Triton X-114.

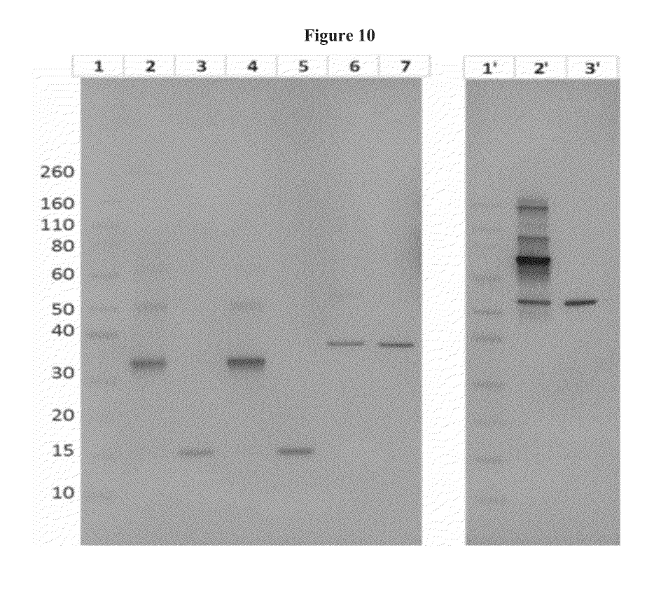

Specifically, for production of single component VLPS, 100 ug of flagellin-T240x, huGM-CSF-T95X, muGM-CSF-T92X, or ScFV-IM9-X, 60 ug Hep B Core VLP, 0.5 mM TTMA, 1 mM Tetrakis Cu(I), 200 uM sodium ascorbate were prepared in 130 ul total volume of phosphate buffered saline. The reaction was allowed to proceed for 16 hours at 37 degrees in a humidified argon sparged chamber. Products were analyzed for conjugation by SDS-PAGE and Western blot. Representative results are shown in FIG. 10.

Multi-component vaccines were produced by mixing the components and VLP at defined ratios prior to addition of the TTMA and Tetrakis Cu(T). The ratios used to make vaccines for the mouse study described in Example 1 are shown in Table 3.

The strained-alkyne maleimide linker (Life Technologies C-10413) was used to attach 38C13 IgM and 38C13 F(ab′)2 to the VLP. F(ab′)2 fragments obtained from 38C13 IgM producing cell lines were prepared by partial digestion of the constant region. After using an approach described for partial reduction of hinge-region disulfides, the linker was reacted to free sulfhydryls of the F(ab′)2 or IgM, especially those made available in the hinge region. The strained-alkyne was then used for attachment to the free-azide group of the VLP using the buffer conditions described for “Click” conjugation above with or without the Copper catalyst and TTMA enhancer.

TABLE 3
Recipes for Vaccine Production.

| | Vaccine | | | | 2-SU 38C13 | | | 5-huGM- | 5-muGM- |
|---|---|---|---|---|---|---|---|---|---|
| | Molecular | 1-HBC | 2-38C13 | 2-38C13 | ScFV-IM9 | 3-Flagellin | 4-CpG | CSF | CSF |
| VaccineAbbr | Weight (kDa) | (mg) | IgM (mg) | F(ab′)2 (mg) | (mg) | (mg) | (mg) | (mg) | (mg) |
| 38ClgM50-C20-VLP | 13829.2 | 0.22 | 0.66 | [symbol] | [symbol] | [symbol] | 0.017 | [symbol] | [symbol] |
| 38Cf60-F10-C20-mG20-VLP | 12200.7 | 0.25 | [symbol] | 0.57 | [symbol] | 0.041 | 0.019 | [symbol] | 0.025 |
| 38Cs70-F10-C20-mG20-VLP | 7590.7 | 0.32 | [symbol] | [symbol] | 0.26 | 0.053 | 0.024 | [symbol] | 0.032 |
| 38Cs90-F10-C20-VLP | 8019.1 | 0.31 | [symbol] | [symbol] | 0.32 | 0.05 | 0.023 | [symbol] | [symbol] |
| 38Cs100-mG20-VLP | 8029.2 | 0.23 | [symbol] | [symbol] | 0.27 | [symbol] | [symbol] | [symbol] | 0.023 |
| 38Cs100-C20-VLP | 7867.6 | 0.24 | [symbol] | [symbol] | 0.27 | [symbol] | 0.018 | [symbol] | [symbol] |
| VLP | 4017.6 | 1.1 | [symbol] | [symbol] | [symbol] | [symbol] | [symbol] | [symbol] | [symbol] |

[symbol]

indicates data missing or illegible when filed

Production of 38C13scFv-1M9-GM-CSF Control Protein

One control protein for the mouse study of Example 1, the fusion protein 38C13ScFV-1M9-GM-CSF, does not have nnAAs incorporated. This protein was produced by CFPS according to published methods. (Yang 2005)

Assays to Test for Component Activity

Each of the immunostimulant components were tested for activity in either a binding assay using the ForteBio instrument (cytokines and 38C13-containing) or a cell-based reporter (flagellin and CpG sequence). Representative data follows for the murine IL-15 and flagellin assays. An assay for activity of the azide-VLP has also been developed.

ForteBio Binding Assay

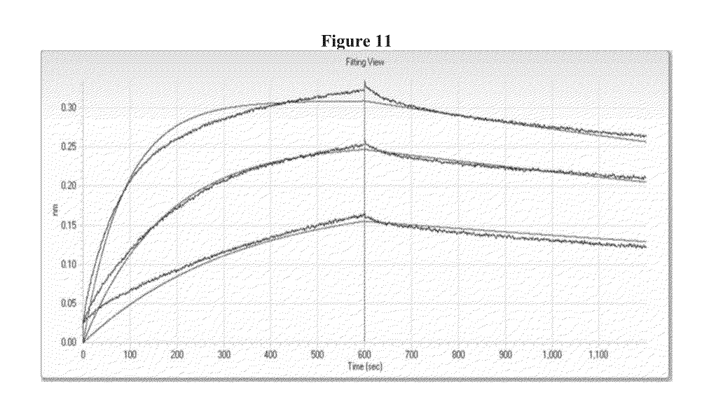

Purified recombinant rMuIL15Ra (murine IL-15 receptor R&D Systems) was solubilized in PBS at a final concentration of 1 mg/ml and biotinylated with EZ-Link NHS-LC-Biotin (Thermo Scientific). Biotinylation was carried out at room temperature for 2 hours and then dialyzed overnight in PBS. The biotinylated reagent was stored at 4° C. at a concentration of 0.1 mg/ml. ForteBio SA Biosensors were pre-hydrated in 200 μl of IX kinetic buffer for 10 minutes in a black 96 well plate. One ml of biotinylated rMuIL15Ra (murine IL-15 control) was prepared at a final concentration of 5 μg/ml in IX kinetic buffer or PBS. rMuIL15 (R&D Systems) was titrated 2 fold starting at 200 nM for 3 additional dilutions with final volumes of 200 μl each. The calculated on-rate constant is 5.53 e −4 M⁻¹ sec⁻¹; off-rate constant is 3.08 e4 sec⁻¹ and the dissociation constant is 5.57 e-9 M. The kinetic curves are shown in FIG. 11.

HEK-Blue TLR-5 and TLR-9 Reporter Cell Assay

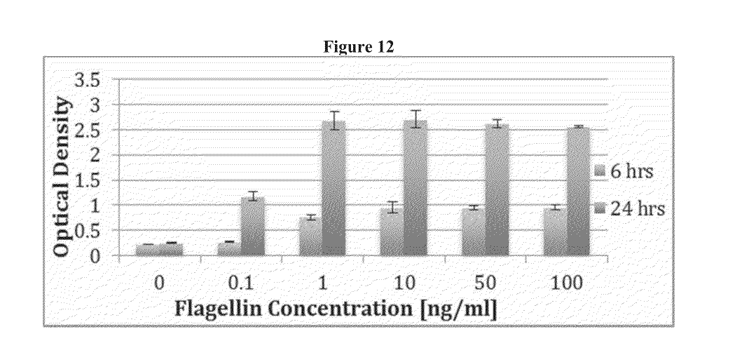

The commercially available InvivoGen HEK-Blue™ cell based assays have been implemented to analyze flagellin and CpG (InvivoGen hkb-htlr5, hkb-mtlr5, hkb-htlr9 and hkb-mtlr9). Cells expressing human or mouse TLR5 or TLR9 have shown success in demonstrating activity of flagellin and CpG respectively. The assay has been implemented to analyze flagellin as shown in FIG. 12.

Azide VLP Activity Assay

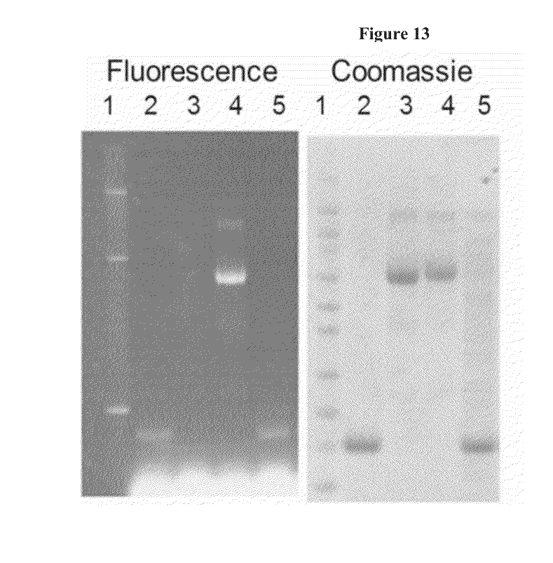

Purified HepBc VLP was reacted with DyLight-488-phosphine (Invitrogen) in PBS solution. BSA-azide control was prepared by reacting BSA with 4 mM azido-succimide (Invitrogen) and purified using a desalting column. The reaction products were analyzed on reducing SDS-PAGE. Prior to staining with Coomassie (Invitrogen) a fluorescence image of the gel was generated. Representative data are shown in FIG. 13.

### REFERENCES

Ai, W. Z., R. Tibshirani, B. Taidi, D. Czerwinski and R. Levy (2009). “Anti-idiotype antibody response after vaccination correlates with better overall survival in follicular lymphoma.” Blood113(23): 5743-5746.
Basith, S., B. Manavalan, G. Lee, S. G. Kim and S. Choi (2011). “Toll-like receptor modulators: a patent review (2006-2010).” Expert Opin Ther Pat 21(6): 927-944.
Bendandi, M. (2009). “Idiotype vaccines for lymphoma: proof-of-principles and clinical trial failures.” Nat Rev Cancer 9(9): 675-681.
Bendandi, M., C. D. Gocke, C. B. Kobrin, F. A. Benko, L. A. Sternas, R. Pennington, T. M. Watson, C. W. Reynolds, B. L. Gause, P. L. Duffey, E. S. Jaffe, S. P. Creekmore, D. L. Longo and L. W. Kwak (1999). “Complete molecular remissions induced by patient-specific vaccination plus granulocyte-monocyte colony-stimulating factor against lymphoma.” Nat Med 5(10): 1171-1177.
Bergman, Y. and J. Haimovich (1977). “Characterization of a carcinogen-induced murine B lymphocyte cell line of C3HieB origin.” Eur J Immunol 7(7): 413-417.
Bergman, Y., J. Haimovich and F. Melchers (1977). “An IgM-producing tumor with biochemical characteristics of a small B lymphocyte.” Eur J Immunol 7(8): 574-579.
Betting, D. J., K. Kafi, A. Abdollahi-Fard, S. A. Hurvitz and J. M. Timmerman (2008). “Sulfhydryl-based tumor antigen-carrier protein conjugates stimulate superior antitumor immunity against B cell lymphomas.” J Immunol 181(6): 4131-4140.
Bird, R. E., K. D. Hardman, J. W. Jacobson, S. Johnson, B. M. Kaufman, S. M. Lee, T. Lee, S. H. Pope, G. S. Riordan and M. Whitlow (1988). “Single-chain antigen-binding proteins.” Science242(4877): 423-426.
Bundy, B. C., M. J. Franciszkowicz and J. R. Swartz (2008). “Escherichia coli-based cell-free synthesis of virus-like particles.” Biotechnol Bioeng 100(1): 28-37.
Bundy, B. C., A. R. Goerke and J. C. Swartz (2008b). WO2008/066583.
Bundy, B. C. and J. C. Swartz (2009). US2009/0317861.
Bundy, B. C. and J. R. Swartz (2010). “Site-specific incorporation of p-propargyloxyphenylalanine in a cell-free environment for direct protein-protein click conjugation.” Bioconjug Chem 21(2): 255-263.
Bundy, B. C. and J. R. Swartz (2011). “Efficient disulfide bond formation in virus-like particles.” J Biotechnol 154(4): 230-239.
Cooper, G. S., M. L. Bynum and E. C. Somers (2009). “Recent insights in the epidemiology of autoimmune diseases: improved prevalence estimates and understanding of clustering of diseases.” J Autoimmun 33(3-4): 197-207.
Eshhar, Z., C. Blatt, Y. Bergman and J. Heimovich (1979). “Induction of secretion of IgM from cells of the B cell line 38c-13 by somatic cell hybridization.” J Immunol 122(6): 2430-2434.
Fontoura. P., H. Garren and L. Steinman (2005). “Antigen-specific therapies in multiple sclerosis: going beyond proteins and peptides.” Int Rev Immunol 24(5-6): 415-446.
Goerke, A. R. and J. R. Swartz (2009). “High-level cell-free synthesis yields of proteins containing site-specific non-natural amino acids.” Biotechnol Bioeng 102(2): 400-416.
Haimovich, J., T. Kukulansky, B. Weissman and N. Hollander (1999). “Rejection of tumors of the B cell lineage by idiotype-vaccinated mice.” Cancer Immunol Immunother 47(6): 330-336.
Hainsworth, J. D., S. Litchy, L. H. Morrissey, M. B. Andrews, M. Grimaldi, M. McCarty and F. A. Greco (2005). “Rituximab plus short-duration chemotherapy as first-line treatment for follicular non-Hodgkin's lymphoma: a phase II trial of the minnie pearl cancer research network.” J Clin Oncol 23(7): 1500-1506.
Hennessy, E. J., A. E. Parker and L. A. O'Neill (2010). “Targeting Toll-like receptors: emerging therapeutics?” Nat Rev Drug Discov 9(4): 293-307.
Hsu, F. J., C. B. Caspar, D. Czerwinski, L. W. Kwak, T. M. Liles, A. Syrengelas, B. Taidi-Laskowski and R. Levy (1997). “Tumor-specific idiotype vaccines in the treatment of patients with B-cell lymphoma—long-term results of a clinical trial.” Blood 89(9): 3129-3135.
Huston, J. S., D. Levinson, M. Mudgett-Hunter, M. S. Tai, J. Novotny, M. N. Margolies, R. J. Ridge, R. E. Bruccoleri, E. Haber, R. Crea and et al. (1988). “Protein engineering of antibody binding sites: recovery of specific activity in an anti-digoxin single-chain Fv analogue produced in Escherichia coli.” Proc Natl Acad Sci USA 85(16): 5879-5883.
Inoges, S., A. L. de Cerio, H. Villanueva, F. Pastor, E. Soria and M. Bendandi (2011). “Idiotype vaccines for lymphoma: Potential factors predicting the induction of immune responses.” World J Clin Oncol 2(6): 237-244.
Inoges, S., A. L. de Cerio, H. Villanueva. E. Soria, F. Pastor and M. Bendandi (2011). “Idiotype vaccines for lymphoma therapy.” Expert Rev Vaccines 10(6): 801-809.
Inoges, S., A. Lopez-Diaz de Cerio, N. Zabalegui, E. Soria, H. Villanueva, C. Panizo, A. Rodriguez-Caballero, L. Suarez, F. Pastor, M. Rodriguez-Calvillo, A. Orfao and M. Bendandi (2009). “Prolonged idiotypic vaccination against follicular lymphoma.” Leuk Lymphoma 50(1): 47-53.
Inoges, S., M. Rodriguez-Calvillo, N. Zabalegui, A. Lopez-Diaz de Cerio, H. Villanueva, E. Soria, L. Suarez, A. Rodriguez-Caballero. F. Pastor, R. Garcia-Munoz, C. Panizo, J. Perez-Calvo, I. Melero, E. Rocha, A. Orfao and M. Bendandi (2006). “Clinical benefit associated with idiotypic vaccination in patients with follicular lymphoma.” J Natl Cancer Inst 98(18): 1292-1301.
Kanter, G., J. Yang, A. Voloshin, S. Levy, J. R. Swartz and R. Levy (2007). “Cell-free production of scFv fusion proteins: an efficient approach for personalized lymphoma vaccines.” Blood 109(8): 3393-3399.
Kim, K. J., C. Kanellopoulos-Langevin, R. M. Merwin, D. H. Sachs and R. Asofsky (1979). “Establishment and characterization of BALB/c lymphoma lines with B cell properties.” J Immunol 122(2): 549-554.
Krieg, A. M. (2006). “Therapeutic potential of Toll-like receptor 9 activation.” Nat Rev Drug Discov 5(6): 471-484.
Krieg, A. M. (2008). “Toll-like receptor 9 (TLR9) agonists in the treatment of cancer.” Oncogene27(2): 161-167.
Kwak, L. W., M. J. Campbell, D. K. Czerwinski, S. Hart, R. A. Miller and R. Levy (1992). “Induction of immune responses in patients with B-cell lymphoma against the surface-immunoglobulin idiotype expressed by their tumors.” N Engl J Med 327(17): 1209-1215.
Kwak, L. W., H. A. Young, R W. Pennington and S. D. Weeks (1996). “Vaccination with syngeneic, lymphoma-derived immunoglobulin idiotype combined with granulocyte/macrophage colony-stimulating factor primes mice for a protective T-cell response.” Proc Natl Acad Sci USA 93(20): 10972-10977.
Levy, R. (2008). Results of a Phase III trial evaluating safety and efficacy of specific immunotherapy, recombinant idiotype conjugated to KLH with GM-CSF, compared to non-specific immunotherapy, KLH with GM-CSF, in patients with follicular non-Hodgkin's lymphoma. American Association of Cancer Research Annual Meeting, LB-204.
Lim, S. H., S. A. Beers, R. R. French, P. W. Johnson, M. J. Glennie and M. S. Cragg (2010). “Anti-CD20 monoclonal antibodies: historical and future perspectives.” Haematologica 95(1): 135-143.
Lim, S. H., A. T. Vaughan, M. Ashton-Key, E. L. Williams, S. V. Dixon. H. T. Chan, S. A. Beers. R. R. French. K. L. Cox, A. J. Davies, K. N. Potter, C. I. Mockridge, D. G. Oscier, P. W. Johnson, M. S. Cragg and M. J. Glennie (2011). “Fc gamma receptor IIb on target B cells promotes rituximab internalization and reduces clinical efficacy.” Blood 118(9): 2530-2540.
Maloney, D. G., M. S. Kaminski, D. Burowski, J. Haimovich and R. Levy (1985). “Monoclonal anti-idiotype antibodies against the murine B cell lymphoma 38C13: characterization and use as probes for the biology of the tumor in vivo and in vitro.” Hybridoma 4(3): 191-209.
McCormick, A. A., S. Reddy, S. J. Reinl, T. I. Cameron, D. K. Czerwinkski, F. Vojdani, K. M. Hanley, S. J. Garger, E. L. White, J. Novak, J. Barrett, R. B. Holtz, D. Tuse and R. Levy (2008). “Plant-produced idiotype vaccines for the treatment of non-Hodgkin's lymphoma: safety and immunogenicity in a phase I clinical study.” Proc Natl Acad Sci USA 105(29): 10131-10136.
Miller, R. A., D. G. Maloney, R. Warnke and R. Levy (1982). “Treatment of B-cell lymphoma with monoclonal anti-idiotype antibody.” N Engl J Med 306(9): 517-522.
Milner, K., K. Mason, T. Theriault, M. Mayo and D. W. Denney (2007). Development of quantitative methods to assess humoral immune response (IR) in follicular Non-Hodgkin's Lymphoma (fNHL) patients receiving idiotype-keyhole limpet hemocyanin (Id-KLH) active immunotherapy. American Association of Cancer Research Annual Meeting, Poster #1857.
Mizel, S. B. and J. T. Bates (2010). “Flagellin as an adjuvant: cellular mechanisms and potential.” J Immunol 185(10): 5677-5682.
Murata, M. (2008). “Activation of Toll-like receptor 2 by a novel preparation of cell wall skeleton from Mycobacterium bovis BCG Tokyo (SMP-105) sufficiently enhances immune responses against tumors.” Cancer Sci 99(7): 1435-1440.
Patel, K. G., P. P. Ng, S. Levy, R. Levy and J. R. Swartiz (2010). “Escherichia coli-based production of a tumor idiotype antibody fragment—tetanus toxin fragment C fusion protein vaccine for B cell lymphoma.” Protein Expr Purif 75(1): 15-20.
Patel, K. G. and J. R. Swartz (2011). “Surface functionalization of virus-like particles by direct conjugation using azide-alkyne click chemistry.” Bioconjug Chem 22(3): 376-387.
Pumpens, P. and E. Grens (2001). “HBV core particles as a carrier for B cell/T cell epitopes.” Intervirology 44(2-3): 98-114.
Schuster, S. J., S. S. Neelapu, B. L. Gause, F. M. Muggia, J. P. Gockerman, E. M. Sotomayor, J. N. Winter, C. R. Flowers, A. M. Stergiou and J. W. Kwak (2009). Idiotype vaccine therapy (BiovaxID) in follicular lymphoma in first complete remission: BV301 Phase III clinical trial results. American Society of Clinical Oncology.
Siano, M., E. Lerch, L. Negretti, E. Zucca, D. Rodriguez-Abreu, M. Oberson. L. Leoncini, O. Mora, C. Sessa, A. Gallino and M. Ghielmini (2008). “A phase I-II study to determine the maximum tolerated infusion rate of rituximab with special emphasis on monitoring the effect of rituximab on cardiac function.” Clin Cancer Res 14(23): 7935-7939.
Spina, M., U. Jaeger, J. A. Sparano, R. Talamini, C. Simonelli, M. Michieli, G. Rossi, E. Nigra, M. Berretta, C. Cattaneo, A. C. Rieger, E. Vaccher and U. Tirelli (2005). “Rituximab plus infusional cyclophosphamide, doxorubicin, and etoposide in HIV-associated non-Hodgkin lymphoma: pooled results from 3 phase 2 trials.” Blood 105(5): 1891-1897.
Strable, E., D. E. Prasuhn, Jr., A. K. Udit, S. Brown. A. J. Link, J. T. Ngo, G. Lander, J. Quispe, C. S. Potter, B. Carragher, D. A. Tirrell and M. G. Finn (2008). “Unnatural amino acid incorporation into virus-like particles.” Bioconjug Chem 19(4): 866-875.
Voloshin, A. M. and J. R. Swartz (2005). “Efficient and scalable method for scaling up cell free protein synthesis in batch mode.” Biotechnol Bioeng 91(4): 516-521.
Witzig, T. E., A. M. Vukov, T. M. Habennann, S. Geyer, P. J. Kurtin, W. R. Friedenberg, W. L. White, H. I. Chalchal, P. J. Flynn, T. R. Fitch and D. A. Welker (2005). “Rituximab therapy for patients with newly diagnosed, advanced-stage, follicular grade I non-Hodgkin's lymphoma: a phase II trial in the North Central Cancer Treatment Group.” J Clin Oncol 23(6): 1103-1108.
Yang, J., G. Kanter, A. Voloshin, R. Levy and J. R. Swartz (2004). “Expression of active murine granulocyte-macrophage colony-stimulating factor in an Escherichia coli cell-free system.” Biotechnol Pro 20(6): 1689-1696.
Yang, J., G. Kanter, A. Voloshin, N. Michel-Reydellet, H. Velkeen, R. Levy and J. R. Swartz (2005). “Rapid expression of vaccine proteins for B-cell lymphoma in a cell-free system.” Biotechnol Bioeng 89(5): 503-511.
Zhou, Z. and C. J. Fahrni (2004). “A fluorogenic probe for the copper(I)-catalyzed azide-alkyne ligation reaction: modulation of the fluorescence emission via 3(n,pi)-1(pi,pi) inversion.” J Am Chem Soc 126(29): 8862-8863.
Zimmerman, D. H., H. Steiner, 3rd, R. Carmabula, E. Talor and K. S. Rosenthal (2012). “LEAPS therapeutic vaccines as antigen specific suppressors of inflammation in infectious and autoimmune diseases.” J Vaccines Vaccin 3(5).
Zimmermann, S., A. Dalpke and K. Heeg (2008). “CpG oligonucleotides as adjuvant in therapeutic vaccines against parasitic infections.” Int J Med Microbiol 298(1-2): 39-44.
Zlotnick, A., N. Cheng, J. F. Conway, F. P. Booy, A. C. Steven, S. J. Stahl and P. T. Wingfield (1996). “Dimorphism of hepatitis B virus capsids is strongly influenced by the C-terminus of the capsid protein.” Biochemistry 35(23): 7412-7421.
Zuker, M. (2003). “Mfold web server for nucleic acid folding and hybridization prediction.” Nucleic Acids Res 31(13): 3406-3415.

1. A VLP free of a viral genome comprising two or more display polypeptides, nucleic acid molecules, polymers of the nucleic acid molecules, lipopolysaccharides, lipopeptides, peptidoglycans and/or small molecules or a portion thereof which are selected from any of:
a. a tumor associated antigen and an immunostimulatory oligonucleotide comprising an unmethylated cytosine;
b. a tumor associated antigen and flagellin;
c. a tumor associated antigen, flagellin and an immunostimulatory oligonucleotide comprising an unmethylated cytosine;
d. a tumor associated antigen and interleukin 15 (IL-15);
e. a tumor associated antigen, IL-15 and an immunostimulatory oligonucleotide comprising an unmethylated cytosine;
f. a tumor associated antigen, GM-CSF, flagellin, and an immunostimulatory oligonucleotide comprising an unmethylated cytosine;
g. a tumor associated antigen and poly (I:C);
h. a tumor associated antigen, poly (I:C) and an immunostimulatory oligonucleotide comprising an unmethylated cytosine;
i. a tumor associated antigen and one or more Toll-like receptor (TLR) agonists;
j. a tumor associated antigen, GM-CSF and IL-15;
k. a tumor associated antigen and (S)-[2,3-Bis(palmitoyloxy)-(2-RS)-propyl]-N-palmitoyl-(R)-Cys-(S)-Ser-(S)-Lys4-OH lipohexapeptide (Pam3CSK4);
l. a tumor associated antigen and a lipopolysaccharide (LPS);
m. a tumor associated antigen and 3-(2-methylpropyl)-3,5,8-triazatricyclo[7.4.0.0^{2,6}]trideca-1(9),2(6),4,7,10,12-hexaen-7-amine (1-(2-methylpropyl)-1H-imidazo[4,5-c]quinolin-4-amine or imiquimod);
n. a tumor associated antigen, poly (I:C) and imiquimod;
o. a tumor associated antigen, Pam3CSK4, flagellin and an immunostimulatory oligonucleotide comprising an unmethylated cytosine; and
p. a tumor associated antigen, Pam3CSK4, flagellin, GM-CSF and an immunostimulatory oligonucleotide comprising an unmethylated cytosine.

2. (canceled)

3. A VLP free of a viral genome comprising two or more display polypeptides, nucleic acid molecules, polymers of the nucleic acid, lipopolysaccharides, lipopeptides, peptidoglycans and/or small molecules or a portion thereof selected from any of:
a. An Id antigen and a CpG-X;
b. An Id antigen and flagellin;
c. An Id antigen, flagellin and a CpG-X;
d. An Id antigen and interleukin 15 (IL-15);
e. An Id antigen, IL-15 and a CpG-X;
f. An Id antigen and granulocyte-macrophage colony-stimulating factor (GM-CSF);
g. An Id antigen, GM-CSF and a CpG-X;
h. An Id antigen, GM-CSF, flagellin, and a CpG-X;
i. An Id antigen and poly (I:C);
j. An Id antigen, poly (I:C) and a CpG-X;
k. An Id antigen and a Toll-like receptor (TLR) agonist;
l. An Id antigen and an immunostimulant;
m. An Id antigen, GM-CSF and IL-15;
n. An Id antigen and (S)-[2,3-Bis(palmitoyloxy)-(2-RS)-propyl]-N-palmitoyl-(R)-Cys-(S)-Ser-(S)-Lys4-OH lipohexapeptide (Pam3CSK4);
o. An Id antigen and a lipopolysaccharide (LPS);
p. An Id antigen and 3-(2-methylpropyl)-3,5,8-triazatricyclo[7.4.0.0^{2,6}]trideca-1(9),2(6),4,7,10,12-hexaen-7-amine (1-(2-methylpropyl)-1H-imidazo[4,5-c]quinolin-4-amine or imiquimod);
q. An id antigen, poly (I:C) and imiquimod;
r. An Id antigen, Pam3CSK4, flagellin and a CpG-X; and
s. An Id antigen, Pam3CSK4, flagellin, GM-CSF and a CpG-X.

4.-11. (canceled)

12. The VLP free of a viral genome of claim 3, wherein the Id antigen is an immunoglobulin expressed by a B-cell malignancy or a T-cell receptor expressed by a T-cell malignancy.

13. (canceled)

14. The VLP free of a viral genome of claim 12, wherein the B-cell malignancy is non-Hodgkin lymphoma, Hodgkin lymphoma, Burkitt's lymphoma, acute lymphocytic leukemias, lymphoblastic lymphomas, chronic lymphocytic leukemia (CLL), mantle cell lymphoma (MCL), multiple myeloma (MM), small lymphocytic lymphoma (SLL), B-cell prolymphocytic leukemia, lymphoplasmocytic leukemia, splenic marginal zone lymphoma, marginal zone lymphoma (extra-nodal or nodal), plasma cell neoplasms (e.g., plasma cell myeloma, plasmacytoma, monoclonal immunoglobulin deposition diseases, heavy chain diseases), mixed cell type diffuse aggressive lymphomas of adults, large cell type diffuse aggressive lymphomas of adults, large cell immunoblastic diffuse aggressive lymphomas of adults, small non-cleaved cell diffuse aggressive lymphomas of adults, or follicular lymphoma.

15. The VLP free of a viral genome of claim 12, wherein the T-cell malignancy is chronic lymphocytic leukemia (CLL), large granular lymphocyte leukemia (T gamma lymphoproliferative disease, mycosis fungoides/Sezary syndrome, diffuse aggressive lymphomas of adults, peripheral T-cell lymphomas (mixed cell type and large cell, immunoblastic), adult T-cell leukemia/lymphoma, angiocentric lymphomas (lymphomatoid granulomatosis polymorphic reticulosis, acute lymphocytic leukemia, or lymphoblastic lymphoma.

16.-17. (canceled)

18. The VLP free of a viral genome of claim 1, wherein the tumor-associated antigen is selected from the group consisting of 17-1 A, 707-AP, AFP, Annexin II, ART-4, BAGE, BAGE-1, b-catenin, BCG, bcr/abl, Bcr/abl el4a2 fusion junction, bcr-abl (polypeptide from translation of b3a2 transcript), bcr-abl (polypeptide from translation of b2a2 transcript), bcr-abl p210 (polypeptide from translation of b2a2 transcript), bcr-abl p210 (polypeptide from translation of b3a2 transcript), bullous pemphigoid antigen-1, CA 19-9, CA125, CA215, CAG-3 cancer peptide, CAMEL tumor antigen, Cancer-testis antigen, Caspase-8, CCL3, CCL4, CD16, CD20, CD3, CD30, CD55, CD63, CDC27, CDK-4, CDR3, CEA, cluster 5, cluster-5A, cyclin-dependent kinase-4, Cyp-B, DAM-1 0, DAM-6, Dek-cain, E7, EGFR, EGFRvII 1, EGP40, ELF2 M, EpCAM, FucGM 1, G250, GA733, GAGE, GAGE-1-8, gastrin cancer associated antigen, GD12, GD3, globoH, glycophorin, GM1, GM2, GM3, GnTV, Gn-T-V, gp100, Her-2/neu, HERV-K-ME, high molecular weight-associated antigen, high molecular weight proteoglycan (IMPG), HPV-16 E6, HPV-16 E7, HPVE6, HSP70-2M, HST-2, hTERT, human chorionic gonadotropin (HCG), Human milk fat globule (HMFG), iCE, KIAA0205, KK-LC-1, KM-HN-1, L6, LAGE-1, LcOse4Cer, LDLR/FUT, Lewis A, Lewis v/b, M protein, MAGE-1, MVC, MAGE-A1-12, MAGE-C2, MAHGE-3, MART-1/Melan-A, MCIR, ME491, MUC1, MUC2, mucin, MUM-1, MUM-2, MUM-3, mutated p53, Myosin, MZ2-E, N9 neuraminidase, NA88, NA88-A, nasopharyngeal carcinoma antigen, NGA, NK1/c-3, Novel bcr/ablk fusion BCR exons 1, 13, 14 with ABL, exons 4, NY-ESO-1/LAGE-2, NY-ESO-1b, OC125, osteosarcoma associated antigen-1, P15, p190 mimor ber-abl (ela2), p53, Pm1/RARa, Polysialic acid, PRAME tumor antigen, PSA, PSM, RU1, RU2, SAGE, SART-1, SART-2, SART-3, Sialyl LeA, Sp17, SSX-2, SSX-4, surface immunoglobulin, TAG-1, TAG-2, TEL/AML1, TP1, TRAG-3, TRP-1 (gp75), TRP-2, TRP2-INT2, hTRT, tumor associated glycoprotein-72 (TAG-72), tyrosinase, u-PA, WT1, and XAGE-1b, or an immunostimulatory fragment thereof.

19. The VLP free of a viral genome of claim 1, wherein the one or more TLR agonist(s) or a TLR agonist is selected from the group consisting of a TLR 2, 3, 4, 5, 7, 8, or 9 agonist.

20. The VLP free of a viral genome of claim 1, wherein the VLP comprises virus coat polypeptides modified to comprise at least one first unnatural amino acid at a site of interest and wherein the two or more display polypeptides are modified to comprise at least one second unnatural amino acid, wherein the first unnatural amino acid is different from, and reactive with the second unnatural amino acid.

21.-24. (canceled)

25. The VLP free of a viral genome of claim 1, wherein the VLP comprises virus coat proteins from a virus selected from the group consisting of a bacteriophage, adenovirus, coxsackievirus, hepatitis A virus, poliovirus, Rhinovirus, Herpes simplex virus, Varicella-zoster virus, Epstein-Barr virus, Human cytomegalovirus, Human herpes virus, Hepatitis B virus, Hepatitis C virus, yellow fever virus, dengue virus, West Nile virus, HIV, Influenza virus, Measles virus, Mumps virus, Parainfluenza virus, Respiratory syncytial virus, Human metapneumovirus, Human papillomavirus, Rabies virus, Rubella virus, Human bocarivus or Parvovirus, and Norovirus.

26.-30. (canceled)

31. The VLP free of a viral genome of claim 3, wherein the Id antigen is associated with an autoimmune disorder.

32. The VLP free of a viral genome of claim 31, wherein the autoimmune disorder is selected from the group consisting of myasthenia gravis, primary biliary cirrhosis, dilated cardiomyoapthy, myocarditis, autoimmune polyendocrine syndrome type I (APS-1), cystic fibrosis vasculitides, acquired hypoparathyroidism, Goodpasture syndrome, autoimmune hepatitis, Crohn disease, coronary artery disease, pemphigus foliaceus, pemphigus vulgaris, Guillain-Barre syndrome, type 1 diabetes, stiff man syndrome, Rasmussen encephalitis, autoimmune gastritis, Addison disease, type 1 diabetes, insulin hypoglycemic syndrome (Hirata disease), tacanthosis, systemic lupus erythematosus (SLE)), pernicious anemia, treatment-resistant lyme arthritis, polyneuropathy, multiple sclerosis, demyelinating disease, rheumatic fever, atopic dermatitis, autoimmune hypothyroidism, vitilago, autoimmune thyroiditis, autoimmune Hashimoto thyroiditis, and celiac disease.

33.-34. (canceled)

35. The VLP free of a viral genome of claim 3, wherein the Id antigen is associated with a cancer or paraneoplastic autoimmune disorder selected from the group consisting of neuropathy, small lung cell cancer, hepatocellular carcinoma, liver cancer, paraneoplastic pemphigus, paraneoplastic stiff man syndrome, paraneoplastic encephalomyelitis, subacute autonomic neuropathy, SLE, cancer-associated retinopathy, paraneoplastic opsoclonus myoclonus ataxia, lower motor neuron syndrome, and Lambert-Eaton myasthenic syndrome.

36. The VLP free of a viral genome of claim 1, wherein the tumor associated antigen is an Id antigen and wherein the two or more display polypeptides, nucleic acid molecules, polymers of the nucleic acid, lipopolysaccharides, lipopeptides, peptidoglycans and/or small molecules are selected from the group consisting of:
a. An Id antigen and a CpG-X;
b. An Id antigen and flagellin;
c. An Id antigen, flagellin and a CpG-X;
d. An Id antigen and interleukin 15 (IL-15);
e. An Id antigen, IL-15 and a CpG-X;
f. An Id antigen, GM-CSF, flagellin, and a CpG-X;
g. An Id antigen and poly (I:C);
h. An Id antigen, poly (I:C) and a CpG-X;
i. An Id antigen and a Toll-like receptor (TLR) agonist;
j. An Id antigen, GM-CSF and IL-15;
k. An Id antigen and (S)-[2,3-Bis(palmitoyloxy)-(2-RS)-propyl]-N-palmitoyl-(R)-Cys-(S)-Ser-(S)-Lys4-OH lipohexapeptide (Pam3CSK4);
l. An Id antigen and a lipopolysaccharide (LPS);
m. An Id antigen and 3-(2-methylpropyl)-3,5,8-triazatricyclo[7.4.0.0^{2,6}]trideca-1(9),2(6),4,7,10,12-hexaen-7-amine (1-(2-methylpropyl)-1H-imidazo[4,5-c]quinolin-4-amine or imiquimod);
n. An Id antigen, poly (I:C) and imiquimod;
o. An Id antigen, Pam3CSK4, flagellin and a CpG-X; and
p. An Id antigen, Pam3CSK4, flagellin, GM-CSF and a CpG-X.

37. (canceled)

38. (canceled)

39. The VLP free of a viral genome of claim 1, wherein the one or more immunostimulants is selected from the group consisting of a bacterial protein, an interferon and a cytokine or a fragment or portion thereof.

40.-41. (canceled)

42. The VLP free of a viral genome of claim 39, wherein the cytokine induces an immune response predominantly of the Th2 type and is selected from the group consisting of IL-4, IL-5, IL-6 and IL-10.

43.-59. (canceled)

60. A method for producing a VLP free of a viral genome protein comprising culturing a host vector system which comprises the nucleic acid molecule encoding the VLP of claim 1 under suitable culture conditions so as to produce the VLP free of a viral genome in the host and recovering the VLP free of a viral genome so produced.

61. A VLP free of a viral genome produced by the method of claim 60.

62. A composition comprising the VLP free of a viral genome of claim 1, in an effective immunizing amount and a suitable carrier.

63.-68. (canceled)

69. A method of inhibiting tumor cells which comprises contacting the tumor cells with an effective amount of the VLP free of a viral genome of claim 1 thereby inhibiting the tumor cells.

70.-164. (canceled)

165. A method of treating a tumor in a subject, which comprises administering to said subject an effective amount of the VLP free of a viral genome of claim 1 thereby treating the subject.

166.-185. (canceled)


SEQUENCE LISTING (168 sequences)

SEQ ID NO: 1 - DNA, 447 bp, Hepatitis B virus
  CDS (1)..(447): Sequence encoding first 149 amino acids of Hepatitis B virus capsid protein (HepB core protein; GenBank Accession number AAA45486.1) with amino acid substitution mutations at codons 66 and 76 (M66S and L76M)
atggacattgatccttacaaagagttcggcgcgaccgtagaacttctttcgtttctgccgagcgattttttcccgtcagttcgtgatctgctggacactgctgccgcactgtaccgtgatgctctcgagagcccggaacactgcagcccgcatcataccgcgctgcgtcaggcaatcctgtgctggggtgatttatccacgctggccacctgggtgggtacgaacatggaagatccggcttcgcgcgatctggttgtctcttatgtcaacaccaatgtgggcttgaagttccgtcagttgctttggtttcacatcagctgcctgacctttggacgcgaaactgtgctggaatatctggtgagctttggtgtctggatccgcaccccacctgcttatcgcccgcccaacgcgccgattctttcaacgctcccggaaactactgtggtg

SEQ ID NO: 2 - protein, 149 aa, Hepatitis B virus
MDIDPYKEFGATVELLSFLPSDFFPSVRDLLDTAAALYRDALESPEHCSPHHTALRQAILCWGDLSTLATWVGTNMEDPASRDLVVSYVNTNVGLKFRQLLWFHISCLTFGRETVLEYLVSFGVWIRTPPAYRPPNAPILSTLPETTVV

SEQ ID NO: 3 - DNA, 1509 bp, Artificial Sequence
  Coding sequence of C-terminally FLAG epitope- tagged Salmonella enterica phase 1 flagellin protein with amber stop codon at thr-240 (GenBank accession no. AAR10630.1) for incorporation of a non-canonical amino acid; sequence is synthesized in lab
atggcacaagtaattaacacgaactccctgtcgttactgacgcaaaataacctgaataaatcgcagtcggcactgggcacggcaattgaacgtctctcatcgggacttcgtatcaatagcgcgaaagacgatgccgctggacaggcaattgctaaccgcttcactgcgaacatcaaaggcctgactcaggcttcgcgtaacgctaacgacggtatcagtattgcgcagactacagaaggcgcgctgaacgagatcaataacaatttgcagcgcgtgcgtgaattggctgtgcaatccgcaaacagtaccaatagccaaagcgacctggacagtatccaagcagaaatcacccagcgcttgaatgagattgatcgcgtgagcggccagacacagtttaacggtgtgaaagtcctggcccaagacaatacccttacgatccaggtcggtgccaacgacggagaaacgattgacattgatctcaagcagatcaacagccaaacgctgggcctggatacccttaacgtacagcaaaagtataaagtgtctgatacagcagcgactgtgacaggatacgccgacacgactattgccctggataactctacttttaaagctagtgcgacaggtctggggggcaccgatcagaaaatcgacggtgatctgaaatttgatgatacgaccggcaaatactatgccaaagtcacggtcaccggcggttagggcaaagatggctattatgaggttagcgttgacaaaacgaacggtgaagttacgctggcgggtggtgctaccagcccgttgacggggggcctcccggctaccgcaaccgaggacgtcaaaaatgtgcaggtggcgaatgcagatctcactgaggcgaaagctgcactgactgcggctggcgttaccggtaccgcatctgtggtgaaaatgagctataccgataacaatggcaaaaccattgatggcggcctggcggttaaagtaggcgacgattactactcagcgacccagaacaaagatggttctatctctattaacacgacgaagtatacggcagatgacggtactagtaaaaccgccctgaacaaactgggcggggctgacggtaagacggaagtcgtatctatcggtggcaaaacctatgccgcttccaaagcggaaggtcacaacttcaaagcacagccagatttggcagaggccgccgcgacgaccacggagaacccgctccagaaaatcgatgcggcccttgcccaggtagacacgttacgtagtgatctgggcgcagtccagaatcgttttaatagcgcgattacaaatttgggtaacacagtaaacaatctcaccagcgcccgtagccgtatcgaagactcggattatgcgacggaagtgtccaacatgtcccgtgctcagatcttgcagcaagcggggacctctgtgctcgcgcaagctaaccaggttccgcaaaacgtcctgagcttactgcgtgattacaaggatgatgacgataaa

SEQ ID NO: 4 - protein, 503 aa, Artificial Sequence
  misc_feature (240)..(240): The ′Xaa′ at location 240 stands for a non- canonical amino acid.
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGXGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLRDYKDDDDK

SEQ ID NO: 5 - DNA, 408 bp, Artificial Sequence
  Coding sequence of C-terminally FLAG epitope- tagged Homo sapiens granulocyte-macrophage colony-stimulating factor (GM-CSF) with an amber stop codon at codon 95 (corresponding to thr-111 of GenBank accession no. AAA98768.1); sequence is synthesized in lab
atggcaccggcacgaagtccaagtccgagcactcagccgtgggaacatgtcaatgcgatccaggaagcgcgtcgtctgttgaacttgtctcgtgataccgcggcggagatgaacgaaacggttgaagtcattagcgagatgtttgacttgcaggaacccacgtgcctgcagacacgcctggaactctacaagcagggcctgcgcggctcgctgaccaaattaaagggcccgctgaccatgatggcatcccattacaaacagcactgccctccgacgccggagtagagctgcgcgacccaaatcatcacgtttgagtcatttaaggaaaatctgaaggattttctgttggtgatcccttttgactgctgggaacccgtccaagaagactacaaggatgatgatgataaa

SEQ ID NO: 6 - protein, 136 aa, Artificial Sequence
  misc_feature (95)..(95): The ′Xaa′ at location 95 stands for a non- canonical amino acid.
MAPARSPSPSTQPWEHVNAIQEARRLLNLSRDTAAEMNETVEVISEMFDLQEPTCLQTRLELYKQGLRGSLTKLKGPLTMMASHYKQHCPPTPEXSCATQIITFESFKENLKDFLLVIPFDCWEPVQEDYKDDDDK

SEQ ID NO: 7 - DNA, 369 bp, Artificial Sequence
  Coding sequence of C-terminally FLAG epitope- tagged Homo sapiens interleukin-15 (IL-15) with an amber stop codon at codon 114 (corresponding to thr-135 of GenBank accession no. CAA71044.1); sequence is synthesized in lab
atgaattgggtaaatgtgattagtgatctcaaaaaaattgaagatttgatccagagtatgcatattgacgcaaccctgtataccgagtccgatgtgcatccatcatgcaaggtaacagccatgaaatgttttctgctggaattacaggtgattagcttagaaagcggtgacgcatctattcatgataccgtcgaaaatttaatcatcttagcgaacaattcactgagctctaacggtaatgtgacggaatccgggtgcaaagaatgcgaggaattggaagagaaaaatatcaaagaattccttcaatcatttgtacacatcgtgcagatgttcattaattagagcgactacaaggatgatgatgacaaa

SEQ ID NO: 8 - protein, 123 aa, Artificial Sequence
  misc_feature (114)..(114): The ′Xaa′ at location 114 stands for a non- canonical amino acid.
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINXSDYKDDDDK

SEQ ID NO: 9 - DNA, 378 bp, Homo sapiens
  misc_feature (1)..(378): Sequence encoding N2517-H Id antigen
gaggtgcagctggtggagtctgggggagccttggtccagcctggggggtccctgagactctcctgtgcagcctctggattcacctttagggacttttggatgagttgggtccgccaggctccaggaaaggggctggagtgggtggccaacataagggaagatggaaatgacaaatactatgtgggcgctgtgaggggccggttcaccgtctccagagacaacgccaggaactcattgtatctgcaaatgaacagcctgagagccgaggacacggctgtttattactgtgcgagagatctagggggatattgtagtagtaccaactgcgaaggctactttgactactggggccagggcaccctggtcaccgtctcctca

SEQ ID NO: 10 - protein, 126 aa, Homo sapiens
EVQLVESGGALVQPGGSLRLSCAASGFTFRDFWMSWVRQAPGKGLEWVANIREDGNDKYYVGAVRGRFTVSRDNARNSLYLQMNSLRAEDTAVYYCARDLGGYCSSTNCEGYFDYWGQGTLVTVSS

SEQ ID NO: 11 - DNA, 319 bp, Homo sapiens
  misc_feature (1)..(319): Sequence encoding N2517-K Id antigen
gacatccagatgacccagtctccttccaccctgtctgcatctgtaggagacagagtcactatcacttgccgggccagtcagagtattagtacttggttggcctggtatcagcagaaaccagggagagcccctaagttgttgatctataaggcatctactttagaaagtggggtcccatcaaggttcagcggcagtggatctgggacagaattcactctcaccatcagcggcctgcagcctgatgattttgcaacttattactgccaacaatatgctgattatcggacgttcggccaagggaccaaggtggaaatcaaac

SEQ ID NO: 12 - protein, 106 aa, Homo sapiens
DIQMTQSPSTLSASVGDRVTITCRASQSISTWLAWYQQKPGRAPKLLIYKASTLESGVPSRFSGSGSGTEFTLTISGLQPDDFATYYCQQYADYRTFGQGTKVEIK

SEQ ID NO: 13 - DNA, 393 bp, Homo sapiens
  misc_feature (1)..(393): Sequence encoding N3803-H Id antigen
gaggtacaactggtggagtcggggggaggtgtggttcggcctggggggtcccggagattgtcctgtgcagcctctggattcagctttgatgattacggcatgagttgggtccgccaagctccagggaaggggctggagtgggtctctggtatcaagtggaatggtggtagtagaggttatttagactctgtgaagggccgattcaccatctccagagacaacgccaagaacttcctgtatctgcaaatgaacagtctgagagtcgaggacacggccgtgtatcactgtgcgagagatcccaagaattatcactatgatagtagtggttattatgactattattacttccctatggacgtctggggccaagggaccacggtcaccgtctcctca

SEQ ID NO: 14 - protein, 131 aa, Homo sapiens
EVQLVESGGGVVRPGGSRRLSCAASGFSFDDYGMSWVRQAPGKGLEWVSGIKWNGGSRGYLDSVKGRFTISRDNAKNFLYLQMNSLRVEDTAVYHCARDPKNYHYDSSGYYDYYYFPMDVWGQGTTVTVSS

SEQ ID NO: 15 - DNA, 322 bp, Homo sapiens
  misc_feature (1)..(322): Sequence encoding N3803-K Id antigen
gacatccagatgacccagtctccatcctccctgtctgcctctgtaggagacagagtcaccatcacttgccgggcaagtcaggacattagaaatgatttaggctggtatcagcagaaaccagggaaagcccctaagcgcctgatctatgctgcattcagtttgcaaagtggggtcccatcaaggttcagcggcagtggatctgggacagaattcactctcacaatcagcagcctgcagcctgaagattttgcaacttattactgtctacagcataatagttacccttggacgttcggccaagggaccaaggtggaaatcaaac

SEQ ID NO: 16 - protein, 107 aa, Homo sapiens
DIQMTQSPSSLSASVGDRVTITCRASQDIRNDLGWYQQKPGKAPKRLIYAAFSLQSGVPSRFSGSGSGTEFTLTISSLQPEDFATYYCLQHNSYPWTFGQGTKVEIK

SEQ ID NO: 17 - DNA, 367 bp, Homo sapiens
  misc_feature (1)..(367): Sequence encoding N4229-H Id antigen
gaggtgcagctggtggagtctgggggaggcttggtacagcctggagggtccctgagactctcctgtgcagcctctggattcaccttcagtagttatgaaatgaactgggtccgccaggctccagggaaggggctggagtgggtttcatacattagtagtagtggtagtaccatatactacgcagactctgtgaagggccgattcaccatctccagagacaacgccaagaactcactgtatctgcaaatgaacagcctgagagccgaggacacggctgtttattactgtgcgagagatgtattactatggttcggggaaggtgcctttgactactggggccagggcaccctggtcaccgtctcctcaa

SEQ ID NO: 18 - protein, 122 aa, Homo sapiens
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCARDVLLWFGEGAFDYWGQGTLVTVSS

SEQ ID NO: 19 - DNA, 331 bp, Homo sapiens
  misc_feature (1)..(331): Sequence encoding N4229-K Id antigen
gacatccagatgacccagtctccatcctcactgtctgcatctgtgggagacagagtcaccatcacttgtcgggcgagtcaggtcattagcaatcatttagcctggtttcagcagaaaccaggggaagcccctaagtccctgatctatgatgcttccactttgcaaagtggggtcccatcaaagttcagtggcagtggatctgggacagatttcactctcaccatcagcagcctgcagcctgaagatcttggaacttattactgccaacagtatgataatctcaggccaggattcactttcggccctgggaccaaagtggatatcaaacgta

SEQ ID NO: 20 - protein, 110 aa, Homo sapiens
DIQMTQSPSSLSASVGDRVTITCRASQVISNHLAWFQQKPGEAPKSLIYDASTLQSGVPSKFSGSGSGTDFTLTISSLQPEDLGTYYCQQYDNLRPGFTFGPGTKVDIKR

SEQ ID NO: 21 - DNA, 354 bp, Homo sapiens
  misc_feature (1)..(354): Sequence encoding N3210-H Id antigen
caggtgcagctacaacagtggggcgcaggactgttgaagccttcggagaccctatccctcgcctgcgctgtctatggtgggtccttcagtgataactactggagttggatccgccagtcccccgggaagggcctggagtggattggagaaatcaatcatagtggaagtaccaactacaacccgtccctcaagagtcgagtcgtcatatcagtggacacgtccaagaaccagttctccctgagcctgaagtctatgacagccgcggacacggctatttattactgtgcgagagtccctaagtggctacaattactcgactactggggccagggaaccctggtcaccgtctcctca

SEQ ID NO: 22 - protein, 118 aa, Homo sapiens
QVQLQQWGAGLLKPSETLSLACAVYGGSFSDNYWSWIRQSPGKGLEWIGEINHSGSTNYNPSLKSRVVISVDTSKNQFSLSLKSMTAADTAIYYCARVPKWLQLLDYWGQGTLVTVSS

SEQ ID NO: 23 - DNA, 322 bp, Homo sapiens
  misc_feature (1)..(322): Sequence encoding N3210-K Id antigen
gacatccagatgacccagtctccatcctcactgtctgcatctgtcggaggcagagtcaccatcacttgtcgggcgagtcaggatattagcacctggttagcctggtatcaacaaaaaccagggaaagcccctaagtccctgatctatgctgcatccactttgcatagtggggtcccatcaaggttcagcggcagtggatctgggacatttttcactctcaccatcagcagcctgcaacctgaagattttgcaacttattactgccaacaatataatacttaccctcggacttttggccaggggaccaagctggagatcaaac

SEQ ID NO: 24 - protein, 107 aa, Homo sapiens
DIQMTQSPSSLSASVGGRVTITCRASQDISTWLAWYQQKPGKAPKSLIYAASTLHSGVPSRFSGSGSGTFFTLTISSLQPEDFATYYCQQYNTYPRTFGQGTKLEIK

SEQ ID NO: 25 - DNA, 388 bp, Homo sapiens
  misc_feature (1)..(388): Sequence encoding N4367-H Id antigen
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagacaggtccagtatttctatagcagcagctggtacaatcgggtagtctggttcgacccctggggccagggcaccctggtcaccgtctcctcaa

SEQ ID NO: 26 - protein, 129 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARQVQYFYSSSWYNRVVWFDPWGQGTLVTVSS

SEQ ID NO: 27 - DNA, 322 bp, Homo sapiens
  misc_feature (1)..(322): Sequence encoding N4367-K Id antigen
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattcgcggacgttcggccaagggaccaaggtggaaatcaaac

SEQ ID NO: 28 - protein, 107 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNSYSRTFGQGTKVEIK

SEQ ID NO: 29 - DNA, 365 bp, Homo sapiens
  misc_feature (1)..(365): Sequence encoding N4473-H Id antigen
caggtcaccttgagggagtctggtcctgcgctggtgatacccacacagaccctcacactgacctgcaccttctctggattctcactcagagctagtccaatgtgtgtgagctgggtccgtcagcccccagggaaggccctggagtggcttggactcattgattgggatgatgaaaagtactacagcgcatctctgaggaccagactcaccatctccaaggacacctccaaaaaccaggtggtccttacaatgaccaatttggaccctgtggacacagccacctattattgtgcacggatgcaatacctttgggggagttatcgtcatttcgactactggggccagggaaccctggtcaccgtctc

SEQ ID NO: 30 - protein, 121 aa, Homo sapiens
QVTLRESGPALVIPTQTLTLTCTFSGFSLRASPMCVSWVRQPPGKALEWLGLIDWDDEKYYSASLRTRLTISKDTSKNQVVLTMTNLDPVDTATYYCARMQYLWGSYRHFDYWGQGTLVTV

SEQ ID NO: 31 - DNA, 309 bp, Homo sapiens
  misc_feature (1)..(309): Sequence encoding N4473-L Id antigen
tcctatgtgctgactcagccaccctcggtgtcagtggccccaggacagacggccacacttacctgtgggggagacaccattggaggtaatattgtacactggtaccaacagaagccaggccaggcccctgtggtggtcgtctatgatgacaccgagcggccctcagggaccccagagcgattctctggctccaactctgggaacacggccaccctgaccatcagcagggtcgaagccggggatgaggccgactattactgtcaggtgtgggatggtgataatgatcatcatgtgacattcggcggaggg

SEQ ID NO: 32 - protein, 103 aa, Homo sapiens
SYVLTQPPSVSVAPGQTATLTCGGDTIGGNIVHWYQQKPGQAPVVVVYDDTERPSGTPERFSGSNSGNTATLTISRVEAGDEADYYCQVWDGDNDHHVTFGGG

SEQ ID NO: 33 - DNA, 360 bp, Homo sapiens
  misc_feature (1)..(360): Sequence encoding N2650-H Id antigen
caggtacagctgcagcagtcaggtccaggactggtgaagccctcgcagaccctctcactcacctgcgccatctccggggacagtgtctctagcaacagtgctgcttggaactggatcaggcagtccccatcgagaggccttgagtggctgggaaggacatactacaggtccaagtggtattatgattttgcagtatctgtgaaaagtcgaataaacatcaacccagacacatccaagaaccagttctccctgcacttgaactctgtgactcccgaggacacggctgtgtattactgtgcaagggggttttggagtggttacccctcctggggccagggaaccctggtcaccgtctcctca

SEQ ID NO: 34 - protein, 120 aa, Homo sapiens
QVQLQQSGPGLVKPSQTLSLTCAISGDSVSSNSAAWNWIRQSPSRGLEWLGRTYYRSKWYYDFAVSVKSRININPDTSKNQFSLHLNSVTPEDTAVYYCARGFWSGYPSWGQGTLVTVSS

SEQ ID NO: 35 - DNA, 306 bp, Homo sapiens
  misc_feature (1)..(306): Sequence encoding N2650-L Id antigen
cagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgaaaataataagcgaccctcagagattcctgaccgattctctggctccaagtctggcacgtcagccaccctgggcatcaccggactccagactggggacgaggccgattattactgcggaacatgggataccagtgctggggtttggggggtgttc

SEQ ID NO: 36 - protein, 102 aa, Homo sapiens
QSVLTQPPSVSAAPGQKVTISCSGSSSNIGNNYVSWYQQLPGTAPKLLIYENNKRPSEIPDRFSGSKSGTSATLGITGLQTGDEADYYCGTWDTSAGVWGVF

SEQ ID NO: 37 - DNA, 369 bp, Homo sapiens
  misc_feature (1)..(369): Sequence encoding N3965-H Id antigen
caggtgcaactggtggagtctgggggaggcgtggtccagcctggggggtccctgagactctcctgtgcagcctctggattcaccttcagtagctatgctatgcactgggtccgccaggctccaggcaaggggctggactgggtggcagttatatcatatgatggaagcaataaatactacgcagactccgtgaagggccgattcaccatctccagagaccattccaagaacacgctgtatttgcaaatgaacagcctgagagctgacgacacggctgtgtattactgtgcgagagatctgcagctcagatatacctatgggtcggcctttgactcctggggccagggaaccctggtcaccgtctcctca

SEQ ID NO: 38 - protein, 123 aa, Homo sapiens
QVQLVESGGGVVQPGGSLRLSCAASGFTFSSYAMHWVRQAPGKGLDWVAVISYDGSNKYYADSVKGRFTISRDHSKNTLYLQMNSLRADDTAVYYCARDLQLRYTYGSAFDSWGQGTLVTVSS

SEQ ID NO: 39 - DNA, 328 bp, Homo sapiens
  misc_feature (1)..(328): Sequence encoding N3965-L Id antigen
tcctatgtgctgactcagccaccctcagtgtcagtggccccaggaaagacggccaggattatctgtgggggaaacaacgttgggagtaaaagtgtgcactggtaccagcagaagccaggccaggcccctgttctggtcatctattatgatagcgaccgtccctcagggatccctgagcgattctctggctccaattctgggaacacggccaccctgaccatcagcagggtcgaagccggggatgaggccgactattactgtcaggtgtgggatagcagtagtggtcatcaggtgttcggcggagggaccaagctgaccgtcctaggta

SEQ ID NO: 40 - protein, 109 aa, Homo sapiens
SYVLTQPPSVSVAPGKTARIICGGNNVGSKSVHWYQQKPGQAPVLVIYYDSDRPSGIPERFSGSNSGNTATLTISRVEAGDEADYYCQVWDSSSGHQVFGGGTKLTVLG

SEQ ID NO: 41 - DNA, 369 bp, Homo sapiens
  misc_feature (1)..(369): Sequence encoding N3573-H Id antigen
gaggtgcaggtggtggcgtctgggggagacttggtccagcctggggggtccctgagactctcctgtacagcctctggattcaccttcactcattattggatgagttgggtccgccaggctccagggaaggggccggagtgggtggccaacataaagcaagatggaaaggagaaacactatgtggactctgtgaagggccgattcaccatctccagagacaacgccaagaactccatatatctgcaaatggacagcctgagagtcgaagacacggctgtatattattgtgtgagagacggcccgccccaaagtccaaccagcgggtggttcgacccctggggccagggaaccctggtcaccgtctcctca

SEQ ID NO: 42 - protein, 123 aa, Homo sapiens
EVQVVASGGDLVQPGGSLRLSCTASGFTFTHYWMSWVRQAPGKGPEWVANIKQDGKEKHYVDSVKGRFTISRDNAKNSIYLQMDSLRVEDTAVYYCVRDGPPQSPTSGWFDPWGQGTLVTVSS

SEQ ID NO: 43 - DNA, 334 bp, Homo sapiens
  misc_feature (1)..(334): Sequence encoding N3573-L Id antigen
cagtctgtgttgacgcagccgccctcagtgtctggggccccagggcagagggtcaccatctcctgcactgggaacatttccaacatcggggcaggttatgatgtacaatggtaccagcagcttccaggaacagcccccaaactcctcatctatggtgacaccaatcggccctcaggggtccctgaccgattctacggctccaggtctggcacctcagcctccctggccatcgctgggctccaggctgaggatgaggctgattattactgccagtcatatgacaacagcctgagtgttgtggtcttcggcggggggaccaagctgaccgtcctag

SEQ ID NO: 44 - protein, 111 aa, Homo sapiens
QSVLTQPPSVSGAPGQRVTISCTGNISNIGAGYDVQWYQQLPGTAPKLLIYGDTNRPSGVPDRFYGSRSGTSASLAIAGLQAEDEADYYCQSYDNSLSVVVFGGGTKLTVL

SEQ ID NO: 45 - DNA, 336 bp, Homo sapiens
  misc_feature (1)..(336): Sequence encoding N4128-H Id antigen
caggtgcagctacagcagtggggcgcaggactgttgaagccttcggagaccctgtccctcacctgcactgtctatggtgggtcattcagtagttactactggacttggatccgccagcccccagggaaggggctggaatggattggggaaatcaatcatagtggaagtaccaactacaacccgtccctcgagagtcgagtcaccatatcagtagacacgtccaaggaccagttttccctgaaactgaactctgtgaccgccgcggacacggctgtgtattactgtgcgagtcgtctctggggatctccccgttaccactggtacttcgatctctgg

SEQ ID NO: 46 - protein, 112 aa, Homo sapiens
QVQLQQWGAGLLKPSETLSLTCTVYGGSFSSYYWTWIRQPPGKGLEWIGEINHSGSTNYNPSLESRVTISVDTSKDQFSLKLNSVTAADTAVYYCASRLWGSPRYHWYFDLW

SEQ ID NO: 47 - DNA, 331 bp, Homo sapiens
  misc_feature (1)..(331): Sequence encoding N4128-L Id antigen
cagtctgtgttgacgcagccgccctcagtgtctggggccccagggcagagggtcaccatctcctgcactgggagcagctccaacatcggggcaggttatgatgtacactggtaccagcaccttccaggaaaagcccccaaactcctcatctatgctaacaacaatcggccctcaggggtccctgaccgattctctggttccaagtctggcacctcagcctccctggccatcactgggctccaggctgaagatgaggctgattattcctgccagtcctatgacaacagcctgagttgggtgttcggcggagggaccaagctgaccgtcctag

SEQ ID NO: 48 - protein, 110 aa, Homo sapiens
QSVLTQPPSVSGAPGQRVTISCTGSSSNIGAGYDVHWYQHLPGKAPKLLIYANNNRPSGVPDRFSGSKSGTSASLAITGLQAEDEADYSCQSYDNSLSWVFGGGTKLTVL

SEQ ID NO: 49 - DNA, 963 bp, Artificial Sequence
  Sequence encoding 38C13FABhcIM9s-X fusion protein with amber codon, Mus musculus Id 38C13 heavy chain Fab fragment, Escherichia coli colicin E9 immunity protein IM9, and FLAG epitope; lab synthesized
atggaagtgaaacttgttgaaagtggtggtggactggttcaaccgggcggcagcctgtcacttagctgcgcagcatccggcttcacctttacggattactacatgtcctgggttcgccagccaccgggtaaagctctcgaatggttggctctgattcgtaataaagccaacggctataccacggaatattcggcgtcagttaaaggacgcttcaccatcagccgtgacaattctcaatctatcctgtatctgcagatgaatgctctgcgcgccgaagactcagctacgtactactgcgcgcgtgatcctaactattatgacggttcctatgagggatactttgactactggggtcaaggcacgaccctgaccgtgagcagtgcgaagaccacaccgccaagcgtctatccactcgccccaggatcagccgcgcagaccaacagcatggtgactctgggctgtctggttaagggttactttccggaaccagtgacagttacctggaactctggatcgctgagctcaggcgtacacacatttccggctgtactgcagtccgatttatataccctgagcagttctgtgacggttccgagtagcccgcgtccgagcgagactgttacctgtaatgtggcacacccggcgagctcaactaaagttgataaaaaaattgtgcctcgtgacgaactgaaacactcgattagtgattataccgaagcggaatttttacagttagtgacgaccatttctaacgcggatacgtagagcgaggaagagctggttaaactggtgacgcattttgaagaaatgaccgaacatccgtctggatctgacctgatttattatccgaaagaaggtgatgatgatagcccgagtggtatcgttaatacagtgaaacagtggcgcgcggctaacggaaaaagcggctttaaacagggagactacaaggacgatgatgacaaa

SEQ ID NO: 50 - protein, 321 aa, Artificial Sequence
  misc_feature (255)..(255): The ′Xaa′ at location 255 stands for propargyloxyphenylalanine.
MEVKLVESGGGLVQPGGSLSLSCAASGFTFTDYYMSWVRQPPGKALEWLALIRNKANGYTTEYSASVKGRFTISRDNSQSILYLQMNALRAEDSATYYCARDPNYYDGSYEGYFDYWGQGTTLTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDELKHSISDYTEAEFLQLVTTISNADTXSEEELVKLVTHFEEMTEHPSGSDLIYYPKEGDDDSPSGIVNTVKQWRAANGKSGFKQGDYKDDDDK

SEQ ID NO: 51 - DNA, 657 bp, Artificial Sequence
  Sequence encoding 38C13FABlc fusion protein with Mus musculus Id 38C13 light chain Fab fragment, and polyhistidine epitope tag; lab synthesized
atggatatacaaatgacgcaatctccgagttcactgagcgcaagcctgggtggtaaagtcacaattacctgcaaagcgagccaagatatcaataagtatattgcctggtaccagcataaaccaggtaaaggaccgcgtctgctgattcactacacctcgaccctgcaaccgggtattccttctcgtttctccggctcaggtagcggccgtgattatagcttcagtattagtaatctggagcccgaagatattgcgacctattactgcttacagtatgataacctttatacgttcggtggtggcactaaactggaaatcaaacgcgccgacgcggcgccaaccgttagcatcttccctccctctagcgaacagctgacgagcgggggtgcgtccgttgtgtgcttcctcaataacttttatccgaaagatattaacgtaaaatggaaaatcgatggttctgagcgccagaatggcgtactgaactcatggacagatcaggattcaaaagatagcacttatagtatgtcctccaccctgacactgactaaggatgaatacgaacgtcataatagttacacctgtgaagccactcataagaccagcaccagccctattgttaagagttttaatcgtaacgaacaccaccaccaccaccac

SEQ ID NO: 52 - protein, 219 aa, Artificial Sequence
  Synthetic Construct
MDIQMTQSPSSLSASLGGKVTITCKASQDINKYIAWYQHKPGKGPRLLIHYTSTLQPGIPSRFSGSGSGRDYSFSISNLEPEDIATYYCLQYDNLYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEHHHHHH

SEQ ID NO: 53 - DNA, 1023 bp, Artificial Sequence
  Sequence encoding 38C13ScFV IM9s-X fusion protein with amber codon, Mus musculus Id 38C13 single-chain variable fragment (scFV), Escherichia coli colicin E9 immunity protein IM9, and FLAG epitope; lab synthesized
atggatatacaaatgacgcaatctccgtcgagcctgagcgcgagtttgggcggcaaagtcaccattacctgcaaggcgtcccaggatattaataaatacattgcctggtaccagcacaaacctgggaaaggcccgcgtctgctgattcattacacgtcaaccttacagccgggaattccctctcgtttttccggctcgggctcaggtcgtgattactcgttcagtatcagtaaccttgaaccggaagacattgcgacctactactgcctgcagtacgataacctgtatacctttggcggcggcaccaagttagaaatcaagggaggtggcggttctgggggcggcggctcgggtggcggcgggagcgaagtcaaactggtggaatcgggtggcggactggtgcaacctggaggtagcctgtctttaagttgtgccgcttccggctttacctttacggattattatatgtcgtgggtgcgtcaacctccaggaaaagccctggagtggctggcgcttattcgcaataaagcaaatgggtataccacagaatattccgcaagcgtgaaaggtcgttttacgatttcgcgtgataacagccaatccattctgtacttacagatgaatgccctgcgtgcggaggattccgccacgtactattgtgcccgcgatccgaattactatgacggtagttacgagggctattttgattactggggccagggtactactctgaccgtgagttccgaactcaaacacagtatttccgattacaccgaagcggagtttctgcaacttgttaccacgatttctaacgcagatacctagtcggaagaggagctggtcaaattggtgacccactttgaagagatgaccgaacacccgtctggttctgatcttatttattaccctaaggaaggggatgatgactctccttcggggattgtaaatacagttaagcagtggcgtgccgcaaatggtaaaagcggctttaagcaaggagactacaaggacgatgacgacaag

SEQ ID NO: 54 - protein, 341 aa, Artificial Sequence
  misc_feature (275)..(275): The ′Xaa′ at location 275 stands for propargyloxyphenylalanine.
MDIQMTQSPSSLSASLGGKVTITCKASQDINKYIAWYQHKPGKGPRLLIHYTSTLQPGIPSRFSGSGSGRDYSFSISNLEPEDIATYYCLQYDNLYTFGGGTKLEIKGGGGSGGGGSGGGGSEVKLVESGGGLVQPGGSLSLSCAASGFTFTDYYMSWVRQPPGKALEWLALIRNKANGYTTEYSASVKGRFTISRDNSQSILYLQMNALRAEDSATYYCARDPNYYDGSYEGYFDYWGQGTTLTVSSELKHSISDYTEAEFLQLVTTISNADTXSEEELVKLVTHFEEMTEHPSGSDLIYYPKEGDDDSPSGIVNTVKQWRAANGKSGFKQGDYKDDDDK

SEQ ID NO: 55 - DNA, 1440 bp, Artificial Sequence
  Sequence encoding 38C13ScFVIM9-GMCSF fusion protein with Mus musculus granulocyte macrophage colony- stimulating factor (GM-CSF), E. coli colicin E9 immunity protein IM9, Mus musculus Id 38C13 scFv, FLAG epitope, and linkers; lab synthesized
atggcaccaacccgatcaccgatcaccgtgacgcgtccgtggaaacatgtggaagccatcaaagaagcactgaaccttctggacgacatgcccgtaaccctcaatgaagaggttgaagtcgtgagtaacgaattcagcttcaaaaagctgacctgcgtccagacccgcctgaaaatctttgagcagggcctgcgtgggaatttcacaaagttgaaaggtgcactgaatatgactgcttcatattaccagacctattgcccgccaacccctgagaccgattgcgaaacgcaagtgaccacatatgcggattttattgactcgttgaagacgtttctcaccgacattccattcgaatgtaaaaagccggtgcaaaaaggagggggtggctctgaactgaaacattcgattagcgactacaccgaggcggaattcctgcagctggtcacgacgatttgcaacgctgacacgagcagcgaagaggaactggtaaagttagtcacccactttgaagaaatgacggaacatccaagcggatccgatctgatctactatccgaaagaaggtgacgatgatagccccagtggcattgtgaacaccgtcaagcaatggcgtgccgcaaatggcaaatcggggtttaaacagggcggtggcggtggatcagacatccaaatgacgcaatcaccatcttctttgagcgcgtctctgggcggcaaagtaaccattacctgtaaggcgtctcaagacattaataaatacattgcatggtatcagcacaaaccgggcaaaggaccacgtctgctcattcattatacgtccaccctgcagccaggcattcctagtcgtttctccggctctggttctggacgtgattactccttttcaatctctaacctggaaccagaagatatcgccacctactactgcctgcagtacgataatctttacacttttggggggggtaccaaactcgaaatcaaggggggtggcggttctggtggcggtggtagcggggggggggggagcgaagttaagctggtcgaaagcggcggtgggctggtccagccgggcggttctctgtcgctgagttgtgccgcatcgggcttcacgttcaccgattactacatgagctgggtgcgtcagccgccgggcaaagccctcgaatggctggcgctgattcgtaacaaagccaacggatatacgacggaatattctgcgagcgtcaaaggccgctttaccattagtcgtgacaatagccagagtattctgtacttacagatgaacgcactgcgcgcagaggattcagctacttattactgtgcacgcgatccgaattattacgacggctcctacgaaggctattttgattactggggccaaggaaccacgctgacggtcagcagtggaggtggcggctccgactacaaggatgatgatgacaaa

SEQ ID NO: 56 - protein, 480 aa, Artificial Sequence
  Synthetic Construct
MAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQKGGGGSELKHSISDYTEAEFLQLVTTICNADTSSEEELVKLVTHFEEMTEHPSGSDLIYYPKEGDDDSPSGIVNTVKQWRAANGKSGFKQGGGGGSDIQMTQSPSSLSASLGGKVTITCKASQDINKYIAWYQHKPGKGPRLLIHYTSTLQPGIPSRFSGSGSGRDYSFSISNLEPEDIATYYCLQYDNLYTFGGGTKLEIKGGGGSGGGGSGGGGSEVKLVESGGGLVQPGGSLSLSCAASGFTFTDYYMSWVRQPPGKALEWLALIRNKANGYTTEYSASVKGRFTISRDNSQSILYLQMNALRAEDSATYYCARDPNYYDGSYEGYFDYWGQGTTLTVSSGGGGSDYKDDDDK

SEQ ID NO: 57 - DNA, 399 bp, Artificial Sequence
  muGMCSF T92X
atggcaccaacccgatcaccgatcactgttacgcgtccgtggaaacatgtggaggcgatcaaggaggcattaaatttactcgacgacatgcccgtgacgctgaatgaggaggttgaagttgttagtaatgaattcagcttcaagaagctgacctgtgttcaaacacgtttaaaaatttttgaacaggggctccgcggaaactttacaaaactgaaaggtgctctgaatatgaccgcctcttattatcaaacgtattgtccgccgacgccggagtaggattgcgaaacccaggtgacaacctatgcagactttattgattcactgaaaactttcttgactgatattccgtttgaatgcaaaaaaccaggtcaaaaagactacaaggatgatgacgataaa

SEQ ID NO: 58 - protein, 133 aa, Artificial Sequence
  muGMCSF T92X
MAPTRSPITVTRPWKHVEAIKEALNLLDDMPVTLNEEVEVVSNEFSFKKLTCVQTRLKIFEQGLRGNFTKLKGALNMTASYYQTYCPPTPEXDCETQVTTYADFIDSLKTFLTDIPFECKKPGQKDYKDDDDK

SEQ ID NO: 59 - DNA, 369 bp, Artificial Sequence
  muIL15 T114X
atgaattggattgacgtgcgctatgatcttgagaaaatcgaaagcctgatccaatcaattcacatcgacaccacgctgtacactgattccgattttcacccgtcttgcaaagttactgccatgaactgttttctcctggaattacaggtgattctgcatgagtactctaatatgaccttgaacgaaactgtacgtaacgtcttatacctggcgaatagcaccttgtcgagtaacaagaacgtggcggagagtggttgcaaagaatgtgaggagcttgaagaaaaaacttttacagagtttttacagtcttttattcgcatcgtccaaatgttcattaactagtccgactacaaggatgatgacgataaa

SEQ ID NO: 60 - protein, 123 aa, Artificial Sequence
  muIL15 T114X
MNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINXSDYKDDDDK

SEQ ID NO: 61 - DNA, 738 bp, Artificial Sequence
  sfGFP
atgtcaaaaggggaagaactgtttactggagtggtgccaattttagttgaactggacggcgacgtcaacggtcacaaattcagcgtgcgtggtgaaggcgaaggggatgccactatcggtaaactgacactgaagtttatctgtacgaccggcaaacttccggtgccttggccgacactggtgaccacactgacctatggtgtacaatgctttagtcgctaccccgatcacatgaagcgtcatgatttctttaaaagtgccatgccggaaggttatgttcaagaacgtaccatctctttcaaagacgatggcaaatacaaaacccgcgccgtcgtcaaatttgaaggggataccctggtgaaccgcattgaacttaaaggtaccgattttaaagaggatggcaatattctgggtcataaacttgaatataatttcaatagtcataacgtatacattacggctgataaacagaaaaacggcatcaaagctaattttactgtgcgccacaatgtggaagatggttctgttcaactcgctgatcactaccaacagaacacccctatcggggatggcccggtgctcttaccggacaaccattatttgagcacgcagaccgtgttgtcgaaagacccgaacgaaaagcgtgaccacatggttttgcacgaatacgtcaacgccgctggaattacgtggtcgcatccgcagtttgaaaaagactacaaggatgatgacgataaa

SEQ ID NO: 62 - protein, 246 aa, Artificial Sequence
  sfGFP
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKRDHMVLHEYVNAAGITWSHPQFEKDYKDDDDK

SEQ ID NO: 63 - DNA, 738 bp, Artificial Sequence
  sfGFP-I39X
atgtcaaaaggggaagaactgtttactggagtggtgccaattttagttgaactggacggcgacgtcaacggtcacaaattcagcgtgcgtggtgaaggcgaaggggatgccacttagggtaaactgacactgaagtttatctgtacgaccggcaaacttccggtgccttggccgacactggtgaccacactgacctatggtgtacaatgctttagtcgctaccccgatcacatgaagcgtcatgatttctttaaaagtgccatgccggaaggttatgttcaagaacgtaccatctctttcaaagacgatggcaaatacaaaacccgcgccgtcgtcaaatttgaaggggataccctggtgaaccgcattgaacttaaaggtaccgattttaaagaggatggcaatattctgggtcataaacttgaatataatttcaatagtcataacgtatacattacggctgataaacagaaaaacggcatcaaagctaattttactgtgcgccacaatgtggaagatggttctgttcaactcgctgatcactaccaacagaacacccctatcggggatggcccggtgctcttaccggacaaccattatttgagcacgcagaccgtgttgtcgaaagacccgaacgaaaagcgtgaccacatggttttgcacgaatacgtcaacgccgctggaattacgtggtcgcatccgcagtttgaaaaagactacaaggatgatgacgataaa

SEQ ID NO: 64 - protein, 246 aa, Artificial Sequence
  sfGFP-I39X
MSKGEELFTGVVPILVELDGDVNGHKFSVRGEGEGDATXGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKRDHMVLHEYVNAAGITWSHPQFEKDYKDDDDK

SEQ ID NO: 65 - DNA, 1080 bp, Artificial Sequence
  IM9-S27X-38C13scFv
atggagaaaaaaatcggaggaggtggctctgaactgaaacatagcatctccgactataccgaagcggagtttttacagctggtgaccacgatttgcaacgccgatacctagtcggaagaagagctggtgaaattagtgacgcattttgaagagatgaccgagcatccgagcggttccgatctgatttactatccgaaagagggcgatgacgatagcccgagtgggattgttaacaccgttaaacagtggcgtgcggccaatggtaaaagcgggtttaaacaagggggtggcggtggatccgatattcaaatgacccagagcccgagctccctgagcgcgagcctggggggcaaagtgaccattacctgcaaagcgagccaggacatcaataagtatatcgcgtggtatcagcataaacccggcaaaggccctcgtctgctgatccattataccagcacccttcagccgggcatcccgagccgttttagcggcagcggaagcggacgtgattatagcttcagcattagcaatctggaaccggaagatattgcaacgtactattgcctgcaatatgataatctgtatacctttgggggtgggaccaagcttgagattaaagggggaggcggctccggtggcggtgggagcggcggaggtggcagcgaagtgaaactagttgaatccggagggggtcttgtccagccagggggctcactgtcgctgtcatgcgcagcgagcggctttaccttcaccgattattacatgagctgggtgcgtcaaccgcctggcaaggcgctggagtggctggcgctgattcgtaataaagcgaacggctacacaacggaatatagtgcgagcgtgaaaggccgtttcaccatttctcgtgataatagccagagcattctgtatcttcagatgaatgcgctgcgtgcggaagatagcgcgacctattattgtgcgcgtgatcccaactattatgatggcagttatgaaggctattttgattattggggccagggtaccaccctgaccgtgagcagcggtggcggtggcagctggagccacccgcagttcgaaaag

SEQ ID NO: 66 - protein, 360 aa, Artificial Sequence
  IM9-S27X-38C13scFv
MEKKIGGGGSELKHSISDYTEAEFLQLVTTICNADTXSEEELVKLVTHFEEMTEHPSGSDLIYYPKEGDDDSPSGIVNTVKQWRAANGKSGFKQGGGGGSDIQMTQSPSSLSASLGGKVTITCKASQDINKYIAWYQHKPGKGPRLLIHYTSTLQPGIPSRFSGSGSGRDYSFSISNLEPEDIATYYCLQYDNLYTFGGGTKLEIKGGGGSGGGGSGGGGSEVKLVESGGGLVQPGGSLSLSCAASGFTFTDYYMSWVRQPPGKALEWLALIRNKANGYTTEYSASVKGRFTISRDNSQSILYLQMNALRAEDSATYYCARDPNYYDGSYEGYFDYWGQGTTLTVSSGGGGSWSHPQFEK

SEQ ID NO: 67 - DNA, 22 bp, Artificial Sequence
  CpG-X
tgactgtgaacgttcgagatga

SEQ ID NO: 68 - DNA, 22 bp, Artificial Sequence
  CpG-X
tgactgtgaacgttcgagatga

SEQ ID NO: 69 - DNA, 22 bp, Artificial Sequence
  CpG-X
tgactgtgaacgttcgagatga

SEQ ID NO: 70 - DNA, 22 bp, Artificial Sequence
  CpG-X
tgactgtgaacgttcgagatga

SEQ ID NO: 71 - DNA, 22 bp, Artificial Sequence
  CpG-X
tgactgtgaacgttcgagatga

SEQ ID NO: 72 - DNA, 22 bp, Artificial Sequence
  CpG-X
tgactgtgaacgttcgagatga

SEQ ID NO: 73 - DNA, 22 bp, Artificial Sequence
  CpG-X
tgactgtgaacgttcgagatga

SEQ ID NO: 74 - DNA, 22 bp, Artificial Sequence
  CpG-X
tgactgtgaacgttcgagatga

SEQ ID NO: 75 - DNA, 22 bp, Artificial Sequence
  CpG-X
tgactgtgaacgttcgagatga

SEQ ID NO: 76 - DNA, 22 bp, Artificial Sequence
  CpG-X
tgactgtgaacgttcgagatga

SEQ ID NO: 77 - DNA, 22 bp, Artificial Sequence
  CpG-X
tgactgtgaacgttcgagatga

SEQ ID NO: 78 - DNA, 22 bp, Artificial Sequence
  CpG-X
tgactgtgaacgttcgagatga

SEQ ID NO: 79 - DNA, 22 bp, Artificial Sequence
  CpG-X
tgactgtgaacgttcgagatga

SEQ ID NO: 80 - DNA, 22 bp, Artificial Sequence
  CpG-X
tgactgtgaacgttcgagatga

SEQ ID NO: 81 - DNA, 22 bp, Artificial Sequence
  CpG-X
tgactgtgaacgttcgagatga

SEQ ID NO: 82 - DNA, 22 bp, Artificial Sequence
  CpG-X
tgactgtgaacgttcgagatga

SEQ ID NO: 83 - protein, 13 aa, Artificial Sequence
  Id antigen
QAPGQGLEWMGXI

SEQ ID NO: 84 - protein, 13 aa, Artificial Sequence
  Id antigen
QAPGQGLEWVGXI

SEQ ID NO: 85 - protein, 13 aa, Artificial Sequence
  Id antigen
QAPGQGLEWIGXI

SEQ ID NO: 86 - protein, 13 aa, Artificial Sequence
  Id antigen
QAPGKGLEWMGXI

SEQ ID NO: 87 - protein, 13 aa, Artificial Sequence
  Id antigen
QAPGKGLEWVGXI

SEQ ID NO: 88 - protein, 13 aa, Artificial Sequence
  Id antigen
QAPGKGLEWIGXI

SEQ ID NO: 89 - protein, 13 aa, Artificial Sequence
  Id antigen
QALGQGLEWMGXI

SEQ ID NO: 90 - protein, 13 aa, Artificial Sequence
  Id antigen
QALGQGLEWVGXI

SEQ ID NO: 91 - protein, 13 aa, Artificial Sequence
  Id antigen
QALGQGLEWIGXI

SEQ ID NO: 92 - protein, 13 aa, Artificial Sequence
  Id antigen
QALGKGLEWMGXI

SEQ ID NO: 93 - protein, 13 aa, Artificial Sequence
  Id antigen
QALGKGLEWVGXI

SEQ ID NO: 94 - protein, 13 aa, Artificial Sequence
  Id antigen
QALGKGLEWIGXI

SEQ ID NO: 95 - protein, 13 aa, Artificial Sequence
  Id antigen
QPPGQGLEWMGXI

SEQ ID NO: 96 - protein, 13 aa, Artificial Sequence
  Id antigen
QPPGQGLEWVGXI

SEQ ID NO: 97 - protein, 13 aa, Artificial Sequence
  Id antigen
QPPGQGLEWIGXI

SEQ ID NO: 98 - protein, 13 aa, Artificial Sequence
  Id antigen
QPPGKGLEWMGXI

SEQ ID NO: 99 - protein, 13 aa, Artificial Sequence
  Id antigen
QPPGKGLEWVGXI

SEQ ID NO: 100 - protein, 13 aa, Artificial Sequence
  Id antigen
QPPGKGLEWIGXI

SEQ ID NO: 101 - protein, 13 aa, Artificial Sequence
  Id antigen
QPLGQGLEWMGXI

SEQ ID NO: 102 - protein, 13 aa, Artificial Sequence
  Id antigen
QPLGQGLEWVGXI

SEQ ID NO: 103 - protein, 13 aa, Artificial Sequence
  Id antigen
QPLGQGLEWIGXI

SEQ ID NO: 104 - protein, 13 aa, Artificial Sequence
  Id antigen
QPLGKGLEWMGXI

SEQ ID NO: 105 - protein, 13 aa, Artificial Sequence
  Id antigen
QPLGKGLEWVGXI

SEQ ID NO: 106 - protein, 13 aa, Artificial Sequence
  Id antigen
QPLGKGLEWIGXI

SEQ ID NO: 107 - protein, 13 aa, Artificial Sequence
  Id antigen
QAPGQGLEWMAXI

SEQ ID NO: 108 - protein, 13 aa, Artificial Sequence
  Id antigen
QAPGQGLEWVAXI

SEQ ID NO: 109 - protein, 13 aa, Artificial Sequence
  Id antigen
QAPGQGLEWIAXI

SEQ ID NO: 110 - protein, 13 aa, Artificial Sequence
  Id antigen
QAPGKGLEWMAXI

SEQ ID NO: 111 - protein, 13 aa, Artificial Sequence
  Id antigen
QAPGKGLEWVAXI

SEQ ID NO: 112 - protein, 13 aa, Artificial Sequence
  Id antigen
QAPGKGLEWIAXI

SEQ ID NO: 113 - protein, 13 aa, Artificial Sequence
  Id antigen
QALGQGLEWMAXI

SEQ ID NO: 114 - protein, 13 aa, Artificial Sequence
  Id antigen
QALGQGLEWVAXI

SEQ ID NO: 115 - protein, 13 aa, Artificial Sequence
  Id antigen
QALGQGLEWIAXI

SEQ ID NO: 116 - protein, 13 aa, Artificial Sequence
  Id antigen
QALGKGLEWMAXI

SEQ ID NO: 117 - protein, 13 aa, Artificial Sequence
  Id antigen
QALGKGLEWVAXI

SEQ ID NO: 118 - protein, 13 aa, Artificial Sequence
  Id antigen
QALGKGLEWIAXI

SEQ ID NO: 119 - protein, 13 aa, Artificial Sequence
  Id antigen
QPPGQGLEWMAXI

SEQ ID NO: 120 - protein, 13 aa, Artificial Sequence
  Id antigen
QPPGQGLEWVAXI

SEQ ID NO: 121 - protein, 13 aa, Artificial Sequence
  Id antigen
QPPGQGLEWIAXI

SEQ ID NO: 122 - protein, 13 aa, Artificial Sequence
  Id antigen
QPPGKGLEWMAXI

SEQ ID NO: 123 - protein, 13 aa, Artificial Sequence
  Id antigen
QPPGKGLEWVAXI

SEQ ID NO: 124 - protein, 13 aa, Artificial Sequence
  Id antigen
QPPGKGLEWIAXI

SEQ ID NO: 125 - protein, 13 aa, Artificial Sequence
  Id antigen
QPLGQGLEWMAXI

SEQ ID NO: 126 - protein, 13 aa, Artificial Sequence
  Id antigen
QPLGQGLEWVAXI

SEQ ID NO: 127 - protein, 13 aa, Artificial Sequence
  Id antigen
QPLGQGLEWIAXI

SEQ ID NO: 128 - protein, 13 aa, Artificial Sequence
  Id antigen
QPLGKGLEWMAXI

SEQ ID NO: 129 - protein, 13 aa, Artificial Sequence
  Id antigen
QPLGKGLEWVAXI

SEQ ID NO: 130 - protein, 13 aa, Artificial Sequence
  Id antigen
QPLGKGLEWIAXI

SEQ ID NO: 131 - protein, 13 aa, Artificial Sequence
  Id antigen
QAPGQGLEWMSXI

SEQ ID NO: 132 - protein, 13 aa, Artificial Sequence
  Id antigen
QAPGQGLEWVSXI

SEQ ID NO: 133 - protein, 13 aa, Artificial Sequence
  Id antigen
QAPGQGLEWISXI

SEQ ID NO: 134 - protein, 13 aa, Artificial Sequence
  Id antigen
QAPGKGLEWMSXI

SEQ ID NO: 135 - protein, 13 aa, Artificial Sequence
  Id antigen
QAPGKGLEWVSXI

SEQ ID NO: 136 - protein, 13 aa, Artificial Sequence
  Id antigen
QAPGKGLEWISXI

SEQ ID NO: 137 - protein, 13 aa, Artificial Sequence
  Id antigen
QALGQGLEWMSXI

SEQ ID NO: 138 - protein, 13 aa, Artificial Sequence
  Id antigen
QALGQGLEWVSXI

SEQ ID NO: 139 - protein, 13 aa, Artificial Sequence
  Id antigen
QALGQGLEWISXI

SEQ ID NO: 140 - protein, 13 aa, Artificial Sequence
  Id antigen
QALGKGLEWMSXI

SEQ ID NO: 141 - protein, 13 aa, Artificial Sequence
  Id antigen
QALGKGLEWVSXI

SEQ ID NO: 142 - protein, 13 aa, Artificial Sequence
  Id antigen
QALGKGLEWISXI

SEQ ID NO: 143 - protein, 13 aa, Artificial Sequence
  Id antigen
QPPGQGLEWMSXI

SEQ ID NO: 144 - protein, 13 aa, Artificial Sequence
  Id antigen
QPPGQGLEWVSXI

SEQ ID NO: 145 - protein, 13 aa, Artificial Sequence
  Id antigen
QPPGQGLEWISXI

SEQ ID NO: 146 - protein, 13 aa, Artificial Sequence
  Id antigen
QPPGKGLEWMSXI

SEQ ID NO: 147 - protein, 13 aa, Artificial Sequence
  Id antigen
QPPGKGLEWVSXI

SEQ ID NO: 148 - protein, 13 aa, Artificial Sequence
  Id antigen
QPPGKGLEWISXI

SEQ ID NO: 149 - protein, 13 aa, Artificial Sequence
  Id antigen
QPLGQGLEWMSXI

SEQ ID NO: 150 - protein, 13 aa, Artificial Sequence
  Id antigen
QPLGQGLEWVSXI

SEQ ID NO: 151 - protein, 13 aa, Artificial Sequence
  Id antigen
QPLGQGLEWISXI

SEQ ID NO: 152 - protein, 13 aa, Artificial Sequence
  Id antigen
QPLGKGLEWMSXI

SEQ ID NO: 153 - protein, 13 aa, Artificial Sequence
  Id antigen
QPLGKGLEWVSXI

SEQ ID NO: 154 - protein, 13 aa, Artificial Sequence
  Id antigen
QPLGKGLEWISXI

SEQ ID NO: 155 - protein, 21 aa, Artificial Sequence
  Id antigen comprising variable heavy domain.
YYMHWVRQAPGQGLEWMGRIN

SEQ ID NO: 156 - protein, 21 aa, Artificial Sequence
  Id antigen comprising variable heavy domain.
YYMHWVRQAPGQGLEWMGWIN

SEQ ID NO: 157 - protein, 21 aa, Artificial Sequence
  Id antigen comprising variable heavy domain.
YAISWVRQAPGQGLEWMGGII

SEQ ID NO: 158 - protein, 21 aa, Artificial Sequence
  Id antigen comprising variable heavy domain.
YTISWVRQAPGQGLEWMGRII

SEQ ID NO: 159 - protein, 21 aa, Artificial Sequence
  Id antigen comprising variable heavy domain.
YAISWVRQAPGQGLEWMGRII

SEQ ID NO: 160 - protein, 21 aa, Artificial Sequence
  Id antigen comprising variable heavy domain.
YWMSWVRQAPGKGLEWVANIK

SEQ ID NO: 161 - protein, 21 aa, Artificial Sequence
  Id antigen comprising variable heavy domain.
YAMSWVRQAPGKGLEWVSAIS

SEQ ID NO: 162 - protein, 21 aa, Artificial Sequence
  Id antigen comprising variable heavy domain.
YAMSWVRQAPGKGLEWVSAIY

SEQ ID NO: 163 - protein, 21 aa, Artificial Sequence
  Id antigen comprising variable heavy domain.
YAMSWVRQAPGKGLEWVSVIY

SEQ ID NO: 164 - protein, 21 aa, Artificial Sequence
  Id antigen comprising variable heavy domain.
YAMHWVRQAPGKGLEWVAVIS

SEQ ID NO: 165 - protein, 21 aa, Artificial Sequence
  Id antigen comprising variable heavy domain.
YYWSWIRQPPGKGLEWIGEIN

SEQ ID NO: 166 - protein, 21 aa, Artificial Sequence
  Id antigen comprising variable heavy domain.
YYWCWIRQPLGKGLEWIGEIN

SEQ ID NO: 167 - protein, 21 aa, Artificial Sequence
  Id antigen comprising variable heavy domain.
YYWSWIRQPPGKGLEWIGYIY

SEQ ID NO: 168 - protein, 21 aa, Artificial Sequence
  Id antigen comprising variable heavy domain.
YYWSWIRQPPGKGLEWIGEII